SEQUENCE LISTING (750 sequences)

SEQ ID NO: 1 - protein, 40 aa, Enterococcus faecalis
WLPPAGLLGRCGRWFRPWLLWLQSGAQYKWLGNLFGLGPK

SEQ ID NO: 2 - protein, 5 aa, Artificial Sequence
  N-terminal motif of class terminal IIa
  VARIANT 3: Xaa = any amino acid
YGXGV

SEQ ID NO: 3 - protein, 135 aa, Artificial Sequence
  Hybrid bacteriocin Ent35-MccV
GKYYGNGVSCNKKGCSVDWGRAIGIIGNNSAANLATGGAAGWKSGGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 4 - protein, 46 aa, Lactobacillus acidophilus
MISSHQKTLTDKELALISGGKTHYPTNAWKSLWKGFWESLRYTDGF

SEQ ID NO: 5 - DNA, 141 bp, Lactobacillus acidophilus
atgatttcatctcatcaaaaaacgttaactgataaagaattagcattaatttctggggggaaaacgcactacccgactaatgcatggaaaagtctttggaaaggtttctgggaaagccttcgttatactgacggtttttag

SEQ ID NO: 6 - protein, 81 aa, Lactobacillus acidophilus
MISMISSHQKTLTDKELALISGGKTYYGTNGVHCTKKSLWGKVRLKNVIPGTLCRKQSLPIKQDLKILLGWATGAFGKTFH

SEQ ID NO: 7 - DNA, 246 bp, Lactobacillus acidophilus
atgatttcaatgatttcatctcatcaaaaaacgttaactgataaagaattagcattaatttctggggggaaaacgtactatggtactaatggtgtgcattgtactaaaaagagtctttggggtaaagtacgcttaaaaaacgtgattcctggaactctttgtcgtaagcaatcgttgccgatcaaacaggatttaaaaattttactgggctgggctacaggtgcttttggcaagacatttcattaa

SEQ ID NO: 8 - protein, 60 aa, Lactobacillus acidophilus
MDKKTKILFEVLYIICIIGPQFILFVTAKNNMYQLVGSFVGIVWFSYIFWYIFFKQHKKM

SEQ ID NO: 9 - DNA, 183 bp, Lactobacillus acidophilus
atggataagaaaacaaaaatattatttgaagtattatacatcatctgtataataggccctcaatttatattatttgtgactgcaaaaaacaatatgtatcagttggtgggttcgtttgttggaatagtatggttttcgtatattttttggtatatttttttcaaacaacataaaaaaatgtag

SEQ ID NO: 10 - protein, 65 aa, Lactobacillus gasseri
MALKTLEKHELRNVMGGNKWGNAVIGAATGATRGVSWCRGFGPWGMTACALGGAAIGGYLGYKSN

SEQ ID NO: 11 - DNA, 198 bp, Lactobacillus gasseri
atggctttaaaaacattagaaaaacatgaattaagaaatgtaatgggtggaaacaagtgggggaatgctgtaataggagctgctacgggagctactcgcggagtaagttggtgcagaggattcggaccatggggaatgactgcctgtgcgttaggaggtgctgcaattggaggatatctgggatataagagtaattaa

SEQ ID NO: 12 - protein, 51 aa, Staphylococcus aureus [sequence omitted]

SEQ ID NO: 13 - DNA, 156 bp, Staphylococcus aureus
atgagttggttaaattttttaaaatacatcgctaaatatggcaaaaaagcggtatctgctgcttggaagtacaaaggtaaagtattagaatggcttaatgttggtcctactcttgaatgggtatggcaaaaattaaagaaaattgctggattataa

SEQ ID NO: 14 - protein, 61 aa, Enterococcus avium
MTRSKKLNLREMKNVVGGTYYGNGVSCNKKGCSVDWGKAISIIGNNSAANLATGGAAGWKS

SEQ ID NO: 15 - DNA, 186 bp, Enterococcus avium
atgacaagatcaaaaaaattaaatttacgcgaaatgaagaatgttgttggtggtacctactatggaaatggtgtatcttgtaacaagaaaggctgttcagttgactggggcaaagccatcagtattataggaaataattccgcagcaaacttagcaactggtggtgctgctggttggaagtcataa

SEQ ID NO: 16 - protein, 67 aa, Enterococcus faecalis
MKKKLVICGIIGIGFTALGTNVEAATYYGNGLYCNKQKCWVDWNKASREIGKIIVNGWVQHGPWAPR

SEQ ID NO: 17 - DNA, 204 bp, Enterococcus faecalis
atgaaaaagaaattagttatttgtggcattattgggattggttttacagcattaggaacaaatgtagaagctgctacgtattacggaaatggtttatattgtaataagcaaaaatgttgggtagactggaataaagcttcaagggaaattggaaaaattattgttaatggttgggtacaacatggcccttgggctcctagatag

SEQ ID NO: 18 - protein, 51 aa, Lactococcus lactis
MKEQNSFNLLQEVTESELDLILGAKGGSGVIHTISHEVIYNSWNFVFTCCS

SEQ ID NO: 19 - DNA, 156 bp, Lactococcus lactis
atgaaagaacaaaactcttttaatcttcttcaagaagtgacagaaagtgaattggaccttattttaggtgcaaaaggcggcagtggagttattcatacaatttctcatgaagtaatatataatagctggaactttgtatttacttgctgctcttaa

SEQ ID NO: 20 - protein, 74 aa, Enterococcus faecium
MKKKVLKHCVILGILGTCLAGIGTGIKVDAATYYGNGLYCNKEKCWVDWNQAKGEIGKIIVNGWVNHGPWAPRR

SEQ ID NO: 21 - DNA, 225 bp, Enterococcus faecium
atgaaaaagaaagtattaaaacattgtgttattctaggaatattaggaacttgtctagctggcatcggtacaggaataaaagttgatgcagctacttactatggaaatggtctttattgtaacaaagaaaaatgttgggtagattggaatcaagctaaaggagaaattggaaaaattattgttaatggttgggttaatcatggtccatgggcacctagaaggtag

SEQ ID NO: 22 - protein, 50 aa, Clostridium botulinum
MQKPEIISADLGLCAVNEFVALAAIPGGAATFAVCQMPNLDEIVSNAAYV

SEQ ID NO: 23 - DNA, 153 bp, Clostridium botulinum
atgcaaaaaccagaaattattagtgctgatttagggctttgtgcagttaatgaatttgtagctcttgctgccattcctggtggtgctgctacatttgcagtatgccaaatgccaaacttggatgagattgttagtaatgcagcatatgtttaa

SEQ ID NO: 24 - protein, 58 aa, Streptococcus equinus
MMNATENQIFVETVSDQELEMLIGGADRGWIKTLTKDCPNVISSICAGTIITACKNCA

SEQ ID NO: 25 - DNA, 177 bp, Streptococcus equinus
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcttgtaaaaattgtgcttaa

SEQ ID NO: 26 - protein, 77 aa, Brochothrix campestris
MHKVKKLNNQELQQIVGGYSSKDCLKDIGKGIGAGTVAGAAGGGLAAGLGAIPGAFVGAHFGVIGGSAACIGGLLGN

SEQ ID NO: 27 - DNA, 234 bp, Brochothrix campestris
atgcacaaggtaaaaaaattaaacaatcaagagttacaacagatcgtgggaggttacagttcaaaagattgtctaaaagatattggtaaaggaattggtgctggtacagtagctggggcagccggcggtggcctagctgcaggattaggtgctatcccaggagcattcgttggagcacattttggagtaatcggcggatctgccgcatgcattggtggattattaggtaactag

SEQ ID NO: 28 - protein, 80 aa, Butyrivibrio fibrisolvens
MSKKQIMSNCISIALLIALIPNIYFIADKMGIQLAPAWYQDIVNWVSAGGTLTTGFAIIVGVTVPAWIAEAAAAFGIASA

SEQ ID NO: 29 - DNA, 243 bp, Butyrivibrio fibrisolvens
atgagtaaaaaacaaattatgagtaactgtatatcaattgcattattaatagcactaattcctaatatctattttattgcagataaaatgggaattcagttagcacctgcttggtatcaagatattgtgaattgggtatctgctggtggaacacttactactggttttgcgattattgtaggagttacagtaccggcatggatagcagaagcagctgcagcttttggtatagcttcagcatga

SEQ ID NO: 30 - protein, 48 aa, Butyrivibrio fibrisolvens
MNKELNALTNPIDEKELEQILGGGNGVIKTISHECHMNTWQFIFTCCS

SEQ ID NO: 31 - DNA, 147 bp, Butyrivibrio fibrisolvens
atgaacaaagaacttaatgcacttacaaatcctattgacgagaaggagcttgagcagatcctcggtggtggcaatggtgtcatcaagacaatcagccacgagtgccacatgaacacatggcagttcattttcacatgttgctcttaa

SEQ ID NO: 32 - protein, 66 aa, Carnobacterium maltaromaticum
MNSVKELNVKEMKQLHGGVNYGNGVSCSKTKCSVNWGQAFQERYTAGINSFVSGVASGAGSIGRRP

SEQ ID NO: 33 - DNA, 201 bp, Carnobacterium maltaromaticum
atgaatagcgtaaaagaattaaacgtgaaagaaatgaaacaattacacggtggagtaaattatggtaatggtgtttcttgcagtaaaacaaaatgttcagttaactggggacaagcctttcaagaaagatacacagctggaattaactcatttgtaagtggagtcgcttctggggcaggatccattggtaggagaccgtaa

SEQ ID NO: 34 - protein, 61 aa, Carnobacterium maltaromaticum
MKSVKELNKKEMQQINGGAISYGNGVYCNKEKCWVNKAENKQAITGIVIGGWASSLAGMGH

SEQ ID NO: 35 - DNA, 186 bp, Carnobacterium maltaromaticum
atgaaaagcgttaaagaactaaataaaaaagaaatgcaacaaattaatggtggagctatctcttatggcaatggtgtttattgtaacaaagagaaatgttgggtaaacaaggcagaaaacaaacaagctattactggaatagttatcggtggatgggcttctagtttagcaggaatgggacattaa

SEQ ID NO: 36 - protein, 71 aa, Carnobacterium maltaromaticum
MNNVKELSIKEMQQVTGGDQMSDGVNYGKGSSLSKGGAKCGLGIVGGLATIPSGPLGWLAGAAGVINSCMK

SEQ ID NO: 37 - DNA, 216 bp, Carnobacterium maltaromaticum
atgaataatgtaaaagagttaagtattaaagaaatgcaacaagttactggtggagaccaaatgtcagatggtgtaaattatggaaaaggctctagcttatcaaaaggtggtgccaaatgtggtttagggatcgtcggcggattagctactatcccttcaggtcctttaggctggttagccggagcagcaggtgtaattaatagctgtatgaaataa

SEQ ID NO: 38 - protein, 64 aa, Carnobacterium maltaromaticum
MLYELVAYGIAQGTAEKVVSLINAGLTVGSIISILGGVTVGLSGVFTAVKAAIAKQGIKKAIQL

SEQ ID NO: 39 - DNA, 195 bp, Carnobacterium maltaromaticum
atgttatatgaattagttgcatatggtatcgcacaaggtacagctgaaaaggttgtaagtctaattaacgcaggtttaacagtagggtctattatttcaattttgggtggggtcacagtcggtttatcaggtgtcttcacagcagttaaagcagcaattgctaaacaaggaataaaaaaagcaattcaattataa

SEQ ID NO: 40 - protein, 836 aa, Pectobacterium carotovorum subsp. carotovorum
MIKYRLYAPNDGDTMTVSGGGGWVSNDDRKGGNDRDNGKGGSAVDFSKNPEKQAIVNPYLAIAIPMPVYPLYGKLGFTINTTAIETELANVRAAINTKLATLSAVIGRSLPVVGRVFGVTAAGMWPSSTAPSSLDSIYNQAHQQALAQLAAQQGVLNKGYNVTAMPAGFVSSLPVSEIKSLPTAPASLLAQSVINTELSQRQLALTQPTTNAPVANIPVVKAEKTAMPGVYSAKIIAGEPAFQIKVDNTKPALAQNPPKVKDDIQVSSFLSSPVADTHHAFIDFGSDHEPVYVSLSKIVTAEEEKKQVEEAKRREQEWLLRHPITAAERKLTEIRQVISFAQQLKESSVATISEKTKTVAVYQEQVNTAAKNRDNFYNQNRGLLSAGITGGPGYPIYLALWQTMNNFHQAYFRANNALEQESHVLNLARSDLAKAEQLLAENNRLQVETERTLAEEKEIKRNRVNVSTFGTVQTQLSKLLSDFYAVTSLSQSVPSGALASFSYNPQGMIGSGKIVGKDVDVLFSIPVKDIPGYKSPINLDDLAKKNGSLDLPIRLAFSDENGERVLRAFKADSLRIPSSVRGVAGSYDKNTGIFSAEIDGVSSRLVLENPAFPPTGNVGNTGNTAPDYKALLNTGVDVKPVDKITVTVTPVADPVDIDDYIIWLPTASGSGVEPIYVVFNSNPYGGTEKGKYSKRYYNPDKAGGPILELDWKNVKIDHAGVDNVKLHTGRFKASVENKVMIERLENILNGQITATDTDKRFYTHELRELNRYRNLGIKDGEVPSSIQEESAVWNDTHTATLEDYKINEKEQPLYTDAALQAAYEQELKDALGGKHG

SEQ ID NO: 41 - DNA, 2511 bp, Pectobacterium carotovorum subsp. carotovorum
atgattaaataccgtttatatgctccaaatgatggagacaccatgacagtgagtggtggtggtggttgggtttcaaacgatgatcgcaaaggtggtaatgacagggacaatggcaaaggtggttctgccgttgattttagtaaaaatccagaaaagcaggctatcgttaatccctatttggcaatcgcgataccgatgccggtctaccctctttatggaaagctagggttcacaataaatacgacggcaattgagactgaactcgcaaatgtcagagcagcaattaacactaaacttgcaacactcagtgcagtgattggcagatcacttccggtcgttgggcgggtatttggtgttactgccgccggaatgtggccttctagtaccgctcccagtagtctcgattctatatacaatcaagcacatcagcaggctttagcccagttagctgctcaacagggagtattaaataaagggtataacgttacagcaatgcctgcaggtttcgtcagcagtttgcctgttagtgaaatcaaatcattgccaacagctcccgccagtttactggcacaaagtgtgattaataccgaactttcccagcgtcaactggctcttactcagcccacgacgaatgcaccagtcgcgaatattcccgtagttaaagcagagaaaacagcaatgccaggtgtgtattcagcgaaaattattgctggtgagcctgcattccaaatcaaggtcgataataccaaacctgctttggcacagaatccgccgaaagtaaaagatgatattcaggtatcttctttcctttcctcgccagtagctgatacgcaccatgcatttattgattttggcagcgatcatgaaccggtatacgtgtctctttcaaagatcgtgacagccgaggaggagaaaaaacaggttgaagaggccaagcgccgtgagcaggagtggttgttgcgtcatccaattacagctgcggagcgaaaattaactgaaatccgccaagtgatctcttttgctcaacagctaaaagaaagctctgtcgcaaccatttcagaaaaaactaaaactgttgcggtttaccaagaacaggtgaataccgctgcaaaaaatcgcgacaatttttataatcaaaatagaggtctgttaagtgcgggtataactgggggaccgggatatcctatttatcttgctttatggcaaacgatgaataactttcatcaggcttatttcagagcaaataatgcattggaacaagagagtcatgttctgaacctggctcgttctgatctggctaaggctgagcaattgcttgctgagaataatcgacttcaggttgaaacggagcgaacgcttgccgaagaaaaagagataaaacgcaacagggttaatgtatcaacatttggcacagtgcaaactcaacttagtaaattgctgtcagatttttatgctgttacatcactttcccaaagtgttccttcgggggcattagcctctttttcatataatccacaagggatgattggcagcggtaagattgttgggaaggatgtcgatgttttattttccatcccagtaaaagatattccgggatataaatctcctattaacttggacgatttagccaagaaaaatggaagtctggatcttcccattcgtctggcattttctgatgagaatggagaaagggttcttcgggcattcaaagcggatagtctgcgaatcccttcgagtgtcagaggtgtagcgggcagttatgacaaaaatacgggtatttttagtgcagaaattgatggtgtttcatctcgccttgtactggaaaacccagcgtttcctccgaccggaaatgtcggtaatacgggtaatactgcacctgactataaagcattactgaatactggtgttgatgttaaacctgttgataaaatcacagttacggtaacaccagttgctgatccagtggatattgatgactatataatctggttgccaactgcgtctggttctggcgtggaacccatttatgtcgtgtttaacagtaatccgtatggtgggacggaaaaaggaaaatatagcaaacgttattataatccagataaggcaggcggtccgatcttggagctggattggaaaaacgttaagattgaccatgcaggtgtggacaatgttaaattacacacagggcgtttcaaagcgtcggttgaaaacaaagtgatgattgaacgtttggaaaacatactgaatggtcaaatcacggccacggatactgacaagcgattctatacgcatgaattaagagagttaaaccgctacagaaatttaggcatcaaagacggtgaagtgcctagtagcattcaagaagaaagcgctgtttggaacgacacacacacagcgacgcttgaagactacaaaattaatgagaaagagcaaccgttgtacactgatgctgctttgcaggcagcctacgaacaggaactcaaagacgcattaggagggaaacatggctaa

SEQ ID NO: 42 - protein, 74 aa, Bacillus cereus
MENLQMLTEEELMEIEGGGWWNSWGKCVAGTIGGAGTGGLGGAAAGSAVPVIGTGIGGAIGGVSGGLTGAATFC

SEQ ID NO: 43 - DNA, 225 bp, Bacillus cereus
atggaaaacttacaaatgttaactgaagaagaattaatggaaattgaaggtggaggctggtggaatagctggggtaaatgtgttgctggaactatcggtggagctggaactggtggtttaggtggagctgctgcaggttcagctgttccggttattggtactggtattggtggcgctattggtggagttagcggtggccttacaggtgcagctactttttgctaa

SEQ ID NO: 44 - protein, 78 aa, Streptoverticillium griseoverticillatum
MTASILQQSVVDADFRAALLENPAAFGASAAALPTPVEAQDQASLDFWTKDIAATEAFACRQSCSFGPFTFVCDGNTK

SEQ ID NO: 45 - DNA, 237 bp, Streptoverticillium griseoverticillatum
atgaccgcttccattcttcagcagtccgtcgtggacgccgacttccgcgcggcgctgcttgagaaccccgccgccttcggcgcttccgccgcggccctgcccacgcccgtcgaggcccaggaccaggcgtcccttgacttctggaccaaggacatcgccgccacggaagccttcgcctgccgccagagctgcagcttcggcccgttcaccttcgtgtgcgacggcaacaccaagtaa

SEQ ID NO: 46 - protein, 76 aa, Geobacillus kaustophilus
MSLLALVAGTLGVSQSIATTVVSIVLTGSTLISIILGITAILSGGVDAILEIGWSAFVATVKKIVAERGKAAAIAW

SEQ ID NO: 47 - DNA, 231 bp, Geobacillus kaustophilus
atgagtttgctggcgcttgttgccgggacgctcggcgtgtcacagtcaatcgcgacgacggttgtttcgattgtgttgaccggctccactctcatttctattattcttgggatcaccgctattttgtcaggtggagtcgacgccattttggaaattgggtggtcagcttttgtcgcgacggtgaaaaaaatagtggcggaacgaggaaaagcggcagcgattgcatggtaa

SEQ ID NO: 48 - protein, 309 aa, Clostridium tyrobutyricum
MRKVFLRSIISTLVMCAFVSSSFSVNADESKPNDEKIINNIENVTTTKDIVKSNKNNIVYLDEGVMSIPLSGRKPIAIKDDNNKEDLTVTLPIKNTGDISKISSNGTILYKNNSSNSSNIALQPKNDGFKALININDKLANKEYEFTFNLPKNSKLISAATYLGKEYDTKEVFVVDKNNIITSIISPAWAKDANGHNVSTYYKIVSNNKLVQVVEFTENTAFPVVADPNWTKIGKCAGSIAWAIGSGLFGGAKLIKIKKYIAELGGLQKAAKLLVGATTWEEKLHAGGYALINLAAELTGVAGIQANCF

SEQ ID NO: 49 - DNA, 930 bp, Clostridium tyrobutyricum
ttgagaaaagtatttttaagatcaataatttcaacattagttatgtgtgcatttgtttcaagcagcttttcagtaaatgcggatgaaagcaaaccaaatgatgaaaaaataattaataacatagaaaacgttactactactaaagatattgtaaaaagtaataaaaataatattgtatatttagatgaaggtgtaatgagtattccattgtctgggagaaaacccattgctattaaagatgataataataaagaagatttaactgttacattacctattaagaatactggagatatatctaaaattagtagtaatggtactattctgtataaaaataatagtagtaattcatctaatatagctttacaacctaaaaatgatggatttaaggctttaataaatattaatgataagttagctaataaagaatatgaatttacatttaatttacccaaaaacagtaaattaattagtgctgccacatatttgggtaaagaatatgatacaaaagaagtatttgtagtagacaaaaataatataattacgagtattattagtccagcttgggctaaagatgcaaatggacataatgtttctacttattataagatagtatcgaataataaattagtacaagttgttgaattcacagaaaatactgcattcccggtggtagctgatcctaattggactaaaattgggaaatgcgctgggtcaatagcatgggctataggttctggcctttttggtggagcaaagctaattaaaataaaaaaatatatagcagagcttggaggacttcaaaaagcagctaaattattagttggtgcaaccacttgggaagaaaaattacacgcaggcggttatgcattaattaacttagctgctgagctaacaggtgtagcaggtatacaagcaaattgtttttaa

SEQ ID NO: 50 - protein, 62 aa, Bacillus coagulans
MKKIEKLTEKEMANIIGGKYYGNGVTCGKHSCSVDWGKATTCIINNGAMAWATGGHQGTHKC

SEQ ID NO: 51 - DNA, 189 bp, Bacillus coagulans
atgaaaaaaattgaaaaattaactgaaaaagaaatggccaatatcattggtggtaaatactacggtaatggggttacttgtggcaaacattcctgctctgttgactggggtaaggctaccacctgcataatcaataatggagctatggcatgggctactggtggacatcaaggtactcataaatgctag

SEQ ID NO: 52 - protein, 490 aa, Escherichia coli
MDKVTDNSPDVESTESTEGSFPTVGVDTGDTITATLATGTENVGGGGGAFGGASESSAAIHATAKWSTAQLKKHQAEQAARAAAAEAALAKAKSQRDALTQRLKDIVNDALRANAARSPSVTDLAHANNMAMQAEAERLRLAKAEQKAREEAEAAEKALREAERQRDEIARQQAETAHLLAMAEAAEAEKNRQDSLDEEHRAVEVAEKKLAEAKAELAKAESDVQSKQAIVSRVAGELENAQKSVDVKVTGFPGWRDVQKKLERQLQDKKNEYSSVTNALNSAVSIRDAKKTEVQNAEIKLKEAKDALEKSQVKDSVDTMVGFYQYITEQYGEKYSRIAQDLAEKAKGSKFNSVDEALAAFEKYKNVLDKKFSKVDRDDIFNALESITYDEWAKHLEKISRALKVTGYLSFGYDVWDGTLKGLKTGDWKPLFVTLEKSAVDFGVAKIVALMFSFIVGAPLGFWGIAIITGIVSSYIGDDELNKLNELLGI

SEQ ID NO: 53 - DNA, 1473 bp, Escherichia coli
atggataaagtcactgataattctccagatgtggagagcacagaatctactgaggggtcattcccaactgttggggttgatactggcgatacgattacagcgacgcttgcaactggaactgaaaatgttggtggaggcggtggagcatttggtggggccagtgaaagttctgctgcgatacatgcaaccgctaaatggtctaccgcgcagttgaaaaaacatcaggctgaacaggctgcccgtgctgctgcggctgaggcagcattggcaaaagcgaaatctcagcgtgatgccctgactcaacgtctcaaggatattgttaatgacgctttacgtgctaatgccgctcgtagtccatcagtaactgaccttgctcatgccaataatatggcaatgcaggcagaggctgagcgtttgcgccttgcgaaggcagagcaaaaagcccgtgaagaagctgaagcagcagaaaaagcgctccgggaagcagaacgccaacgtgatgagattgcccgccaacaggctgaaaccgcgcatttgttagcaatggcggaggcagcagaggctgagaaaaatcgacaggattctcttgatgaagagcatcgggctgtggaagtggcagagaagaagctggctgaggctaaagctgaactggcgaaggccgaaagcgatgtacagagtaagcaagcgattgtttccagagttgcaggggagcttgaaaacgctcaaaaaagtgttgatgtgaaggttaccggatttcctggatggcgtgatgttcagaaaaaactggagagacaattgcaggataagaagaatgaatattcgtcagtgacgaatgctcttaattctgctgttagcattagagatgctaaaaaaacagaagttcagaatgctgagataaaattaaaagaagctaaggatgctcttgagaagagtcaggtaaaagactctgttgatactatggttgggttttatcaatatataaccgaacaatatggggaaaaatattccagaatagctcaggatttagctgaaaaggcgaagggtagtaaatttaatagtgttgatgaagcacttgctgcatttgaaaagtataaaaatgtactggataagaaattcagtaaggttgatagggatgatatttttaatgctttagagtctattacttatgatgagtgggccaagcatctagaaaagatctctagggctcttaaggttactggatatttgtctttcgggtatgatgtatgggatggtaccctaaagggattaaaaacaggagactggaagcctttatttgtcactctggagaagagcgcggtagatttcggcgtggcaaaaattgtggcattaatgtttagttttattgttggtgcgcctcttggcttctggggaattgcaattatcacaggtattgtttcttcttacataggggatgatgagttgaacaagcttaatgaattactaggtatttaa

SEQ ID NO: 54 - protein, 522 aa, Escherichia coli
METAVAYYKDGVPYDDKGQVIITLLNGTPDGSGSGGGGGKGGSKSESSAAIHATAKWSTAQLKKTQAEQAARAKAAAEAQAKAKANRDALTQRLKDIVNEALRHNASRTPSATELAHANNAAMQAEDERLRLAKAEEKARKEAEAAEKAFQEAEQRRKEIEREKAETERQLKLAEAEEKRLAALSEEAKAVEIAQKKLSAAQSEVVKMDGEIKTLNSRLSSSIHARDAEMKTLAGKRNELAQASAKYKELDELVKKLSPRANDPLQNRPFFEATRRRVGAGKIREEKQKQVTASETRINRINADITQIQKAISQVSNNRNAGIARVHEAEENLKKAQNNLLNSQIKDAVDATVSFYQTLTEKYGEKYSKMAQELADKSKGKKIGNVNEALAAFEKYKDVLNKKFSKADRDAIFNALASVKYDDWAKHLDQFAKYLKITGHVSFGYDVVSDILKIKDTGDWKPLFLTLEKKAADAGVSYVVALLFSLLAGTTLGIWGIAIVTGILCSYIDKNKLNTINEVLGI

SEQ ID NO: 55 - DNA, 1569 bp, Escherichia coli
atggaaaccgcggtagcgtactataaagatggtgttccttatgatgataagggacaggtaattattactcttttgaatggtactcctgacgggagtggctctggcggcggaggtggaaaaggaggcagtaaaagtgaaagttctgcagctattcatgcaactgctaaatggtctactgctcaattaaagaaaacacaggcagagcaggctgcccgggcaaaagctgcagcggaagcacaggcgaaagcaaaggcaaacagggatgcgctgactcagcgcctgaaggatatcgtgaatgaggctcttcgtcacaatgcctcacgtacgccttcagcaacagagcttgctcatgctaataatgcagctatgcaggcggaagacgagcgtttgcgccttgcgaaagcagaagaaaaagcccgtaaagaagcggaagcagcagaaaaggcttttcaggaagcagaacaacgacgtaaagagattgaacgggagaaggctgaaacagaacgccagttgaaactggctgaagctgaagagaaacgactggctgcattgagtgaagaagctaaagctgttgagatcgcccaaaaaaaactttctgctgcacaatctgaagtggtgaaaatggatggagagattaagactctcaattctcgtttaagctccagtatccatgcccgtgatgcagaaatgaaaacgctcgctggaaaacgaaatgaactggctcaggcatccgctaaatataaagaactggatgagctggtcaaaaaactatcaccaagagccaatgatccgcttcagaaccgtcctttttttgaagcaaccagacgacgggttggggccggtaagattagagaagaaaaacaaaaacaggtaacagcatcagaaacacgtattaaccggataaatgctgatataactcagatccagaaggctatttctcaggtcagtaataatcgtaatgccggtatcgctcgtgttcatgaagctgaagaaaatttgaaaaaagcacagaataatctccttaattcacagattaaggatgctgttgatgcaacagttagcttttatcaaacgctgactgaaaaatatggtgaaaaatattcgaaaatggcacaggaacttgctgataagtctaaaggtaagaaaatcggcaatgtgaatgaagctctcgctgcttttgaaaaatacaaggatgttttaaataagaaattcagcaaagccgatcgtgatgctatttttaatgcgttggcatcggtgaagtatgatgactgggctaaacatttagatcagtttgccaagtacttgaagattacggggcatgtttcttttggatatgatgtggtatctgatatcctaaaaattaaggatacaggtgactggaagccactatttcttacattagagaagaaagctgcagatgcaggggtgagttatgttgttgctttactttttagcttgcttgctggaactacattaggtatttggggtattgctattgttacaggaattctatgctcctatattgataagaataaacttaatactataaatgaggtgttagggatttaa

SEQ ID NO: 56 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGI

SEQ ID NO: 57 - DNA, 1881 bp, Escherichia coli
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatgattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatccgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttaa

SEQ ID NO: 58 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLKTARNALTRAEQQLTQQKNTPDGKTIVSPEKFPGRSSTNHSIVVSGDPRFAGTIKITTSAVIDNRANLNYLLTHSGLDYKRNILNDRNPVVTEDVEGDKKIYNAEVAEWDKLRQRLLDARNKITSAESAINSARNNVSARTNEQKHANDALNALLKEKENIRSQLADINQKIAEEKRKRDEINMVKDAIKLTSDFYRTIYDEFGKQASELAKELASVSQGKQIKSVDDALNAFDKFRNNLNKKYNIQDRMAISKALEAINQVHMAENFKLFSKAFGFTGKVIERYDVAVELQKAVKTDNWRPFFVKLESLAAGRAASAVTAWAFSVMLGTPVGILGFAIIMAAVSALVNDKFIEQVNKLIGI

SEQ ID NO: 59 - DNA, 1881 bp, Escherichia coli
atgtctgaccctgtacgtattacaaatcccggtgcagaatcgctgggatatgattcagatggccatgaaattatggccgttgatatttatgtaaaccctccacgtgtcgatgtctttcatggtaccccgcctgcatggagttccttcgggaacaaaaccatctggggtggaaacgagtgggtcgatgattccccaacccgaagtgatatcgaaaaaagggacaaggaaatcacagcgtacaaaaacacgctcagcgcgcagcagaaagagaatgagaataagcgtactgaagctggaaaacgcctttctgcggcaattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgatgccctccggctgcatacagagagccggatgctgtttgctgatgctgattctcttcgtatatctccccgcgaggccaggtcgttaatcgaacaggctgaaaaacggcagaaggatgcgcagaacgcagacaagaaggccgctgatatgcttgctgaatacgagcgcagaaaaggtattctggacacgcggttgtcagagctggaaaaaaatggcggggcagcccttgccgttcttgatgcacaacaggcccgtctgctcgggcagcagacacggaatgacagggccatttcagaggcccggaataaactcagttcggtgacggaatcgcttaagacggcccgtaatgcattaaccagagctgaacaacagctgacgcaacagaaaaacacgcctgacggcaaaacgatagtttcccctgaaaaattcccggggcgttcatcaacaaatcattctattgttgtgagtggtgatccgaggtttgccggtacgataaaaatcacaaccagcgcggtcatcgataaccgtgcaaacctgaattatcttctgacccattccggtctggactataaacgcaatattctgaatgaccggaatccggtggtgacagaggatgtggaaggtgacaagaaaatttataatgctgaagttgctgaatgggataagttacggcaacgattgcttgatgccagaaataaaatcacctctgctgaatctgcgataaattcggcgagaaataacgtcagtgccagaacaaatgaacaaaagcatgcaaatgacgctcttaatgccctgttgaaggaaaaagagaatatccgtagccagcttgctgacatcaatcagaaaatagctgaagagaaaagaaaaagggatgaaataaatatggtaaaggatgccataaaactcacctctgatttctacagaacgatatatgatgagttcggtaaacaagcatccgaacttgctaaggagctggcttctgtatctcaagggaaacagattaagagtgtggatgatgcactgaacgcttttgataaattccgtaataatctgaacaagaaatataacatacaagatcgcatggccatttctaaagccctggaagctattaatcaggtccatatggcggagaattttaagctgttcagtaaggcatttggttttaccggaaaagttattgaacgttatgatgttgctgtggagttacaaaaggctgtaaaaacggacaactggcgtccattttttgtaaaacttgaatcactggcagcaggaagagctgcttcagcagttacagcatgggcgttttccgtcatgctgggaacccctgtaggtattctgggttttgcaattattatggcggctgtgagtgcgcttgttaatgataagtttattgagcaggtcaataaacttattggtatctga

SEQ ID NO: 60 - protein, 271 aa, Escherichia coli
METLTVHAPSPSTNLPSYGNGAFSLSAPHVPGAGPLLVQVVYSFFQSPNMCLQALTQLEDYIKKHGASNPLTLQIISTNIGYFCNADRNLVLHPGISVYDAYHFAKPAPSQYDYRSMNMKQMSGNVTTPIVALAHYLWGNGAERSVNIANIGLKISPMKINQIKDIIKSGVVGTFPVSTKFTHATGDYNVITGAYLGNITLKTEGTLTISANGSWTYNGVVRSYDDKYDFNASTHRGIIGESLTRLGAMFSGKEYQILLPGEIHIKESGKR

SEQ ID NO: 61 - DNA, 816 bp, Escherichia coli
atggaaaccttaactgttcatgcaccatcaccatcaactaacttaccaagttatggcaatggtgcattttctctttcagcaccacatgtgcctggtgctggccctcttttagtccaggttgtttatagttttttccagagtccaaacatgtgtcttcaggctttaactcaacttgaggattacatcaaaaaacatggggccagcaaccctctcacattgcagatcatatcgacaaatattggttacttctgtaacgccgaccgaaatctggttcttcaccctggaataagcgtttatgacgcttaccacttcgcaaaaccagcgccaagtcaatatgactatcgctcaatgaatatgaaacaaatgagcggtaatgtcactacaccaattgtggcgcttgctcactatttatggggtaatggcgctgaaaggagcgttaatatcgccaacattggtcttaaaatttcccctatgaaaattaatcagataaaagacattataaaatctggtgtagtaggcacattccctgtttctacaaagttcacacatgccactggtgattataatgttattaccggtgcatatcttggtaatatcacactgaaaacagaaggtactttaactatctctgccaatggctcctggacttacaatggcgttgttcgttcatatgatgataaatacgattttaacgccagcactcaccgtggcattatcggagagtcgctcacaaggctcggggcgatgttttctggtaaagagtaccagatactgcttcctggtgaaattcacattaaagaaagtggtaagcgataa

SEQ ID NO: 62 - protein, 387 aa, Escherichia coli
MGSNGADNAHNNAFGGGKNPGIGNTSGAGSNGSASSNRGNSNGWSWSNKPHKNDGFHSDGSYHITFHGDNNSKPKPGGNSGNRGNNGDGASAKVGEITITPDNSKPGRYISSNPEYSLLAKLIDAESIKGTEVYTFHTRKGQYVKVTVPDSNIDKMRVDYVNWKGPKYNNKLVKRFVSQFLLFRKEEKEKNEKEALLKASELVSGMGDKLGEYLGVKYKNVAKEVANDIKNFHGRNIRSYNEAMASLNKVLANPKMKVNKSDKDAIVNAWKQVNAKDMANKIGNLGKAFKVADLAIKVEKIREKSIEGYNTGNWGPLLLEVESWIIGGVVAGVAISLFGAVLSFLPISGLAVTALGVIGIMTISYLSSFIDANRVSNINNIISSVIR

SEQ ID NO: 63 - DNA, 51 bp, Escherichia coli
gcaaatcgagtttcgaatataaataacattatatctagtgttattcgatga

SEQ ID NO: 64 - protein, 103 aa, Escherichia coli
MRTLTLNELDSVSGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 65 - DNA, 312 bp, Escherichia coli
atgagaactctgactctaaatgaattagattctgtttctggtggtgcttcagggcgtgatattgcgatggctataggaacactatccggacaatttgttgcaggaggaattggagcagctgctgggggtgtggctggaggtgcaatatatgactatgcatccactcacaaacctaatcctgcaatgtctccatccggtttaggaggaacaattaagcaaaaacccgaagggataccttcagaagcatggaactatgctgcgggaagattgtgtaattggagtccaaataatcttagtgatgtttgtttataa

SEQ ID NO: 66 - protein, 58 aa, Enterococcus columbae
MMNATENQIFVETVSDQELEMLIGGAGRGWIKTLTKDCPNVISSICAGTIITACKNCA

SEQ ID NO: 67 - DNA, 177 bp, Enterococcus columbae
atgatgaatgctactgaaaaccaaatttttgttgagactgtgagtgaccaagaattagaaatgttaattggtggtgcaggtcgtggatggattaagactttaacaaaagattgtccaaatgtgatttcttcaatttgtgcaggtacaattattacagcttgtaaaaattgtgcttaa

SEQ ID NO: 68 - protein, 59 aa, Lactobacillus curvatus
MNNVKELSMTELQTITGGARSYGNGVYCNNKKCWVNRGEATQSIIGGMISGWASGLAGM

SEQ ID NO: 69 - DNA, 180 bp, Lactobacillus curvatus
atgaataatgtaaaagaattaagtatgacagaattacaaacaattaccggcggtgctagatcatatggcaacggtgtttactgtaataataaaaaatgttgggtaaatcggggtgaagcaacgcaaagtattattggtggtatgattagcggctgggctagtggtttagctggaatgtaa

SEQ ID NO: 70 - protein, 64 aa, Streptomyces sp.
MRSEMTLTSTNSAEALAAQDFANTVLSAAAPGFHADCETPAMATPATPTVAQFVIQGSTICLVC

SEQ ID NO: 71 - DNA, 195 bp, Streptomyces sp.
gtgcgatctgagatgactcttacgagcacgaattccgctgaggctctggcggcgcaggactttgcgaacaccgttctcagcgcggcggccccgggcttccacgcggactgcgagacgccggccatggccaccccggccacgccgaccgtcgcccagttcgtgatccagggcagcacgatctgcctggtctgctga

SEQ ID NO: 72 - protein, 65 aa, Bacillus halodurans
MVNSKDLRNPEFRKAQGLQFVDEVNEKELSSLAGSGDVHAQTTWPCATVGVSVALCPTTKCTSQC

SEQ ID NO: 73 - DNA, 198 bp, Bacillus halodurans
atggtaaattcaaaagatttgcgtaatcctgaattccgcaaagcccaaggtctacaattcgttgacgaggtgaacgagaaggaactttcgtctctagctggttcaggagatgtgcatgcacaaacaacttggccttgcgctacagttggtgtctccgtagccttgtgcccaactacaaagtgtacaagccagtgctaa

SEQ ID NO: 74 - protein, 66 aa, Carnobacterium divergens
MKNLKEGSYTAVNTDELKSINGGTKYYGNGVYCNSKKCWVDWGQASGCIGQTVVGGWLGGAIPGKC

SEQ ID NO: 75 - DNA, 201 bp, Carnobacterium divergens
atgaaaaacttaaaagaaggttcatacactgctgttaatactgatgaattaaaaagtatcaatggtggaacaaaatattatgggaatggcgtttattgcaattctaaaaaatgttgggtagattggggacaagcttcaggttgtatcggtcaaactgttgttggcggatggctaggcggagctataccaggtaaatgctaa

SEQ ID NO: 76 - protein, 63 aa, Carnobacterium divergens
MIKREKNRTISSLGYEEISNHKLQEIQGGKGILGKLGVVQAGVDFVSGVWAGIKQSAKDHPNA

SEQ ID NO: 77 - DNA, 192 bp, Carnobacterium divergens
atgattaaaagagaaaagaacagaacaatttcttcccttggttatgaagaaatttctaatcataaattgcaagaaatacaaggtggaaaaggaattcttggtaaactaggagtagtacaggcaggagtggattttgtatcaggagtgtgggctggaataaaacagtctgccaaagatcatcctaatgcgtaa

SEQ ID NO: 78 - protein, 75 aa, Carnobacterium divergens
MKKQILKGLVIVVCLSGATFFSTPQQASAAAPKITQKQKNCVNGQLGGMLAGALGGPGGVVLGGIGGAIAGGCFN

SEQ ID NO: 79 - DNA, 228 bp, Carnobacterium divergens
atgaaaaaacaaattttaaaagggttggttatagttgtttgtttatctggggcaacatttttctcaacaccacaacaagcttctgctgctgcaccgaaaattactcaaaaacaaaaaaattgtgttaatggacaattaggtggaatgcttgctggagctttgggtggacctggcggagttgtgttaggtggtataggtggtgcaatagcaggaggttgttttaattaa

SEQ ID NO: 80 - protein, 72 aa, Enterococcus durans
MQTIKELNTMELQEIIGGENDHRMPYELNRPNNLSKGGAKCAAGILGAGLGAVGGGPGGFISAGISAVLGCM

SEQ ID NO: 81 - DNA, 219 bp, Enterococcus durans
atgcaaacgatcaaagaattgaacacgatggaattacaagaaataattggaggtgaaaatgaccatcggatgccttacgaattgaaccgtccaaataatttatccaaaggtggggctaagtgtgctgctggaatacttggcgctggactaggcgcagtaggcggtggacctggcggatttattagtgccggaatcagtgctgttcttggttgtatgtaa

SEQ ID NO: 82 - protein, 72 aa, Enterococcus durans
MQTIKELNTMELQKIIGGENDHRMPYELNRPNNLSKGGAKCAAGILGAGLGAVGGGPGGFISAGISAVLGCM

SEQ ID NO: 83 - DNA, 219 bp, Enterococcus durans
atgcaaacgatcaaagaattgaacacgatggaattacaaaaaataattggaggtgaaaatgaccatcggatgccttacgaattgaaccgtccaaataatttatccaaaggtggagctaagtgcgctgccggaatacttggtgctggattaggcgcagtaggcggtggacctggcggatttattagtgccggaatcagtgctgttcttggttgtatgtaa

SEQ ID NO: 84 - protein, 220 aa, Streptococcus dysgalactiae subsp. equisimilis
MKKLKRLVISLVTSLLVISSTVPALVYANETNNFAETQKEITTNSEATLTNEDYTKLTSEVKTIYTNLIQYDQTKNKFYVDEDKTEQYYNYDDESIKGVYLMKDSLNDELNNNNSSNYSEIINQKISEIDYVLQGNDINNLIPSNTRVKRSADFSWIQRCLEEAWGYAISLVTLKGIINLFKAGKFEAAAAKLASATAGRIAGMAALFAFVATCGATTVS

SEQ ID NO: 85 - DNA, 663 bp, Streptococcus dysgalactiae subsp. equisimilis
atgaaaaaattaaaacgtcttgttatctctcttgttacttcattactagtaatttcaagtacagttccagcacttgtttacgctaatgaaacaaataactttgcagaaactcaaaaagaaattacaacaaattcagaagcaacattaaccaatgaagactacactaaattaacttccgaagtaaaaacaatttatacaaatctgattcaatacgaccaaacaaaaaacaaattttacgtcgatgaagacaaaactgaacaatattataactacgatgatgaaagtataaaaggggtttatctcatgaaagatagtttgaacgatgagttaaacaataataactcttcaaactattctgaaataattaatcaaaaaatctctgaaattgactatgtccttcaaggaaacgatataaataatttaattcctagcaataccagagtaaaaagatcagcagatttttcttggattcaaagatgtctagaagaagcatggggatatgctattagtctagttactctaaaaggaataatcaatctatttaaagcaggaaaatttgaagctgctgctgctaaattagcttctgctacagcaggtagaatcgctggaatggctgccttatttgctttcgtagcaacttgcggtgcgacaactgtatcataa

SEQ ID NO: 86 - protein, 57 aa, Enterococcus faecalis
MKQYKVLNEKEMKKPIGGESVFSKIGNAVGPAAYWILKGLGNMSDVNQADRINRKKH

SEQ ID NO: 87 - DNA, 174 bp, Enterococcus faecalis
atgaagcaatataaagtattgaatgaaaaagaaatgaaaaaacctattgggggagagtcggtttttagtaaaataggtaatgctgtaggtccagctgcttattggattttaaaaggattaggtaatatgagtgatgtaaaccaagctgatagaattaatagaaagaaacattaa

SEQ ID NO: 88 - protein, 44 aa, Enterococcus faecalis
MGAIAKLVAKFGWPIVKKYYKQIMQFIGEGWAINKIIDWIKKHI

SEQ ID NO: 89 - DNA, 135 bp, Enterococcus faecalis
atgggagcaatcgcaaaattagtagcaaagtttggatggccaattgttaaaaagtattacaaacaaattatgcaatttattggagaaggatgggcaattaacaaaattattgattggatcaaaaaacatatttaa

SEQ ID NO: 90 - protein, 43 aa, Enterococcus faecalis
MGAIAKLVAKFGWPFIKKFYKQIMQFIGQGWTIDQIEKWLKRH

SEQ ID NO: 91 - DNA, 132 bp, Enterococcus faecalis
atgggagcaatcgcaaaattagtagcaaagtttggatggccatttattaaaaaattctacaaacaaattatgcagtttatcggacaaggatggacaatagatcaaattgaaaaatggttaaaaagacattga

SEQ ID NO: 92 - protein, 74 aa, Enterococcus faecalis
MLNKKLLENGVVNAVTIDELDAQFGGMSKRDCNLMKACCAGQAVTYAIHSLLNRLGGDSSDPAGCNDIVRKYCK

SEQ ID NO: 93 - DNA, 225 bp, Enterococcus faecalis
atgttaaataaaaaattattagaaaatggtgtagtaaatgctgtaacaattgatgaacttgatgctcaatttggtggaatgagcaaacgtgattgtaacttgatgaaggcgtgttgtgctggacaagcagtaacatatgctattcatagtcttttaaatcgattaggtggagactctagtgatccagctggttgtaatgatattgtaagaaaatattgtaaataa

SEQ ID NO: 94 - protein, 65 aa, Enterococcus faecium
MKHLKILSIKETQLIYGGTTHSGKYYGNGVYCTKNKCTVDWAKATTCIAGMSIGGFLGGAIPGKC

SEQ ID NO: 95 - DNA, 195 bp, Enterococcus faecium
atgaaacatttaaaaattttgtctattaaagagacacaacttatctatgggggtaccactcatagtggaaaatattatggaaatggagtgtattgcactaaaaataaatgtacggtcgattgggccaaggcaactacttgtattgcaggaatgtctataggtggttttttaggtggagcaattccagggaagtgc

SEQ ID NO: 96 - protein, 105 aa, Enterococcus faecalis
MVKENKFSKIFILMALSFLGLALFSASLQFLPIAHMAKEFGIPAAVAGTVLNVVEAGGWVTTIVSILTAVGSGGLSLLAAAGRESIKAYLKKEIKKKGKRAVIAW

SEQ ID NO: 97 - DNA, 318 bp, Enterococcus faecalis
atggttaaagaaaataaattttctaagatttttattttaatggctttgagttttttggggttagccttgtttagtgcaagtcttcagtttttgcccattgcacatatggctaaagagttcggtataccagcagcagttgcaggaactgtgcttaatgtagttgaagctggtggatgggtcactactattgtatcaattcttactgctgtaggtagcggaggtctttctttactcgctgcagcaggaagagagtcaattaaagcataccttaagaaagaaattaagaaaaaaggaaaaagagcagttattgcttggtaa

SEQ ID NO: 98 - protein, 71 aa, Enterococcus faecium
MQNVKELSTKEMKQIIGGENDHRMPNELNRPNNLSKGGAKCGAAIAGGLFGIPKGPLAWAAGLANVYSKCN

SEQ ID NO: 99 - DNA, 216 bp, Enterococcus faecium
atgcaaaatgtaaaagaattaagtacgaaagagatgaaacaaattatcggtggagaaaatgatcacagaatgcctaatgagttaaatagacctaacaacttatctaaaggtggagcaaaatgtggtgctgcaattgctgggggattatttggaatcccaaaaggaccactagcatgggctgctgggttagcaaatgtatactctaaatgcaactaa

SEQ ID NO: 100 - protein, 58 aa, Enterococcus mundtii
MKKLTSKEMAQVVGGKYYGNGVSCNKKGCSVDWGKAIGIIGNNSAANLATGGAAGWKS

SEQ ID NO: 101 - DNA, 177 bp, Enterococcus mundtii
ttgaagaaattaacatcaaaagaaatggcacaagtagtaggtggaaaatactacggtaatggagtctcatgtaataaaaaagggtgcagtgttgattggggaaaagctattggcattattggaaataattctgctgcgaatttagctactggtggagcagctggttggaaaagttaa

SEQ ID NO: 102 - protein, 44 aa, Enterococcus faecalis
MLAKIKAMIKKFPNPYTLAAKLTTYEINWYKQQYGRYPWERPVA

SEQ ID NO: 103 - DNA, 135 bp, Enterococcus faecalis
atgttagcaaaaattaaagcgatgattaagaagtttccgaacccttatactttagcagctaagctaacgacttacgaaattaattggtataaacaacaatacggtcgttatccttgggagcgccctgtagcataa

SEQ ID NO: 104 - protein, 71 aa, Enterococcus faecium
MRKKLFSLALIGIFGLVVTNFGTKVDAATRSYGNGVYCNNSKCWVNWGEAKENIAGIVISGWASGLAGMGH

SEQ ID NO: 105 - DNA, 216 bp, Enterococcus faecium
atgagaaaaaaattatttagtttagctcttattggaatatttgggttagttgtgacaaattttggtacaaaagttgatgcagctacgcgttcatatggtaatggtgtttattgtaataatagtaaatgctgggttaactggggagaagctaaagagaatattgcaggaatcgttattagtggctgggcttctggtttggcaggtatgggacattaa

SEQ ID NO: 106 - protein, 34 aa, Enterococcus faecium
MNFLKNGIAKWMTGAELQAYKKKYGCLPWEKISC

SEQ ID NO: 107 - DNA, 105 bp, Enterococcus faecium
atgaattttcttaaaaatggtatcgcaaaatggatgaccggtgctgaattgcaagcgtataaaaagaaatatggatgcttgccatgggaaaaaatttcttgttaa

SEQ ID NO: 108 - protein, 76 aa, Enterococcus faecalis
MKKKLVKGLVICGMIGIGFTALGTNVEAATYYGNGVYCNKQKCWVDWSRARSEIIDRGVKAYVNGFTKVLGGIGGR

SEQ ID NO: 109 - DNA, 231 bp, Enterococcus faecalis
atgaaaaagaaattagttaaaggcttagttatttgtggcatgattgggattggttttacagcattaggaacaaatgtagaagccgccacgtattacggaaatggtgtctattgcaataagcaaaaatgttgggtagattggagtagagcacgttctgaaattatagacagaggcgtaaaagcatacgtcaatggatttacgaaagtgttaggtggtataggtggaagataa

SEQ ID NO: 110 - protein, 60 aa, Enterococcus faecalis
MKKEELVGMAKEDFLNVICENDNKLENSGAKCPWWNLSCHLGNDGKICTYSHECTAGCNA

SEQ ID NO: 111 - DNA, 183 bp, Enterococcus faecalis
atgaaaaaagaagaattagtaggaatggctaaggaagactttttaaatgttatttgtgaaaatgacaacaaactagaaaatagtggagcaaaatgtccttggtggaatctttcttgtcatttaggcaatgatggtaaaatttgcacttattcacatgaatgtaccgcaggttgtaatgcataa

SEQ ID NO: 112 - protein, 61 aa, Enterococcus faecalis
MTELNKRLQLKRDVSTENSLKKISNTDETHGGVTTSIPCTVMVSAAVCPTLVCSNKCGGRG

SEQ ID NO: 113 - DNA, 186 bp, Enterococcus faecalis
atgactgaacttaacaaaagattacaattaaaaagagatgtttcaacagaaaatagtttgaaaaaaatttctaatactgatgaaacacatgggggagttactacatcaattccatgtacagtaatggttagtgcggcagtatgtcctacccttgtttgctcgaataaatgtggcggtagaggctag

SEQ ID NO: 114 - protein, 58 aa, Enterococcus faecium
MQNVKEVSVKEMKQIIGGSNDSLWYGVGQFMGKQANCITNHPVKHMIIPGYCLSKILG

SEQ ID NO: 115 - DNA, 177 bp, Enterococcus faecium
atgcaaaatgtaaaagaagtttctgtaaaagagatgaaacaaattatcggtggttctaatgatagtctttggtatggtgtaggacaatttatgggtaaacaagcaaactgtataacaaaccatcctgttaaacacatgataattcctggatattgtttatcgaaaattttagggtaa

SEQ ID NO: 116 - protein, 55 aa, Enterococcus faecium
MKKYNELSKKELLQIQGGIAPIIVAGLGYLVKDAWDHSDQIISGFKKGWNGGRRK

SEQ ID NO: 117 - DNA, 168 bp, Enterococcus faecium
atgaaaaaatataatgagttatctaaaaaagaacttctacagattcaaggaggaatagcacctattatagttgctggccttggctatttagtaaaagatgcatgggatcactcagatcaaataatctcaggatttaaaaaaggttggaatggtggacgtagaaaataa

SEQ ID NO: 118 - protein, 343 aa, Enterococcus faecalis
MKNILLSILGVLSIVVSLAFSSYSVNAASNEWSWPLGKPYAGRYEEGQQFGNTAFNRGGTYFHDGFDFGSAIYGNGSVYAVHDGKILYAGWDPVGGGSLGAFIVLQAGNTNVIYQEFSRNVGDIKVSTGQTVKKGQLIGKFTSSHLHLGMTKKEWRSAHSSWNKDDGTWFNPIPILQGGSTPTPPNPGPKNFTTNVRYGLRVLGGSWLPEVTNFNNTNDGFAGYPNRQHDMLYIKVDKGQMKYRVHTAQSGWLPWVSKGDKSDTVNGAAGMPGQAIDGVQLNYITPKGEKLSQAYYRSQTTKRSGWLKVSADNGSIPGLDSYAGIFGEPLDRLQIGISQSNPF

SEQ ID NO: 119 - DNA, 1032 bp, Enterococcus faecalis
atgaaaaatattttactttctattctaggggtattatctatcgttgtttctttggcgttttcttcttattctgtcaacgcagcttctaatgagtggtcgtggccactgggcaaaccatatgcgggaagatatgaagaaggacaacaattcgggaacactgcatttaaccgaggaggtacttatttccatgatgggtttgactttggttctgctatttatggaaatggcagtgtgtatgctgtgcatgatggtaaaattttatatgctggttgggatcctgtaggtggaggctcattaggtgcatttattgtactacaagcgggaaacacaaatgtgatttatcaagaatttagccgaaatgttggagatattaaagttagcactggacaaactgttaaaaaaggacagctgataggaaagtttacttctagtcatttacatttaggaatgacaaaaaaagaatggcgttctgctcattcttcttggaataaagatgatggcacttggtttaacccaattcctatacttcaaggaggatctacgcctacgcctccaaatccaggaccaaaaaatttcacaacaaatgttcgttacggattgcgggtcctcggaggttcatggttaccagaagtaaccaactttaacaataccaatgatggtttcgcaggttaccctaatcgtcaacatgatatgctttatataaaggtagataaagggcaaatgaaatatcgtgttcacacggctcaaagtggatggttgccttgggtaagtaaaggggataagagcgatacagtaaatggagcggcaggtatgcctggacaagcaattgatggtgttcagctaaactatataactcctaagggagaaaaattatcacaggcttactatcgttcacaaactacgaaacgatcaggctggttaaaagtaagtgcagataatggttctattcctggactagacagttatgcaggaatctttggagaaccgttggatcgcttgcaaataggtatttcacagtcaaatccattttaa

SEQ ID NO: 120 - protein, 56 aa, Staphylococcus epidermidis
MENKKDLFDLEIKKDNMENNNELEAQSLGPAIKATRQVCPKATRFVTVSCKKSDCQ

SEQ ID NO: 121 - DNA, 171 bp, Staphylococcus epidermidis
atggaaaacaaaaaagatttatttgatttagaaatcaaaaaagataatatggaaaataataatgaattagaagctcaatctcttggtcctgcaattaaggcaactagacaggtatgtcctaaagcaacacgttttgttacagtttcttgtaaaaaaagtgattgtcaatag

SEQ ID NO: 122 - protein, 51 aa, Staphylococcus epidermidis
MAAFMKLIQFLATKGQKYVSLAWKHKGTILKWINAGQSFEWIYKQIKKLWA

SEQ ID NO: 123 - DNA, 156 bp, Staphylococcus epidermidis
atggcagcatttatgaagttaattcagttcttagcaactaaaggtcaaaagtatgtttcacttgcatggaaacataaaggtactattttaaaatggattaacgccggtcaaagttttgaatggatttataaacaaatcaaaaaattatgggcataa

SEQ ID NO: 124 - protein, 52 aa, Staphylococcus epidermidis
MEAVKEKNDLFNLDVKVNAKESNDSGAEPRIASKFICTPGCAKTGSFNSYCC

SEQ ID NO: 125 - DNA, 159 bp, Staphylococcus epidermidis
atggaagcagtaaaagaaaaaaatgatctttttaatcttgatgttaaagttaatgcaaaagaatctaacgattcaggagctgaaccaagaattgctagtaaatttatatgtactcctggatgtgcaaaaacaggtagttttaacagttattgttgttaa

SEQ ID NO: 126 - protein, 55 aa, Staphylococcus epidermidis
MNNSLFDLNLNKGVETQKSDLSPQSASVLKTSIKVSKKYCKGVTLTCGCNITGGK

SEQ ID NO: 127 - DNA, 168 bp, Staphylococcus epidermidis
atgaataactcattattcgatttaaacctaaacaaaggtgtagaaactcaaaagagtgatttaagtccgcaatctgctagtgtcttgaagacttctattaaagtatctaaaaaatattgtaaaggtgttactttaacatgcggttgcaatattactggtggtaaataa

SEQ ID NO: 128 - protein, 52 aa, Staphylococcus gallinarum
MEAVKEKNELFDLDVKVNAKESNDSGAEPRIASKFLCTPGCAKTGSFNSYCC

SEQ ID NO: 129 - DNA, 159 bp, Staphylococcus gallinarum
atggaagcagtaaaagagaaaaatgaactttttgatcttgacgttaaagtaaatgcaaaagagtctaatgattcaggcgcagaaccacgaattgctagtaaatttttatgtactcctggatgtgccaaaacaggtagcttcaatagctactgttgttaa

SEQ ID NO: 130 - protein, 63 aa, Lactococcus garvieae
MENNNYTVLSDEELQKIDGGIGGALGNALNGLGTWANMMNGGGFVNQWQVYANKGKINQYRPY

SEQ ID NO: 131 - DNA, 192 bp, Lactococcus garvieae
atggaaaacaacaattacacagtactttcagatgaagaactacaaaaaattgatggtggaatcggcggggctcttggtaatgctctcaacggattaggtacctgggcaaacatgatgaacggtggaggatttgttaatcagtggcaagtttatgctaataaaggaaaaataaatcaataccgtccgtattaa

SEQ ID NO: 132 - protein, 63 aa, Lactococcus garvieae
MFDLVATGMAAGVAKTIVNAVSAGMDIATALSLFSGAFTAAGGIMALIKKYAQKKLWKQLIAA

SEQ ID NO: 133 - DNA, 192 bp, Lactococcus garvieae
atgtttgatttagtcgcgactggaatggctgcaggtgtagcaaaaactattgttaatgccgttagtgctggtatggatattgccactgctttatcattgttctcaggagcttttactgcagctgggggaattatggcactcattaaaaaatatgctcaaaagaaattatggaaacagcttattgctgcataa

SEQ ID NO: 134 - protein, 91 aa, Lactobacillus gasseri
MVTKYGRNLGLNKVELFAIWAVLVVALLLTTANIYWIADQFGIHLATGTARKLLDAMASGASLGTAFAAILGVTLPAWALAAAGALGATAA

SEQ ID NO: 135 - DNA, 276 bp, Lactobacillus gasseri
atggttactaagtacggacgtaatttaggtttgaacaaggtagagttgtttgcaatttgggcggttttagtagttgctcttttattgaccacagcgaacatttattggattgctgatcaattcgggattcatttagcgactggaacagcccgtaagttattagatgcaatggcttctggtgcctcattgggaactgcctttgctgctattttgggcgtgacattacctgcatgggctttggcagctgcaggagcattgggagcgactgcagcctag

SEQ ID NO: 136 - protein, 75 aa, Lactobacillus gasseri
MKNFNTLSFETLANIVGGRNNWAANIGGVGGATVAGWALGNAVCGPACGFVGAHYVPIAWAGVTAATGGFGKIRK

SEQ ID NO: 137 - DNA, 228 bp, Lactobacillus gasseri
atgaaaaattttaatacattatcatttgaaacattggctaacatagttggtgggagaaataattgggctgctaatataggtggagtaggtggagcgacagtcgctggatgggctcttggaaatgcagtttgcggtcctgcttgtggctttgttggagcacactatgttccaatagcatgggctggcgtaacggcagctactggtggattcggaaagataagaaagtag

SEQ ID NO: 138 - protein, 64 aa, Lactobacillus plantarum
MSKLVKTLTISEISKAQNNGGKPAWCWYTLAMCGAGYDSGTCDYMYSHCFGIKHHSSGSSSYHC

SEQ ID NO: 139 - DNA, 195 bp, Lactobacillus plantarum
atgagtaaattggttaagacacttactataagtgaaatttctaaggctcaaaacaacggtggaaaacctgcatggtgttggtatactttagcaatgtgtggtgctggttatgattcgggaacctgtgattatatgtattcgcattgttttggtataaagcatcatagtagtggtagtagcagttatcattgttag

SEQ ID NO: 140 - protein, 359 aa, Haloferax mediterranei
MSKDRDGRRTSRRGTLKKIGGFSLGALSFGAVGRTQAATGSSVTTADIAPPGPNGDPKSVQIDDKYTGAEMYGEGDFRVGLGTDLTMYPPVYRESLGNGSGGWEFDFTVCGSTACRFVDSNGDVKEDDKAKEMWWQEINFNDINQDLYSRNDSDWVGSTPADTQPEFDYTEFALARDGVTLALTALNPAMGSLALGATYFLSDMVNWIASQHEDDSSLKRKWDYDGLSGPLYADSSTYLLARDEMTSNSYESFTIDNIAVAFPEFPVRTKYYVTFTAPDDPSTQSISTLEEEGIYRVPATEVAAARPPGSRRSKSAADEMVYVADPKKFIEVEPVKNPSIPDRIYEEIEQKKKQRSRKQ

SEQ ID NO: 141 - DNA, 1080 bp, Haloferax mediterranei
atgtcgaaagacagagatgggagaaggacaagtcggcgaggcacgttaaagaaaatcggcggtttcagtctcggagcgcttagtttcggggcagtcggacgaactcaagcggcgaccggctcatcggttacgaccgctgatatcgcacctcccggaccgaacggagacccgaagagtgttcagatagatgataaatacaccggagccgagatgtacggcgagggtgacttcagagtcggtctcggaactgacctgacgatgtatccgcccgtgtaccgtgagagtcttggaaatggaagcgggggttgggaattcgacttcaccgtttgtgggtccactgcctgtcgatttgtggacagtaacggtgacgtcaaagaggacgacaaggcgaaagaaatgtggtggcaggaaattaacttcaacgacataaatcaggatttatacagtcggaacgattccgactgggtcgggtcgacccctgccgatacccaaccggagttcgattacaccgactttgcgctcgctcgggacggagtgacgctcgctctcacggcactcaaccccgcaatggggagtcttgcactcggtgccacgtacttcctcagcgacatggtgaactggattgcgagccagcacgaagacgacagttcgctcaagagaaaatgggattacgacgggctaagtgggccgttgtacgccgattcgtcgacgtacctactggcacgcgacgagatgacttcgaactcgtacgaatcattcacgatcgataacatcgccgttgccttcccagagttccccgtccggaccaagtactacgtcacattcactgcgccggatgacccgtcaacgcagtcgatatctacgctcgaagaggagggaatctaccgagtgcccgctacggaagtggctgcggccagaccaccggggtcccgacgttccaaatcggcagccgacgagatggtgtacgttgccgatccgaagaagttcatagaggtcgagccggtgaagaacccaagtatcccggaccgaatctacgaggagatagagcaaaaaaagaaacaacggagtaggaaacagtag

SEQ ID NO: 142 - protein, 311 aa, Haloarchaeon S8a
MSDKDSINRRNVLRKIGGIGVASAVGFSGLASGESLSDDEKQDVIDTIYKSQRVEQIKKKFGGVNIEPKKVQSVTTNQSGDLVTAKLSVSDGDLVYSSVKDTTVIVQFDRSASEIGESWPKNTEAFIKSTSSGVDLLRTATDEEIKDVTEGVNTSEIESADAVNIFIDPESQTYYMEKYDFNNKVLEMFELATGGTSSGKISPTREDQNHEYNVREHKVFNSEKQNIQLQSDCNINSNTAADVILCFNQVGSCALCSPTLVGGPVPTVACLLVVCFGTPNAVSAILEEVDNSCFNLIKDVISCWDEWTSFW

SEQ ID NO: 143 - DNA, 936 bp, Haloarchaeon S8a
atgtcggataaagacagcattaacagaagaaatgtattaagaaaaattggcggtatcggtgtggcttcagctgtcggattttctggtttggcaagcggggaaagtcttagcgatgatgagaaacaagatgttattgacacaatttacaaatcacaaagagttgaacagataaagaaaaagttcggaggagtgaatattgagccgaaaaaggttcaatctgtaacgaccaatcagagcggagatcttgttacggcgaagctgtcggttagtgatggggatttggtatattcgagtgtcaaagatacaactgtaatagttcagttcgatagatcggcttctgaaattggtgaaagttggcccaagaatactgaggcattcatcaaatcgacgtcctctggggtcgatcttctacgtacagcaactgatgaagaaataaaggacgttactgagggagtcaacacatctgaaattgaatctgcggatgctgttaacatatttattgatcctgaatcacagacatactatatggagaaatatgactttaataataaggtacttgagatgtttgaattagcgacaggtgggacaagtagtggtaaaatctcccccacacgtgaagaccagaatcacgaatataatgttagggaacataaagtatttaactcagaaaaacagaatatacaacttcagagtgactgtaatataaacagtaacaccgctgctgatgttattctatgcttcaaccaggttggttcttgtgcactctgctccccgactttagtcggaggtccagtccctacagttgcatgtctcttagtcgtctgtttcggcactccaaatgctgtgtccgcgatacttgaagaagtcgataattcttgctttaacttgatcaaggatgtaatttcgtgttgggatgaatggactagcttctggtga

SEQ ID NO: 144 - protein, 333 aa, Lactobacillus helveticus
MKHLNETTNVRILSQFDMDTGYQAVVQKGNVGSKYVYGLQLRKGATTILRGYRGSKINNPILELSGQAGGHTQTWEFAGDRKDINGEERAGQWFIGVKPSKIEGSKIIWAKQIARVDLRNQMGPHYSNTDFPRLSYLNRAGSNPFAGNKMTHAEAAVSPDYTKFLIATVENNCIGHFTIYNLDTINEKLDEKGNSEDVNLETVKYEDSFIIDNLYGDDNNSIVNSIQGYDLDNDGNIYISSQKAPDFDGSYYAHHKQIVKIPYYARSKESEDQWRAVNLSEFGGLDIPGKHSEVESIQIIGENHCYLTVAYHSKNKAGENKTTLNEIYELSWN

SEQ ID NO: 145 - DNA, 1002 bp, Lactobacillus helveticus
atgaagcatttaaatgaaacaactaatgttagaattttaagtcaatttgatatggatactggctatcaagcagtagttcaaaaaggcaatgtaggttcaaaatatgtatatggattacaacttcgcaaaggtgctactactatcttgcgtggttaccgtggaagtaaaattaataaccctattcttgaattatctggtcaagcaggtggtcacacacagacatgggaatttgctggtgatcgtaaagacattaatggtgaagaaagagcaggtcaatggtttataggtgttaaaccatcgaaaattgaaggaagcaaaattatttgggcaaagcaaattgcaagagttgatcttagaaatcaaatgggacctcattattcaaatactgactttcctcgattatcctacttgaatcgcgccggttctaatccatttgctggtaataagatgacgcatgccgaagccgcagtatcacctgattatactaagtttttaattgctactgttgaaaataactgtattggtcattttactatatacaatttagatacaattaatgaaaaacttgatgaaaagggaaatagtgaagatgttaatctcgaaactgttaaatacgaagatagttttatcattgataatttatatggtgatgataataattctattgtaaattcaattcaagggtatgatttggataatgatggaaatatttatatttccagtcaaaaagcgccagattttgatggctcttattatgcacatcataagcagattgttaagattccatattatgctcggtctaaagaaagcgaagaccaatggagagctgtaaatttaagcgaattcggtggcttggatattccaggtaaacatagtgaagttgaaagcatccaaattattggtgagaatcattgttacttaactgttgcatatcattctaaaaataaagcgggtgaaaataaaactactttgaatgagatttatgaattatcttggaattag

SEQ ID NO: 146 - protein, 74 aa, Enterococcus hirae; identical to SEQ ID NO: 20

SEQ ID NO: 147 - DNA, 225 bp, Enterococcus hirae; identical to SEQ ID NO: 21

SEQ ID NO: 148 - protein, 75 aa, Lactobacillus johnsonii
MKQFNYLSHKDLAVVVGGRNNWQTNVGGAVGSAMIGATVGGTICGPACAVAGAHYLPILWTAVTAATGGFGKIRK

SEQ ID NO: 149 - DNA, 228 bp, Lactobacillus johnsonii
atgaaacaatttaattatttatcacataaagatttagcagtcgttgttggtggaagaaataattggcaaacaaatgtgggaggagcagtgggatcagctatgattggggctacagttggtggtacaatttgtggacctgcatgtgctgtagctggtgcccattatcttcctattttatggacagcggttacagctgcaacaggtggttttggcaagataagaaagtag

SEQ ID NO: 150 - protein, 62 aa, Lactobacillus johnsonii
MKLNDKELSKIVGGNRWGDTVLSAASGAGTGIKACKSFGPWGMAICGVGGAAIGGYFGYTHN

SEQ ID NO: 151 - DNA, 189 bp, Lactobacillus johnsonii
atgaaattaaatgacaaagaattatcaaagattgttggtggaaatcgatggggagatactgttttatcagctgctagtggcgcaggaactggtattaaagcatgtaaaagttttggcccatggggaatggcaatttgtggtgtaggaggtgcagcaataggaggttattttggctatactcataattaa

SEQ ID NO: 152 - protein, 59 aa, Lactococcus lactis subsp. lactis
MNKNEIETQPVTWLEEVSDQNFDEDVFGACSTNTFSLSDYWGNNGAWCTLTHECMAWCK

SEQ ID NO: 153 - DNA, 180 bp, Lactococcus lactis subsp. lactis
atgaacaaaaatgaaattgaaacacaaccagttacatggttggaagaagtatctgatcaaaattttgatgaagatgtatttggtgcgtgtagtactaacacattctcgctcagtgattactggggaaataacggggcttggtgtacactcactcatgaatgtatggcttggtgtaaataa

SEQ ID NO: 154 - protein, 65 aa, Lactococcus lactis subsp. lactis
MKEKNMKKNDTIELQLGKYLEDDMIELAEGDESHGGTTPATPAISILSAYISTNTCPTTKCTRAC

SEQ ID NO: 155 - DNA, 198 bp, Lactococcus lactis subsp. lactis
atgaaagaaaaaaatatgaaaaagaatgacactattgaattacaattgggaaaataccttgaagatgatatgattgaattagctgaaggggatgagtctcatggaggaacaacaccagcaactcctgcaatctctattctcagtgcatatattagtaccaatacttgtccaacaacaaaatgtacacgtgcttgttaa

SEQ ID NO: 156 - protein, 51 aa, Lactococcus lactis subsp. lactis
MKEQNSFNLLQEVTESELDLILGAKGGSGVIHTISHECNMNSWQFVFTCCS

SEQ ID NO: 157 - DNA, 156 bp, Lactococcus lactis subsp. lactis
atgaaagaacaaaactcttttaatcttcttcaagaagtgacagaaagtgaattggaccttattttaggtgcaaaaggcggcagtggagttattcatacaatttctcatgaatgtaatatgaatagctggcaatttgtatttacttgctgctcttaa

SEQ ID NO: 158 - protein, 53 aa, Lactococcus lactis
MAGFLKVVQLLAKYGSKAVQWAWANKGKILDWLNAGQAIDWVVSKIKQILGIK

SEQ ID NO: 159 - DNA, 162 bp, Lactococcus lactis
atggcagggtttttaaaagtagttcaattactagctaaatatggttctaaagctgtacaatgggcttgggcaaacaagggtaagattttagattggcttaatgcaggtcaggctattgattgggtagtttcgaaaattaagcaaattttaggtattaagtaa

SEQ ID NO: 160 - protein, 53 aa, Lactococcus lactis
MAGFLKVVQILAKYGSKAVQWAWANKGKILDWINAGQAIDWVVEKIKQILGIK

SEQ ID NO: 161 - DNA, 162 bp, Lactococcus lactis
atggcagggtttttaaaagtagtccaaattttggctaagtatggttctaaagccgtacaatgggcatgggcaaataaaggaaaaatcttagattggattaatgcaggtcaagctattgactgggtagttgaaaagattaagcaaattttgggtattaaataa

SEQ ID NO: 162 - protein, 65 aa, Lactobacillus amylovorus
MKQLNSEQLQNIIGGNRWTNAYSAALGCAVPGVKYGKKLGGVWGAVIGGVGGAAVCGLAGYVRKG

SEQ ID NO: 163 - DNA, 198 bp, Lactobacillus amylovorus
atgaaacaattgaattcagaacaattacaaaatattatcggtggaaatagatggactaatgcatacagcgcagctttgggatgcgctgtccctggagttaaatatggaaaaaaacttggtggcgtatggggtgctgtaattggtggcgtaggcggtgcagcagtctgtggcttggcgggttatgttcgtaaaggctaa

SEQ ID NO: 164 - protein, 68 aa, Lactobacillus sakei L45
MKTEKKVLDELSLHASAKMGARDVESSMNADSTPVLASVAVSMELLPTASVLYSDVAGCFKYSAKHHC

SEQ ID NO: 165 - DNA, 207 bp, Lactobacillus sakei L45
atgaaaacagaaaaaaaggttttagatgaactgagcttacacgcttctgcaaaaatgggagcacgtgatgttgaatccagcatgaatgcagactcaacaccagttttagcatcagtcgctgtatccatggaattattgccaactgcgtctgttctttattcggatgttgcaggttgcttcaaatattctgcaaaacatcattgttag

SEQ ID NO: 166 - protein, 91 aa, Lactococcus lactis subsp. lactis
MKTKSLVLALSAVTLFSAGGIVAQAEGTWQHGYGVSSAYSNYHHGSKTHSATVVNNNTGRQGKDTQRAGVWAKATVGRNLTEKASFYYNFW

SEQ ID NO: 167 - DNA, 276 bp, Lactococcus lactis subsp. lactis
atgaaaaccaagtctctcgtattggcattatctgcggttacgttattctctgccggaggaattgtagctcaagctgaaggaacatggcaacatggatatggtgttagttcggcatattcaaattatcatcatggtagcaaaactcattcagccacagttgtaaataataatactggccgacaaggtaaggatacacaacgtgccggtgtttgggcaaaagctactgttggacgtaacttaactgaaaaagcttcattttattataacttttggtaa

SEQ ID NO: 168 - protein, 75 aa, Lactococcus lactis subsp. cremoris
MKNQLNFNIVSDEELSEANGGKLTFIQSTAAGDLYYNTNTHKYVYQQTQNAFGAAANTIVNGWMGGAAGGFGLHH

SEQ ID NO: 169 - DNA, 228 bp, Lactococcus lactis subsp. cremoris
atgaaaaatcaattaaattttaatattgtttcagatgaagaactttcagaagctaacggaggaaaattaacatttattcaatcgacagcggctggagatttatattacaatactaatacacacaaatatgtttaccaacaaactcaaaacgcttttggggctgctgctaataccattgttaatggatggatgggtggcgctgctggaggtttcgggttgcaccattga

SEQ ID NO: 170 - protein, 68 aa, Lactococcus lactis subsp. cremoris
MKNQLNFNIVSDEELAEVNGGSLQYVMSAGPYTWYKDTRTGKTICKQTIDTASYTFGVMAEGWGKTFH

SEQ ID NO: 171 - DNA, 207 bp, Lactococcus lactis subsp. cremoris
atgaaaaatcaattaaattttaatattgtttctgatgaagaacttgcagaagttaatggaggaagcttgcagtatgttatgagtgctggaccatatacttggtataaagatactagaacaggaaaaacaatatgtaaacagacaattgacacagcaagttatacatttggtgtaatggcagaaggatggggaaaaacattccactaa

SEQ ID NO: 172 - protein, 63 aa, Lactococcus sp. QU 12
MKLIDHLGAPRWAVDTILGAIAVGNLASWVLALVPGPGWAVKAGLATAAAIVKHQGKAAAAAW

SEQ ID NO: 173 - DNA, 192 bp, Lactococcus sp. QU 12
atgaaattaattgatcatttaggtgctccaagatgggccgttgatactattttaggtgcaatcgcagttgggaacttagcaagttgggttctagcgcttgtccctggtccagggtgggcagtaaaagctggtttagcaactgctgctgccatcgttaaacatcaaggtaaagctgccgctgctgcttggtaa

SEQ ID NO: 174 - protein, 50 aa, Brevibacillus sp. GI-9
MACQCPDAISGWTHTDYQCHGLENKMYRHVYAICMNGTQVYCRTEWGSSC

SEQ ID NO: 175 - DNA, 153 bp, Brevibacillus sp. GI-9
atggcttgccaatgtccagatgcgatctcaggttggacgcatacagattaccagtgtcacggtttggagaataaaatgtatagacatgtttatgcaatttgcatgaacggtactcaagtatattgcagaacagagtggggtagcagctgctag

SEQ ID NO: 176 - protein, 57 aa, Leuconostoc pseudomesenteroides
MNKEYNSISNFKKITNKDLQNINGGFIGRAIGDFVYFGAKGLRESGKLLNYYYKHKH

SEQ ID NO: 177 - DNA, 174 bp, Leuconostoc pseudomesenteroides
atgaataaagaatataatagcattagcaattttaaaaaaattactaataaagacttgcaaaacataaatggtggatttattggtagggcaataggtgactttgtgtactttggagcgaagggactaagagaatctggtaaactacttaattattactataagcataagcattga

SEQ ID NO: 178 - protein, 53 aa, Leuconostoc pseudomesenteroides
MKNQLMSFEVISEKELSTVQGGKGLGKLIGIDWLLGQAKDAVKQYKKDYKRWH

SEQ ID NO: 179 - DNA, 162 bp, Leuconostoc pseudomesenteroides
atgaaaaatcagttaatgtctttcgaagtgatatcagaaaaagaattgtccacggtacaaggtggcaaaggcttaggtaaactcataggaattgattggcttttgggtcaagctaaggacgctgttaaacagtacaagaaggattacaaacgttggcactaa

SEQ ID NO: 180 - protein, 61 aa, Leuconostoc gelidum
MMNMKPTESYEQLDNSALEQVVGGKYYGNGVHCTKSGCSVNWGEAFSAGVHRLANGGNGFW

SEQ ID NO: 181 - DNA, 186 bp, Leuconostoc gelidum
atgatgaacatgaaacctacggaaagctatgagcaattggataatagtgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 182 - protein, 61 aa, Leuconostoc carnosum
MNNMKSADNYQQLDNNALEQVVGGKYYGNGVHCTKSGCSVNWGEAFSAGVHRLANGGNGFW

SEQ ID NO: 183 - DNA, 186 bp, Leuconostoc carnosum
atgaataacatgaaatctgcggataattatcagcaattggataataatgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 184 - protein, 63 aa, Leuconostoc mesenteroides
MFLVNQLGISKSLANTILGAIAVGNLASWLLALVPGPGWATKAALATAETIVKHEGKAAAIAW

SEQ ID NO: 185 - DNA, 192 bp, Leuconostoc mesenteroides
atgttcttggtaaatcagttagggatttcaaaatcgttagctaatactattcttggtgcaattgctgttggtaatttggccagttggttattagctttggttcctggtccgggttgggcaacaaaagcagcacttgcgacagctgaaacaattgtgaagcatgaaggaaaagcagctgctattgcgtggtaa

SEQ ID NO: 186 - protein, 74 aa, Bacillus licheniformis
MSKKEMILSWKNPMYRTESSYHPAGNILKELQEEEQHSIAGGTITLSTCAILSKPLGNNGYLCTVTKECMPSCN

SEQ ID NO: 187 - DNA, 225 bp, Bacillus licheniformis
atgtcaaaaaaggaaatgattctttcatggaaaaatcctatgtatcgcactgaatcttcttatcatccagcagggaacatccttaaagaactccaggaagaggaacagcacagcatcgccggaggcacaatcacgctcagcacttgtgccatcttgagcaagccgttaggaaataacggatacctgtgtacagtgacaaaagaatgcatgccaagctgtaactaa

SEQ ID NO: 188 - protein, 266 aa, Brevibacterium linens
MNNLYRELAPIPGPAWAEIEEEARRTFKRNIAGRRIVDVAGPTGFETSAVTTGHIRDVQSETSGLQVKQRIVQEYIELRTPFTVTRQAIDDVARGSGDSDWQPVKDAATTIAMAEDRAILHGLDAAGIGGIVPGSSNAAVAIPDAVEDFADAVAQALSVLRTVGVDGPYSLLLSSAEYTKVSESTDHGYPIREHLSRQLGAGEIIWAPALEGALLVSTRGGDYELHLGQDLSIGYYSHDSETVELYLQETFGFLALTDESSVPLSL

SEQ ID NO: 189 - DNA, 801 bp, Brevibacterium linens
gtgaataacctctatcgcgagcttgcccccatccccggcccggcctgggcggagatcgaggaggaggctcgacggacattcaaacgcaatatcgccggccgccggatcgtcgatgtcgcagggcccacgggcttcgagacctccgcggtgaccactggccacatccgagacgtccagtcggagacgagcggactgcaggttaagcagcgcatcgtgcaggaatacatcgagctgcggaccccattcaccgtgactcggcaggccatcgatgacgtggcccgcgggtccggtgactcggactggcagcccgtcaaggatgcggccacgacgatcgcgatggctgaagatcgggccattctccacgggctcgatgcggccgggatcggcggaatcgttcccggcagctcgaatgccgcagtggccatccccgacgccgtcgaggacttcgcggacgccgtcgcccaggcgctgagtgtgctgcgcacggtgggagtcgacgggccctacagcctgttgctctcctccgcggagtacaccaaggtctccgagtccaccgaccacggctacccgatccgcgagcacctctcccggcagctcggcgccggagagatcatctgggcgcccgcgctcgaaggggcgctgctcgtctccacgcgcgggggtgactacgagctccacctcggccaggacctgtcgatcggttactacagccacgacagcgagaccgtcgaactctatctgcaggagaccttcggattcctcgcgctgaccgacgaatccagtgtgcctttgagcctctga

SEQ ID NO: 190 - protein, 71 aa, Listeria innocua
MKKAALKFIIVIAILGFSFSFFSIQSEAKSYGNGVQCNKKKCWVDWGSAISTIGNNSAANWATGGAAGWKS

SEQ ID NO: 191 - DNA, 216 bp, Listeria innocua
ttgaagaaggcagcgttaaaatttattattgttattgctattctaggtttcagtttttctttctttagcatacaatctgaagctaaatcttatggaaatggagttcagtgtaataagaaaaaatgttgggtagattggggtagtgctataagtactattggaaataattctgcagcgaattgggctacaggtggagcagctggttggaaaagctga

SEQ ID NO: 192 - protein, 68 aa, Bacillus sp.
MSQEAIIRSWKDPFSRENSTQNPAGNPFSELKEAQMDKLVGAGDMEAACTFTLPGGGGVCTLTSECIC

SEQ ID NO: 193 - DNA, 207 bp, Bacillus sp.
atgagtcaagaagctatcattcgttcatggaaagatcctttttcccgtgaaaattctacacaaaatccagctggtaacccattcagtgagctgaaagaagcacaaatggataagttagtaggtgcgggagacatggaagcagcatgtacttttacattgcctggtggcggcggtgtttgtactctaacttctgaatgtatttgttaa

SEQ ID NO: 194 - protein, 61 aa, Leuconostoc mesenteroides
MTNMKSVEAYQQLDNQNLKKVVGGKYYGNGVHCTKSGCSVNWGEAASAGIHRLANGGNGFW

SEQ ID NO: 195 - DNA, 186 bp, Leuconostoc mesenteroides
atgacgaatatgaagtctgtggaagcatatcagcaattagataaccagaatctcaagaaagttgttggtggaaagtattatgggaatggtgttcactgtacaaaaagtggatgctctgttaactggggagaagctgcctcagctggcatacatcgtttggccaatggtggaaatggattttggtaa

SEQ ID NO: 196 - protein, 68 aa, Clavibacter michiganensis subsp. michiganensis
MNDILETETPVMVSPRWDMLLDAGEDTSPSVQTQIDAEFRRVVSPYMSSSGWLCTLTIECGTIICACR

SEQ ID NO: 197 - DNA, 207 bp, Clavibacter michiganensis subsp. michiganensis
atgaacgacatcctcgagacggagacccccgtcatggtcagcccccggtgggacatgctgctcgacgcgggcgaggacaccagcccgtccgtccagacccagatcgacgcggagttccgtcgcgtcgtgagcccgtacatgtccagcagcggctggctctgcacgctcaccatcgaatgtggcaccatcatctgcgcgtgtcgctga

SEQ ID NO: 198 - protein, 69 aa, Escherichia coli
MELKASEFGVVLSVDALKLSRQSPLGVGIGGGGGGGGGGSCGGQGGGCGGCSNGCSGGNGGSGGSGSHI

SEQ ID NO: 199 - DNA, 207 bp, Escherichia coli
atggaattaaaagcgagtgaatttggtgtagttttgtccgttgatgctcttaaattatcacgccagtctccattaggtgttggcattggtggtggtggcggcggcggcggcggcggtagctgcggtggtcaaggtggcggttgtggtggttgcagcaacggttgtagtggtggaaacggtggcagcggcggaagtggttcacatatc

SEQ ID NO: 200 - protein, 7 aa, Escherichia coli
MRTGNAN

SEQ ID NO: 201 - DNA, 24 bp, Escherichia coli
atgcgtactggtaatgcaaactaa

SEQ ID NO: 202 - protein, 99 aa, Klebsiella pneumoniae
MREISQKDLNLAFGAGETDPNTQLLNDLGNNMAWGAALGAPGGLGSAALGAAGGALQTVGQGLIDHGPVNVPIPVLIGPSWNGSGSGYNSATSSSGSGS

SEQ ID NO: 203 - DNA, 300 bp, Klebsiella pneumoniae
atgagagaaattagtcaaaaggacttaaatcttgcttttggtgcaggagagaccgatccaaatactcaacttctaaacgaccttggaaataatatggcatggggtgctgctcttggcgctcctggcggattaggatcagcagctttgggggccgcgggaggtgcattacaaactgtagggcaaggattaattgaccatggtcctgtaaatgtccccatccctgtactcatcgggccaagctggaatggtagcggtagtggttataacagcgcaacatccagttccggtagtggtagttaa

SEQ ID NO: 204 - protein, 75 aa, Escherichia coli
MREITESQLRYISGAGGAPATSANAAGAAAIVGALAGIPGGPLGVVVGAVSAGLTTAIGSTVGSGSASSSAGGGS

SEQ ID NO: 205 - DNA, 228 bp, Escherichia coli
atgcgagaaataacagaatcacagttaagatatatttccggggcgggaggtgcgccagcgacttcagctaatgccgcaggtgctgcagctattgttggagctctcgccggaatacctggtggtccacttggggttgtagttggagccgtatctgccggtttgacaacagcaattggctcgaccgtgggaagtggtagtgccagttcttctgctggtggcggtagctaa

SEQ ID NO: 206 - protein, 58 aa, Escherichia coli
MIKHFHFNKLSSGKKNNVPSPAKGVIQIKKSASQLTKGGAGHVPEYFVGIGTPISFYG

SEQ ID NO: 207 - DNA, 177 bp, Escherichia coli
atgattaagcattttcattttaataaactgtcttctggtaaaaaaaataatgttccatctcctgcaaagggggttatacaaataaaaaaatcagcatcgcaactcacaaaaggtggtgcaggacatgtgcctgagtattttgtggggattggtacacctatatctttctatggctga

SEQ ID NO: 208 - protein, 90 aa, Escherichia coli
MYMRELDREELNCVGGAGDPLADPNSQIVRQIMSNAAWGPPLVPERFRGMAVGAAGGVTQTVLQGAAAHMPVNVPIPKVPMGPSWNGSKG

SEQ ID NO: 209 - DNA, 273 bp, Escherichia coli
atgtatatgagagagttagatagagaggaattaaattgcgttggtggggctggagatccgcttgcagatcctaattcccaaattgtaagacagataatgtctaatgcggcatggggcccgcctttggtgccagagcggtttaggggaatggctgttggagccgcaggtggggttacgcagacagttcttcaaggagcagcagctcatatgccggttaatgtccctatacctaaagttccgatgggaccctcatggaacggaagtaaaggataa

SEQ ID NO: 210 - protein, 50 aa, Enterococcus mundtii
MSQVVGGKYYGNGVSCNKKGCSVDWGKAIGIIGNNSAANLATGGAAGWKS

SEQ ID NO: 211 - DNA, 153 bp, Enterococcus mundtii
atgtcacaggtagtaggtggaaaatactacggtaatggagtctcatgtaataaaaaagggtgcagtgttgattggggaaaagcgattggcattattggaaataattctgctgcgaatttagctactggtggagcagctggttggaaaagttaa

SEQ ID NO: 212 - protein, 58 aa, Enterococcus mundtii
MKKLTSKEMAQVVGGKYYGNGLSCNKKGCSVDWGKAIGIIGNNSAANLATGGAAGWKS

SEQ ID NO: 213 - DNA, 177 bp, Enterococcus mundtii
ttgaagaaattaacatcaaaagaaatggcacaagtagtaggtgggaaatactacggtaatggattatcatgtaataaaaaagggtgcagtgttgattggggaaaagctattggcattattggaaataattctgctgcgaatttagctactggtggagcagctggttggaaaagttaa

SEQ ID NO: 214 - protein, 63 aa, Streptococcus mutans
MSNTQLLEVLGTETFDVQEDLFAFDTTDTTIVASNDDPDTRFKSWSLCTPGCARTGSFNSYCC

SEQ ID NO: 215 - DNA, 192 bp, Streptococcus mutans
atgtcaaacacacaattattagaagtccttggtactgaaacttttgatgttcaagaagatctctttgcttttgatacaacagatactactattgtggcaagcaacgacgatccagatactcgtttcaaaagttggagcctttgtacgcctggttgtgcaaggacaggtagtttcaatagttactgttgctga

SEQ ID NO: 216 - protein, 53 aa, Streptococcus mutans
MNKLNSNAVVSLNEVSDSELDTILGGNRWWQGVVPTVSYECRMNSWQHVFTCC

SEQ ID NO: 217 - DNA, 162 bp, Streptococcus mutans
atgaacaagttaaacagtaacgcagtagtttctttgaatgaagtttcagattctgaattggatactattttgggtggtaatcgttggtggcaaggtgttgtgccaacggtctcatatgagtgtcgcatgaattcatggcaacatgttttcacttgctgttaa

SEQ ID NO: 218 - protein, 57 aa, Lactococcus lactis subsp. lactis
MSTKDFNLDLVSVSKKDSGASPRITSISLCTPGCKTGALMGCNMKTATCHCSIHVSK

SEQ ID NO: 219 - DNA, 174 bp, Lactococcus lactis subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtcattgtagtattcacgtaagcaaataa

SEQ ID NO: 220 - protein, 57 aa, Lactococcus lactis
MSTKDFNLDLVSVSKKDSGASPRITSISLCTPGCKTGALMGCNMKTATCNCSVHVSK

SEQ ID NO: 221 - DNA, 171 bp, Lactococcus lactis
atgagtacaaaagatttcaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagcgttcacgtaagcaaa

SEQ ID NO: 222 - protein, 57 aa, Lactococcus lactis
MSTKDFNLDLVSVSKTDSGASTRITSISLCTPGCKTGVLMGCNLKTATCNCSVHVSK

SEQ ID NO: 223 - DNA, 174 bp, Lactococcus lactis
atgagtacaaaagatttcaacttagatttggtatctgtttcaaaaacagattctggcgcttcaacacgtattaccagcatttcgctttgtacaccaggttgtaaaacaggtgttctgatgggatgtaacctgaaaacagcaacttgtaattgtagcgttcacgtaagcaaataa

SEQ ID NO: 224 - protein, 55 aa, Streptococcus uberis
MNNEDFNLDLIKISKENNSGASPRITSKSLCTPGCKTGILMTCPLKTATCGCHFG

SEQ ID NO: 225 - DNA, 168 bp, Streptococcus uberis
atgaacaatgaagattttaatttggatctcatcaaaatctcaaaggaaaacaactcaggagcttcacctcgaataactagtaaatcattatgtactcctggatgtaagacgggtattttgatgacttgtccactaaaaactgcaacctgtggttgtcattttggataa

SEQ ID NO: 226 - protein, 57 aa, Lactococcus lactis subsp. lactis
MSTKDFNLDLVSVSKKDSGASPRITSISLCTPGCKTGALMGCNMKTATCNCSIHVSK

SEQ ID NO: 227 - DNA, 174 bp, Lactococcus lactis subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagtattcacgtaagcaaataa

SEQ ID NO: 228 - protein, 57 aa, Staphylococcus warneri
MENSKVMKDIEVANLLEEVQEDELNEVLGAKKKSGVIPTVSHDCHMNSFQFVFTCCS

SEQ ID NO: 229 - DNA, 174 bp, Staphylococcus warneri
atggaaaattctaaagttatgaaggacattgaagtagcaaatttattagaagaggttcaagaagatgaattgaatgaagtcttaggagctaagaaaaagtcaggagtaatcccaactgtgtcacacgattgccatatgaattctttccaatttgtatttacttgttgttcataa

SEQ ID NO: 230 - protein, 58 aa, Paenibacillus polymyxa
MAENLFDLDIQVNKSQGSVEPQVLSIVACSSGCGSGKTAASCVETCGNRCFTNVGSLC

SEQ ID NO: 231 - DNA, 177 bp, Paenibacillus polymyxa
atggctgaaaacttatttgatctggacattcaagtaaacaaatctcaaggttctgtagagcctcaggttctgagcattgttgcatgttctagcggatgtggtagcggtaaaacagctgccagttgtgttgaaacttgtggcaaccggtgctttactaacgttggttcactctgctaa

SEQ ID NO: 232 - protein, 62 aa, Pediococcus acidilactici
MKKIEKLTEKEMANIIGGKYYGNGVTCGKHSCSVDWGKATTCIINNGAMAWATGGHQGNHKC

SEQ ID NO: 233 - DNA, 189 bp, Pediococcus acidilactici
atgaaaaaaattgaaaaattaactgaaaaagaaatggccaatatcattggtggtaaatactacggtaatggggttacttgtggcaaacattcctgctctgttgactggggtaaggctaccacttgcataatcaataatggagctatggcatgggctactggtggacatcaaggtaatcataaatgctag

SEQ ID NO: 234 - protein, 60 aa, Pediococcus pentosaceus
MTEIKVLNDKELKNVVGGKYYGNGVHCGKKTCYVDWGQATASIGKIIVNGWTQHGPWAHR

SEQ ID NO: 235 - DNA, 183 bp, Pediococcus pentosaceus
atgactgaaattaaagtactaaacgataaggaactaaaaaatgtcgtaggaggaaagtattacggtaacggagtgcattgtggtaaaaagacttgctatgtggactggggacaagctacagctagcattggaaaaattatagtgaacggatggacacaacacgggccttgggcacatagataa

SEQ ID NO: 236 - protein, 60 aa, Staphylococcus epidermidis
MKNNKNLFDLEIKKETSQNTDELEPQTAGPAIRASVKQCQKTLKATRLFTVSCKGKNGCK

SEQ ID NO: 237 - DNA, 183 bp, Staphylococcus epidermidis
atgaaaaataacaaaaatttatttgatttagaaattaaaaaagaaacaagtcaaaacactgatgaacttgaacctcaaactgctggaccagcgattagagcttctgtgaaacaatgtcagaaaactttgaaagctacgcgtttatttacagtgtcttgcaaaggaaaaaacggatgtaaatag

SEQ ID NO: 238 - protein, 62 aa, Carnobacterium maltaromaticum
MKTVKELSVKEMQLTTGGKYYGNGVSCNKNGCTVDWSKAIGIIGNNAAANLTTGGAAGWNKG

SEQ ID NO: 239 - DNA, 189 bp, Carnobacterium maltaromaticum
atgaaaactgttaaagaacttagcgttaaagaaatgcaactaactacaggaggtaagtattacggaaatggcgtttcctgtaataaaaatggttgtactgtagattggagcaaagctattgggattataggaaacaatgcagcagcaaatttgactacaggtggagccgctggttggaacaaaggataa

SEQ ID NO: 240 - protein, 69 aa, Lactobacillus plantarum
  VARIANT 40: Xaa = any amino acid
MYKELTVDELALIDGGKKKKKKVACTWGNAATAAASGAVXGILGGPTGALAGAIWGVSQCASNNLHGMH

SEQ ID NO: 241 - DNA, 210 bp, Lactobacillus plantarum
  misc_feature 119: n = a, c, t or g
atgtataaagaattaacagttgatgaattagcattgattgatggaggaaaaaagaagaagaaaaaagtagcttgtacttggggaaatgcagcaacagccgctgcttctggtgcagttangggtattcttggtgggcctactggtgcactggctggagctatctggggcgtttcacaatgcgcgtctaacaacttacacggcatgcactaa

SEQ ID NO: 242 - protein, 56 aa, Lactobacillus plantarum
MMKKIEKLTEKEMANIIGGKYYGNGVTCGKHSCSVNWGQAFSCSVSHLANFGHGKC

SEQ ID NO: 243 - DNA, 171 bp, Lactobacillus plantarum
atgatgaaaaaaattgaaaaattaactgaaaaagaaatggccaatatcattggtggtaaatactatggtaatggggttacttgtggtaaacattcctgctctgttaactggggccaagcattttcttgtagtgtgtcacatttagctaacttcggtcatggaaagtgctaa

SEQ ID NO: 244 - protein, 64 aa, Lactobacillus plantarum
MSKLVKTLTVDEISKIQTNGGKPAWCWYTLAMCGAGYDSGTCDYMYSHCFGVKHSSGGGGSYHC

SEQ ID NO: 245 - DNA, 195 bp, Lactobacillus plantarum
atgagtaaactagttaaaacattaactgtcgatgaaatctctaagattcaaaccaatggtggaaaacctgcatggtgttggtacacattggcaatgtgcggtgctggttatgattcaggcacttgtgattatatgtattcacactgctttggtgtaaaacactctagcggtggtggcggtagctaccattgttag

SEQ ID NO: 246 - protein, 56 aa, Lactobacillus plantarum
MLQFEKLQYSRLPQKKLAKISGGFNRGGYNFGKSVRHVVDAIGSVAGIRGILKSIR

SEQ ID NO: 247 - DNA, 171 bp, Lactobacillus plantarum
atgctacagtttgagaaattacaatattccaggttgccgcaaaaaaagcttgccaaaatatctggtggttttaatcggggcggttataactttggtaaaagtgttcgacatgttgttgatgcaattggttcagttgcaggcattcgtggtattttgaaaagtattcgttaa

SEQ ID NO: 248 - protein, 52 aa, Lactobacillus plantarum
MKKFLVLRDRELNAISGGVFHAYSARGVRNNYKSAVGPADWVISAVRGFIHG

SEQ ID NO: 249 - DNA, 159 bp, Lactobacillus plantarum
atgaaaaaatttctagttttgcgtgaccgtgaattaaatgctatttcaggtggcgttttccatgcctatagcgcgcgtggcgttcggaataattataaaagtgctgttgggcctgccgattgggtcattagcgctgtccgaggattcatccacggatag

SEQ ID NO: 250 - protein, 55 aa, Lactobacillus plantarum
MTVNKMIKDLDVVDAFAPISNNKLNGVVGGGAWKNFWSSLRKGFYDGEAGRAIRR

SEQ ID NO: 251 - DNA, 168 bp, Lactobacillus plantarum
atgactgtgaacaaaatgattaaggatttggatgtagtagatgcatttgcacctatttctaataataagttgaacggggttgttgggggaggcgcttggaaaaatttctggtctagtttaagaaaaggattttatgatggcgaagctggcagagcaatccgtcgttaa

SEQ ID NO: 252 - protein, 57 aa, Lactobacillus plantarum
MKIKLTVLNEFEELTADAEKNISGGRRSRKNGIGYAIGYAFGAVERAVLGGSRDYNK

SEQ ID NO: 253 - DNA, 174 bp, Lactobacillus plantarum
atgaaaattaaattaactgttttaaatgaatttgaagaattaactgctgacgctgaaaagaatatttctggtggccgtcggagtcgtaaaaatggaattggatacgctattggttatgcgtttggcgcggttgaacgggccgtgcttggtggttcaagggattataataagtga

SEQ ID NO: 254 - protein, 47 aa, Lactobacillus plantarum
MDKFEKISTSNLEKISGGDLTTKLWSSWGYYLGKKARWNLKHPYVQF

SEQ ID NO: 255 - DNA, 141 bp, Lactobacillus plantarum
atggataaatttgaaaaaattagtacatctaacctagaaaagatctctggcggtgatttaacaaccaagttatggagctcttggggatattatcttggcaagaaagcacgttggaatttaaagcacccatatgttcaattt

SEQ ID NO: 256 - protein, 55 aa, Lactobacillus plantarum
MNNLNKFSTLGKSSLSQIEGGSVPTSVYTLGIKILWSAYKHRKTIEKSFNKGFYH

SEQ ID NO: 257 - DNA, 168 bp, Lactobacillus plantarum
atgaataacttgaataaattttctactctaggcaagagtagcttgtctcaaattgagggcggatcagtcccaacttcagtatatacgcttggaattaaaattctatggtctgcgtataagcatcgcaaaacgattgaaaaaagttttaataaaggcttttatcattaa

SEQ ID NO: 258 - protein, 55 aa, Lactobacillus plantarum
MNNALSFEQQFTDFSTLSDSELESVEGGRNKLAYNMGHYAGKATIFGLAAWALLA

SEQ ID NO: 259 - DNA, 168 bp, Lactobacillus plantarum
atgaataacgcattaagttttgaacaacaatttacagacttcagcaccttatcggactctgaattagaatccgttgagggtggccgaaataagcttgcatataatatggggcattacgctggtaaggcaaccatttttggacttgcagcatgggcactccttgcatga

SEQ ID NO: 260 - protein, 47 aa, Lactobacillus plantarum
MDKIIKFQGISDDQLNAVIGGKKKKQSWYAAAGDAIVSFGEGFLNAW

SEQ ID NO: 261 - DNA, 144 bp, Lactobacillus plantarum
atggataagattattaagtttcaagggatttctgatgatcaattaaatgctgttatcggtgggaaaaagaaaaaacaatcttggtacgcagcagctggtgatgcaatcgttagttttggtgaaggatttttaaatgcttggtaa

SEQ ID NO: 262 - protein, 59 aa, Lactobacillus plantarum
MKISKIEAQARKDFFKKIDTNSNLLNVNGAKCKWWNISCDLGNNGHVCTLSHECQVSCN

SEQ ID NO: 263 - DNA, 180 bp, Lactobacillus plantarum
atgaaaatttctaagattgaagctcaggctcgtaaagatttttttaaaaaaatcgatactaactcgaacttattaaatgtaaatggtgccaaatgcaagtggtggaatatttcgtgtgatttaggaaataatggccatgtttgtaccttgtcacatgaatgccaagtatcttgtaactaa

SEQ ID NO: 264 - protein, 67 aa, Lactobacillus plantarum
MTKTSRRKNAIANYLEPVDEKSINESFGAGDPEARSGIPCTIGAAVAASIAVCPTTKCSKRCGKRKK

SEQ ID NO: 265 - DNA, 204 bp, Lactobacillus plantarum
atgactaaaactagtcgtcgtaagaatgctattgctaattatttagaaccagtcgacgaaaaaagtattaatgaatcttttggggctggggatccggaagcaagatccggaattccatgtacaatcggcgcagctgtcgcagcatcaattgcagtttgtccaactactaagtgtagtaaacgttgtggcaagcgtaagaaataa

SEQ ID NO: 266 - protein, 48 aa, Lactobacillus plantarum
MKIQIKGMKQLSNKEMQKIVGGKSSAYSLQMGATAIKQVKKLFKKWGW

SEQ ID NO: 267 - DNA, 147 bp, Lactobacillus plantarum
atgaaaattcaaattaaaggtatgaagcaacttagtaataaggaaatgcaaaaaatagtaggtggaaagagtagtgcgtattctttgcagatgggggcaactgcaattaaacaggtaaagaaactgtttaaaaaatggggatggtaa

SEQ ID NO: 268 - protein, 207 aa, Propionibacterium jensenii
MNKTHKMATLVIAAILAAGMTAPTAYADSPGNTRITASEQSVLTQILGHKPTQTEYNRYVETYGSVPTEADINAYIEASESEGSSSQTAAHDDSTSPGTSTEIYTQAAPARFSMFFLSGTWITRSGVVSLSLKPRKGGIGNEGDERTWKTVYDKFHNAGQWTRYKNNGVDASMKKQYMCHFKYGMVKTPWNLEPHKKAADVSPVKCN

SEQ ID NO: 269 - DNA, 624 bp, Propionibacterium jensenii
atgaacaaaacacacaaaatggcgacgctggtaattgccgcgatcttggccgccggaatgaccgcaccaactgcctatgcagattctcctggaaacaccagaattacagccagcgagcaaagcgtccttacccagatactcggccacaaacctacacaaactgaatataaccgatacgttgagacttacggaagcgtaccgaccgaagcagacatcaacgcatatatagaagcgtctgaatctgagggatcatcaagtcaaacggctgctcacgatgactcgacatcacccggcacgagtaccgaaatctacacgcaggcagcccctgccaggttctcaatgtttttcctgtccggaacttggatcactaggagtggtgtagtatcgctctccttgaagccaaggaagggtggtattggcaacgagggggacgagcgtacctggaagactgtatacgacaaattccataacgctgggcaatggacacgatacaagaacaacggcgtagacgccagcatgaaaaagcagtacatgtgccacttcaagtacgggatggtgaagacgccatggaatctggagccccacaagaaggctgcagacgtcagtccagtcaagtgcaactag

SEQ ID NO: 270 - protein, 96 aa, Propionibacterium thoenii
MKKTLLRSGTIALATAAAFGASLAAAPSAMAVPGGCTYTRSNRDVIGTCKTGSGQFRIRLDCNNAPDKTSVWAKPKVMVSVHCLVGQPRSISFETK

SEQ ID NO: 271 - DNA, 291 bp, Propionibacterium thoenii
atgaagaagaccctcctgcgaagtggaacgatcgcactggcgaccgcggctgcatttggcgcatcattggcagccgccccatctgccatggccgttcctggtggttgcacgtacacaagaagcaatcgcgacgtcatcggtacctgcaagactggaagcggccagttccgaatccgacttgactgcaacaacgctccagacaaaacttcagtctgggccaagcccaaggtaatggtgtcggttcactgtcttgttggtcaaccgaggtccatctcgttcgagaccaagtga

SEQ ID NO: 272 - protein, 255 aa, Propionibacterium freudenreichii subsp freudenreii
MNTKAVNLKSENTTKLVSYLTENQLDEFIRRIRIDGALVEEVSQNAKQALDNTGLNGWINTDCDEGLLSDFISKIASARWIPLAESIRPAVTDRDKYRVSCWFYQGMNIAIYANIGGVANIIGYTEAAVATLLGAVVAVAPVVPGTPTPPKDKSSQYKEVPLAVRLSETYHEEGVRGLFDELNYSESRMISTLRRASTDGVLINSWNDGQDTILLKKYNFQDLQLTVRSRIVGNQTIIEECKITDGRKTLSDETV

SEQ ID NO: 273 - DNA, 768 bp, Propionibacterium freudenreichii subsp freudenreii
atgaataccaaagctgtaaatctgaagtcagaaaacacgactaagttggtgagctaccttacggaaaatcaattggatgagtttattagaaggattcgcattgatggcgctcttgtggaagaggtcagtcaaaatgctaagcaggccttagataatactgggctcaatggctggataaatactgattgcgatgaaggccttctctctgatttcatttcaaagatagcaagtgctagatggattccattagctgagtcaattcgacctgcggtgactgacagggataagtatcgagtaagttgctggttctaccaggggatgaatatagcaatttacgcaaatatcggtggcgtggccaatattatcggctatacggaggccgcagtcgcaacactccttggtgcagttgtggcggtagctcctgtggtccctggaactccaacccctccaaaggacaagagttcgcaatataaggaggttccccttgccgttcgtctttccgaaacataccacgaagagggagtacgaggtctattcgacgagctgaactactccgagagccgtatgatctctactctaaggcgagcatcaaccgatggagtcctaattaattcttggaacgatgggcaggatacaattctgcttaagaagtacaatttccaagacttgcaactgactgtcaggagccgcattgttgggaatcaaacaataattgaagaatgcaaaatcactgatggtagaaaaactctttcagacgagactgtgtag

SEQ ID NO: 274 - protein, 618 aa, Pseudomonas aeruginosa
MARPIADLIHFNSTTVTASGDVYYGPGGGTGIGPIARPIEHGLDSSTENGWQEFESYADVGVDPRRYVPLQVKEKRREIELQFRDAEKKLEASVQAELDKADAALGPAKNLAPLDVINRSLTIVGNALQQKNQKLLLNQKKITSLGAKNFLTRTAEEIGEQAVREGNINGPEAYMRFLDREMEGLTAAYNVKLFTEAISSLQIRMNTLTAAKASIEAAAANKAREQAAAEAKRKAEEQARQQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRVADGGGVYNMGNLVAVTPKRHIEIHKGGK

SEQ ID NO: 275 - DNA, 1857 bp, Pseudomonas aeruginosa
atggcacgacccattgctgaccttatccacttcaactctacaactgtcacggcaagcggagacgtttattacggccctgggggaggtaccggcattggccccattgccagacctatagagcacggcttggattcgtccactgaaaatggctggcaagagtttgaaagttatgctgatgtgggcgttgaccccagacgctatgttcctcttcaggttaaagaaaaacgcagggagatcgagcttcagttccgagatgccgagaaaaaacttgaggcgtcggtacaagccgagctggataaggctgatgccgctcttggtccggcaaagaatcttgcaccattggacgtcatcaaccgcagtctgaccatcgttggaaacgccctccagcaaaagaatcaaaaactactgctgaatcagaagaagattaccagcctgggtgcaaagaatttccttacccgtacggcggaagagatcggtgaacaagcggtgcgagaaggcaatattaacgggcctgaagcctatatgcgcttcctcgacagggaaatggaaggtctcacggcagcttataacgtaaaactcttcaccgaagcgatcagtagtctccagatccgcatgaatacgttgaccgccgccaaagcaagtattgaggcggccgcagcaaacaaggcgcgtgaacaagcagcggctgaggccaaacgcaaagccgaagagcaggcccgccagcaagcggcgataagagctgccaatacctatgccatgccggccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgagtagcagatggaggcggcgtttacaatatggggaaccttgttgcagtaacgccaaaacgtcatatagaaatccacaagggagggaagtga

SEQ ID NO: 276 - protein, 689 aa, Pseudomonas aeruginosa
MAVNDYEPGSMVITHVQGGGRDIIQYIPARSSYGTPPFVPPGPSPYVGTGMQEYRKLRSTLDKSHSELKKNLKNETLKEVDELKSEAGLPGKAVSANDIRDEKSIVDALMDAKAKSLKAIEDRPANLYTASDFPQKSESMYQSQLLASRKFYGEFLDRHMSELAKAYSADIYKAQIAILKQTSQELENKARSLEAEAQRAAAEVEADYKARKANVEKKVQSELDQAGNALPQLTNPTPEQWLERATQLVTQAIANKKKLQTANNALIAKAPNALEKQKATYNADLLVDEIASLQARLDKLNAETARRKEIARQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRIADGGGVYNMGNLVAVTPKRHIEIHKGGK

SEQ ID NO: 277 - DNA, 2070 bp, Pseudomonas aeruginosa
atggctgtcaatgattacgaacctggttcgatggttattacacatgtgcagggtggtgggcgtgacataatccagtatattcctgctcgatcaagctacggtactccaccatttgtcccaccaggaccaagtccgtatgtcggtactggaatgcaggagtacaggaagctaagaagtacgcttgataagtcccattcagaactcaagaaaaacctgaaaaatgaaaccctgaaggaggttgatgaactcaagagtgaagcggggttgccaggtaaagcggtcagtgccaatgacatccgcgatgaaaagagtatcgttgatgcactcatggatgccaaagcaaaatcgctaaaggccattgaggatcgcccggccaatctttatacggcttcagactttcctcagaagtcagagtcgatgtaccagagtcagttgctggccagccgaaaattctatggagagttcctggatcgccatatgagtgagctggccaaagcgtacagcgccgatatctataaggcgcaaatcgctatcttgaaacaaacgtctcaagagctggagaataaagcccggtcattggaagcagaagcccagcgagccgctgctgaggtggaggcggactacaaggccaggaaggcaaatgtcgagaaaaaagtgcagtccgagcttgaccaggctgggaatgctttgcctcaactgaccaatccaacgccagagcagtggcttgaacgcgctactcaactggttacgcaggcgatcgccaataagaagaaattgcagactgcaaacaatgccttgattgccaaggcacccaatgcactggagaaacaaaaggcaacctacaacgccgatctcctagtggatgaaatcgccagcctgcaagcacggctggacaagctgaacgccgaaacggcaaggcgcaaggaaatcgctcgtcaagcggcgatcagggctgccaatacttatgccatgccagccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgaatagcagatggaggcggcgtttacaatatggggaaccttgttgcagtaacgccaaaacgtcatatagaaatccacaagggagggaagtga

SEQ ID NO: 278 - protein, 47 aa, Ruminococcus gnavus
MRNDVLTLTNPMEEKELEQILGGGNGVLKTISHECNMNTWQFLFTCC

SEQ ID NO: 279 - DNA, 144 bp, Ruminococcus gnavus
atgagaaatgacgtattaacattaacaaacccaatggaagagaacgaactggagcagatcttaggtggtggcaatggtgtgttaaaaacgattagccacgaatgcaatatgaacacatggcagttcctgtttacttgttgctaa

SEQ ID NO: 280 - protein, 55 aa, Lactobacillus sakei
MKNAKSLTIQEMKSITGGKYYGNGVSCNSHGCSVNWGQAWTCGVNHLANGGHGVC

SEQ ID NO: 281 - DNA, 168 bp, Lactobacillus sakei
atgaaaaacgcaaaaagcctaacaattcaagaaatgaaatctattacaggtggtaaatactatggtaatggcgttagctgtaactctcacggctgttcagtaaattgggggcaagcatggacttgtggagtaaaccatctagctaatggcggtcatggagtttgttaa

SEQ ID NO: 282 - protein, 59 aa, Lactobacillus sakei; identical to SEQ ID NO: 68

SEQ ID NO: 283 - DNA, 180 bp, Lactobacillus sakei; identical to SEQ ID NO: 69

SEQ ID NO: 284 - protein, 61 aa, Lactobacillus sakei
MEKFIELSLKEVTAITGGKYYGNGVHCGKHSCTVDWGTAIGNIGNNAAANWATGGNAGWNK

SEQ ID NO: 285 - DNA, 186 bp, Lactobacillus sakei
atggaaaagtttattgaattatctttaaaagaagtaacagcaattacaggtggaaaatattatggtaacggtgtacactgtggaaaacattcatgtaccgtagactggggaacagctattggaaatatcggaaataatgcagctgcaaactgggccacaggcggaaacgctggctggaataaataa

SEQ ID NO: 286 - protein, 56 aa, Streptococcus salivarius
MKSTNNQSIAEIAAVNSLQEVSMEELDQIIGAGNGVVLTLTHECNLATWTKKLKCC

SEQ ID NO: 287 - DNA, 171 bp, Streptococcus salivarius
atgaaatcaacaaataatcaaagtatcgcagaaattgcagcagtaaactcactacaagaagtaagtatggaggaactagaccaaattattggtgccggaaacggagtggttcttactcttactcatgaatgtaacctagcaacttggacaaaaaaactaaaatgttgctaa

SEQ ID NO: 288 - protein, 51 aa, Streptococcus pyogenes serotype M28
MSFMKNSKDILTNAIEEVSEKELMEVAGGKKGSGWFATITDDCPNSVFVCC

SEQ ID NO: 289 - DNA, 156 bp, Streptococcus pyogenes serotype M28
atgagttttatgaaaaattcaaaggatattttgactaatgctatcgaagaagtttctgaaaaagaacttatggaagtagctggtggtaaaaaaggttccggttggtttgcaactattactgatgactgtccgaactcagtattcgtttgttgttaa

SEQ ID NO: 290 - protein, 48 aa, Streptococcus salivarius
MKNSKDVLNNAIEEVSEKELMEVAGGKKGPGWIATITDDCPNSIFVCC

SEQ ID NO: 291 - DNA, 147 bp, Streptococcus salivarius
atgaaaaactcaaaagatgttttgaacaatgctatcgaagaggtttctgaaaaagaacttatggaagtagctggtggtaaaaaaggtccaggttggattgcaactattactgatgactgtccaaactcaatattcgtttgttgttaa

SEQ ID NO: 292 - protein, 48 aa, Streptococcus salivarius
MKNSKDILNNAIEEVSEKELMEVAGGKRGSGWIATITDDCPNSVFVCC

SEQ ID NO: 293 - DNA, 147 bp, Streptococcus salivarius
atgaaaaactcaaaagatattttgaacaatgctatcgaagaagtttctgaaaaagaacttatggaagtagctggtggtaaaagaggttcaggttggattgcaactattactgatgactgtccaaactcagtattcgtttgttgttaa

SEQ ID NO: 294 - protein, 62 aa, Staphylococcus aureus
MKSSFLEKDIEEQVTWFEEVSEQEFDDDIFGACSTNTFSLSDYWGNKGNWCTATHECMSWCK

SEQ ID NO: 295 - DNA, 189 bp, Staphylococcus aureus
atgaaaagttcttttttagaaaaagatatagaagaacaagtgacatggttcgaggaagtttcagaacaagaatttgacgatgatatttttggagcttgtagtacaaacactttttctttgagtgactattggggtaataaaggaaattggtgtactgctactcacgaatgtatgtcttggtgtaaataa

SEQ ID NO: 296 - protein, 67 aa, Staphylococcus aureus
MKNELGKFLEENELELGKFSESDMLEITDDEVYAAGTPLALLGGAATGVIGYISNQTCPTTACTRAC

SEQ ID NO: 297 - DNA, 204 bp, Staphylococcus aureus
atgaaaaatgaattaggtaagtttttagaagaaaacgaattagagttaggtaaattttcagaatcagacatgctagaaattactgatgatgaagtatatgcagctggaacacctttagccttattgggtggagctgccaccggggtgataggttatatttctaaccaaacatgtccaacaactgcttgtacacgcgcttgctag

SEQ ID NO: 298 - protein, 46 aa, Streptococcus pyogenes
MNNTIKDFDLDLKTNKKDTATPYVGSRYLCTPGSCWKLVCFTTTVK

SEQ ID NO: 299 - DNA, 141 bp, Streptococcus pyogenes
atgaataacacaattaaagactttgatctcgatttgaaaacaaataaaaaagacactgctacaccttatgttggtagccgttacctatgtacccctggttcttgttggaaattagtttgctttacaacaactgttaaataa

SEQ ID NO: 300 - protein, 51 aa, Streptococcus pyogenes
MEKNNEVINSIQEVSLEELDQIIGAGKNGVFKTISHECHLNTWAFLATCCS

SEQ ID NO: 301 - DNA, 156 bp, Streptococcus pyogenes
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgttcataa

SEQ ID NO: 302 - protein, 51 aa, Streptococcus pyogenes serotype M49
MTKEHEIINSIQEVSLEELDQIIGAGKNGVFKTISHECHLNTWAFLATCCS

SEQ ID NO: 303 - DNA, 156 bp, Streptococcus pyogenes serotype M49
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgctcataa

SEQ ID NO: 304 - protein, 56 aa, Bacillus subtilis
MEKLFKEVKLEELENQKGSGLGKAQCAALWLQCASGGTIGCGGGAVACQNYRQFCR

SEQ ID NO: 305 - DNA, 171 bp, Bacillus subtilis
atggaaaagctatttaaagaagttaaactagaggaactcgaaaaccaaaaaggtagtggattaggaaaagctcagtgtgctgcgttgtggctacaatgtgctagtggcggtacaattggttgtggtggcggagctgttgcttgtcaaaactatcgtcaattctgcagataa

SEQ ID NO: 306 - protein, 56 aa, Bacillus subtilis
MSKFDDFDLDVVKVSKQDSKITPQWKSESLCTPGCVTGALQTCFLQTLTCNCKISK

SEQ ID NO: 307 - DNA, 171 bp, Bacillus subtilis
atgtcaaagttcgatgatttcgatttggatgttgtgaaagtctctaaacaagactcaaaaatcactccgcaatggaaaagtgaatcactttgtacaccaggatgtgtaactggtgcattgcaaacttgcttccttcaaacactaacttgtaactgcaaaatctctaaataa

SEQ ID NO: 308 - protein, 50 aa, Bacillus subtilis
MKLPVQQVYSVYGGKDLPKGHSHSTMPFLSKLQFLTKIYLLDIHTQPFFI

SEQ ID NO: 309 - DNA, 153 bp, Bacillus subtilis
ttgaaattgccggtgcaacaggtctattcggtctatgggggtaaggatctcccaaaagggcatagtcattctactatgccctttttaagtaaattacaatttttaactaaaatctacctcttggatatacatacacaaccgtttttcatttga

SEQ ID NO: 310 - protein, 43 aa, Bacillus subtilis
MKKAVIVENKGCATCSIGAACLVDGPIPDFEIAGATGLFGLWG

SEQ ID NO: 311 - DNA, 132 bp, Bacillus subtilis
atgaaaaaagctgtcattgtagaaaacaaaggttgtgcaacatgctcgatcggagccgcttgtctagtggacggtcctatccctgattttgaaattgccggtgcaacaggtctattcggtctatgggggtaa

SEQ ID NO: 312 - protein, 58 aa, Streptococcus thermophilus; identical to SEQ ID NO: 24

SEQ ID NO: 313 - DNA, 177 bp, Streptococcus thermophilus
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcctgtaaaaattgtgcttaa

SEQ ID NO: 314 - protein, 64 aa, Streptococcus thermophilus
MKQYNGFEVLHELDLANVTGGQINWGSVVGHCIGGAIIGGAFSGGAAAGVGCLVGSGKAIINGL

SEQ ID NO: 315 - DNA, 195 bp, Streptococcus thermophilus
atgaagcagtataatggttttgaggttctacatgaacttgacttagcaaatgtaactggcggtcaaattaattggggatcagttgtaggacactgtataggtggagctattatcggaggtgcattttcaggaggtgcagcggctggagtaggatgccttgttgggagcggaaaggcaatcataaatggattataa

SEQ ID NO: 316 - protein, 85 aa, Streptococcus thermophilus
MNTITICKFDVLDAELLSTVEGGYSGKDCLKDMGGYALAGAGSGALWGAPAGGVGALPGAFVGAHVGAIAGGFACMGGMIGNKFN

SEQ ID NO: 317 - DNA, 258 bp, Streptococcus thermophilus
atgaatacaataactatttgtaaatttgatgttttagatgctgaacttctttcgacagttgagggtggatactctggtaaggattgtttaaaagacatgggaggatatgcattggcaggagctggaagtggagctctgtggggagctccagcaggaggtgttggagcacttccaggtgcatttgtcggagctcatgttggggcaattgcaggaggctttgcatgtatgggtggaatgattggtaataagtttaactaa

SEQ ID NO: 318 - protein, 52 aa, Bacillus cereus
MSEIKKALNTLEIEDFDAIEMVDVDAMPENEALEIMGASCTTCVCTCSCCTT

SEQ ID NO: 319 - DNA, 159 bp, Bacillus cereus
atgagtgaaattaaaaaagcattaaatacgcttgaaattgaagattttgatgcaattgaaatggttgatgttgatgctatgccagaaaacgaagcgcttgaaattatgggagcgtcatgtacgacatgcgtatgtacatgcagttgttgtacaacttga

SEQ ID NO: 320 - protein, 47 aa, Bacillus cereus
MEVMNNALITKVDEEIGGNAACVIGCIGSCVISEGIGSLVGTAFTLG

SEQ ID NO: 321 - DNA, 144 bp, Bacillus cereus
atggaagttatgaacaatgctttaattacaaaagtagatgaggagattggaggaaacgctgcttgtgtaattggttgtattggcagttgcgtaattagtgaaggaattggttcacttgtaggaacagcatttactttaggttaa

SEQ ID NO: 322 - protein, 49 aa, Bacillus cereus
MEVLNKQNVNIIPESEEVGGWVACVGACGTVCLASGGVGTEFAAASYFL

SEQ ID NO: 323 - DNA, 150 bp, Bacillus cereus
atggaagttttaaacaaacaaaatgtaaatattattccagaatctgaagaagtaggtggatgggtagcatgtgttggagcatgtggtacagtatgtcttgctagtggtggtgttggaacagagtttgcagctgcatcttatttcctataa

SEQ ID NO: 324 - protein, 40 aa, Bacillus thuringiensis
METPVVQPRDWTCWSCLVCAACSVELLNLVTAATGASTAS

SEQ ID NO: 325 - DNA, 123 bp, Bacillus thuringiensis
atggaaacaccagtagtacaaccaagggattggacttgttggagttgcttagtatgtgcagcatgttctgtggaattattaaatttagttactgcggcaacaggggctagtactgcaagctaa

SEQ ID NO: 326 - protein, 42 aa, Rhizobium leguminosarum bv. trifolii
MDNKVAKNVEVKKGSIKATFKAAVLKSKTKVDIGGSRQGCVA

SEQ ID NO: 327 - DNA, 129 bp, Rhizobium leguminosarum bv. trifolii
atggataacaaggttgcgaagaatgtcgaagtgaagaagggctccatcaaggcgaccttcaaggctgctgttctgaagtcgaagacgaaggtcgacatcggaggtagccgtcagggctgcgtcgcttaa

SEQ ID NO: 328 - protein, 70 aa, Streptococcus uberis
MNTIEKFENIKLFSLKKIIGGKTVNYGNGLYCNQKKCWVNWSETATTIVNNSIMNGLTGGNAGWHSGGRA

SEQ ID NO: 329 - DNA, 213 bp, Streptococcus uberis
atgaatacaattgaaaaatttgaaaatattaaacttttttcactaaagaaaattatcggtggcaaaactgtaaattatggtaatggcctttattgtaaccaaaaaaaatgctgggtaaactggtcagaaactgctacaacaatagtaaataattccatcatgaacgggctcacaggtggtaatgcgggttggcactcaggcgggagagcataa

SEQ ID NO: 330 - protein, 76 aa, Streptococcus uberis
MDILLELAGYTGIASGTAKKVVDAIDKGAAAFVIISIISTVISAGALGAVSASADFIILTVKNYISRNLKAQAVIW

SEQ ID NO: 331 - DNA, 231 bp, Streptococcus uberis
atggacattttattagaactcgcaggatatactgggatagcctcaggtactgcaaaaaaagttgttgatgccattgataaaggagctgcagcctttgttattatttcaattatctcaacagtaattagtgcgggagcattgggagcagtttcagcctcagctgattttattattttaactgtaaaaaattacattagtagaaatttaaaagcacaagctgtcatttggtaa

SEQ ID NO: 332 - protein, 64 aa, Clostridium perfringens
MDSELFKLMATQGAFAILFSYLLFYVLKENSKREDKYQNIIEELTELLPKIKEDVEDIKEKLNK

SEQ ID NO: 333 - DNA, 195 bp, Clostridium perfringens
atggatagtgaattatttaagttaatggcaacacaaggagcctttgcaatattattttcgtatttattgttttatgttttaaaagagaatagtaaaagagaagataagtatcaaaatataatagaggagcttacagaattattgccaaaaataaaagaagatgtagaagatataaaagaaaaacttaataaatag

SEQ ID NO: 334 - protein, 47 aa, Micrococcus varians
MTNAFQALDEVTDAELDAILGGGSGVIPTISHECHMNSFQFVFTCCS

SEQ ID NO: 335 - DNA, 144 bp, Micrococcus varians
atgacgaacgcatttcaggcactggacgaagtcacggacgccgagctcgacgccatccttggcgggggcagtggtgttattcccacgatcagccacgagtgccacatgaactccttccagttcgtgttcacctgctgctcctga

SEQ ID NO: 336 - protein, 285 aa, Streptococcus equi subsp. zooepidemicus
MKRIFFAFLSLCLFIFGTQTVSAATYTRPLDTGNITTGFNGYPGHVGVDYAVPVGTPVRAVANGTVKFAGNGANHPWMLWMAGNCVLIQHADGMHTGYAHLSKISVSTDSTVKQGQIIGYTGATGQVTGPHLHFEMLPANPNWQNGFSGRIDPTGYIANAPVFNGTTPTEPTTPTTNLKIYKVDDLQKINGIWQVRNNILVPTDFTWVDNGIAADDVIEVTSNGTRTSDQVLQKGGYFVINPNNVKSVGTPMKGSGGLSWAQVNFTTGGNVWLNTTSKDNLLYGK

SEQ ID NO: 337 - DNA, 858 bp, Streptococcus equi subsp. zooepidemicus
atgaaacgtatattttttgctttcttaagtttatgcttatttatattcggaacacaaacggtatctgcagctacttatactcggccattagatacgggaaatatcactacagggtttaacggataccctggtcatgttggagtcgattatgcagtacccgttggaactccggttagagcagttgcaaatggtacagtcaaatttgcaggtaatggggctaatcacccatggatgctttggatggctggaaactgtgttctaattcaacatgctgacgggatgcatactggatatgcacacttatcaaaaatttcagttagcacagatagtacagttaaacaaggacaaatcataggttatactggtgccaccggccaagttaccggtccacatttgcattttgaaatgttgccagcaaatcctaactggcaaaatggtttttctggaagaatagatccaaccggatacatcgctaatgcccctgtatttaatggaacaacacctacagaacctactactcctacaacaaatttaaaaatctataaagttgatgatttacaaaaaattaatggtatttggcaagtaagaaataacatacttgtaccaactgatttcacatgggttgataatggaattgcagcagatgatgtaattgaagtaactagcaatggaacaagaacctctgaccaagttcttcaaaaaggtggttattttgtcatcaatcctaataatgttaaaagtgttggaactccgatgaaaggtagtggtggtctatcttgggctcaagtaaactttacaacaggtggaaatgtctggttaaatactactagcaaagacaacttactttacggaaaataa

SEQ ID NO: 338 - protein, 45 aa, Myxococcus fulvus
ANCSCSTASDYCPILTFCTTGTACSYTPTGCGTGWVYCACNGNFY

SEQ ID NO: 339 - DNA, 135 bp, Artificial Sequence
  Polynucleotide encoding the polypeptide of SEQ ID NO 338
gcgaactgcagctgcagcaccgcgagcgattattgcccgattctgaccttttgcaccaccggcaccgcgtgcagctataccccgaccggctgcggcaccggctgggtgtattgcgcgtgcaacggcaacttttat

SEQ ID NO: 340 - protein, 19 aa, Streptomyces griseoluteus
CANSCSYGPLTWSCDGNTK

SEQ ID NO: 341 - DNA, 57 bp, Artificial Sequence
  Polynucleotide encoding the polypeptide of SEQ ID NO 340
tgcgcgaacagctgcagctatggcccgctgacctggagctgcgatggcaacaccaaa

SEQ ID NO: 342 - protein, 19 aa, Streptoverticillium griseoverticillatum
CKQSCSFGPFTFVCDGNTK

SEQ ID NO: 343 - DNA, 57 bp, Artificial Sequence
  Polynucleotide encoding the polypeptide of SEQ ID NO 342
tgcaaacagagctgcagctttggcccgtttacctttgtgtgcgatggcaacaccaaa

SEQ ID NO: 344 - protein, 7 aa, Carnobacterium sp.
GSEIQPR

SEQ ID NO: 345 - DNA, 21 bp, Artificial Sequence
  Polynucleotide encoding the polypeptide of SEQ ID NO 344
ggcagcgaaattcagccgcgc

SEQ ID NO: 346 - protein, 39 aa, Lactococcus lactis subsp. lactis
GTWDDIGQGIGRVAYWVGKAMGNMSDVNQASRINRKKKH

SEQ ID NO: 347 - DNA, 117 bp, Artificial Sequence
  Polynucleotide encoding the polypeptide of SEQ ID NO 346
ggcacctgggatgatattggccagggcattggccgcgtggcgtattgggtgggcaaagcgatgggcaacatgagcgatgtgaaccaggcgagccgcattaaccgcaaaaaaaaacat

SEQ ID NO: 348 - protein, 35 aa, Lactococcus lactis subsp. lactis
KKWGWLAWVDPAYEFIKGFGKGAIKEGNKDKWKNI

SEQ ID NO: 349 - DNA, 105 bp, Artificial Sequence
  Polynucleotide encoding the polypeptide of SEQ ID NO 348
aaaaaatggggctggctggcgtgggtggatccggcgtatgaatttattaaaggctttggcaaaggcgcgattaaagaaggcaacaaagataaatggaaaaacatt

SEQ ID NO: 350 - protein, 19 aa, Streptomyces sp.
CVQSCSFGPLTWSCDGNTK

SEQ ID NO: 351 - DNA, 57 bp, Artificial Sequence
  Polynucleotide encoding the polypeptide of SEQ ID NO 350
tgcgtgcagagctgcagctttggcccgctgacctggagctgcgatggcaacaccaaa

SEQ ID NO: 352 - protein, 19 aa, Actinoplanes liguriae
SSGWVCTLTIECGTVICAC

SEQ ID NO: 353 - DNA, 57 bp, Artificial Sequence
  Polynucleotide encoding the polypeptide of SEQ ID NO 352
agcagcggctgggtgtgcaccctgaccattgaatgcggcaccgtgatttgcgcgtgc

SEQ ID NO: 354 - protein, 38 aa, Lactobacillus curvatus
  VARIANT 34: Xaa = any amino acid
  VARIANT 37: Xaa = any amino acid
YTAKQCLQAIGSCGIAGTGAGAAGGPAGAFVGAXVVXI

SEQ ID NO: 355 - DNA, 114 bp, Artificial Sequence
  Polynucleotide encoding the polypeptide of SEQ ID NO 354
  misc_feature 100, 101, 102, 109, 110, 111: n = A,T,C or G
  misc_feature (100)...(102): nnn = a codon other than a stop codon
  misc_feature (109)...(111): nnn = a codon other than a stop codon
tataccgcgaaacagtgcctgcaggcgattggcagctgcggcattgcgggcaccggcgcgggcgcggcgggcggcccggcgggcgcgtttgtgggcgcgnnngtggtgnnnatt

SEQ ID NO: 356 - protein, 42 aa, Lactobacillus sakei
  VARIANT 32: Xaa = any amino acid
TKYYGNGVYCNSKKCWVDWGQAAGGIGQTVVXGWLGGAIPGK

SEQ ID NO: 357 - DNA, 126 bp, Artificial Sequence
  Polynucleotide encoding the polypeptide of SEQ ID NO 356
  misc_feature (94)...(96): nnn = any amino acid-coding triplet
accaaatattatggcaacggcgtgtattgcaacagcaaaaaatgctgggtggattggggccaggcggcgggcggcattggccagaccgtggtgnnnggctggctgggcggcgcgattccgggcaaa

SEQ ID NO: 358 - protein, 22 aa, Streptococcus mutans
FKSWSFCTPGCAKTGSFNSYCC

SEQ ID NO: 359 - DNA, 132 bp, Artificial Sequence
  Polynucleotide encoding the polypeptide of SEQ ID NO 358
tttaaaagctggagcttttgcaccccgggctgcgcgaaaaccggcagctttaacagctattgctgctttaaaagctggagcttttgcaccccgggctgcgcgaaaaccggcagctttaacagctattgctgc

SEQ ID NO: 360 - protein, 43 aa, Enterococcus mundtii
KYYGNGVSCNKKGCSVDWGKAIGIIGNNSAANLATGGAAGWSK

SEQ ID NO: 361 - DNA, 129 bp, Artificial Sequence
  Polynucleotide encoding the polypeptide of SEQ ID NO 360
aaatattatggcaacggcgtgagctgcaacaaaaaaggctgcagcgtggattggggcaaagcgattggcattattggcaacaacagcgcggcgaacctggcgaccggcggcgcggcgggctggagcaaa

SEQ ID NO: 362 - protein, 41 aa, Lactobacillus sakei
  VARIANT 9, 14, 33, 37: Xaa = any amino acid
KYYGNGVHXGKHSXTVDWGTAIGNIGNNAAANXATGXNAGG

SEQ ID NO: 363 - DNA, 123 bp, Artificial Sequence
  Polynucleotide encoding the polypeptide of SEQ ID NO 362
  misc_feature (25)...(27): nnn = any amino acid-coding triplet
  misc_feature (40)...(42): nnn = any amino acid-coding triplet
  misc_feature (97)...(99): nnn = any amino acid-coding triplet
  misc_feature (109)...(111): nnn = any amino acid-coding triplet
aaatattatggcaacggcgtgcatnnnggcaaacatagcnnnaccgtggattggggcaccgcgattggcaacattggcaacaacgcggcggcgaacnnngcgaccggcnnnaacgcgggcggc

SEQ ID NO: 364 - protein, 31 aa, Lactobacillus paracasei
GMSGYIQGIPDFLKGYLHGISAANKHKKGRL

SEQ ID NO: 365 - DNA, 93 bp, Artificial Sequence
  Polynucleotide encoding the polypeptide of SEQ ID NO 364
ggcatgagcggctatattcagggcattccggattttctgaaaggctatctgcatggcattagcgcggcgaacaaacataaaaaaggccgcctg

SEQ ID NO: 366 - protein, 31 aa, Leuconostoc mesenteroides
KGKGFWSWASKATSWLTGPQQPGSPLLKKHR

SEQ ID NO: 367 - DNA, 93 bp, Artificial Sequence
  Polynucleotide encoding the polypeptide of SEQ ID NO 366
aaaggcaaaggcttttggagctgggcgagcaaagcgaccagctggctgaccggcccgcagcagccgggcagcccgctgctgaaaaaacatcgc

SEQ ID NO: 368 - protein, 43 aa, Leuconostoc mesenteroides
KNYGNGVHCTKKGCSVDWGYAWTNIANNSVMNGLTGGNAGWHN

SEQ ID NO: 369 - DNA, 129 bp, Artificial Sequence
  Polynucleotide encoding the polypeptide of SEQ ID NO 368
aaaaactatggcaacggcgtgcattgcaccaaaaaaggctgcagcgtggattggggctatgcgtggaccaacattgcgaacaacagcgtgatgaacggcctgaccggcggcaacgcgggctggcataac

SEQ ID NO: 370 - protein, 47 aa, Bacillus subtilis
AIKLVQSPNGNFAASFVLDGTKWIFKSKYYDSSKGYWVGIYEVWDRK

SEQ ID NO: 371 - DNA, 141 bp, Artificial Sequence
  Polynucleotide encoding the polypeptide of SEQ ID NO 370
gcgattaaactggtgcagagcccgaacggcaactttgcggcgagctttgtgctggatggcaccaaatggatttttaaaagcaaatattatgatagcagcaaaggctattgggtgggcatttatgaagtgtgggatcgcaaa

SEQ ID NO: 372 - protein, 12 aa, Bacillus licheniformis
  VARIANT 7: Xaa = any amino acid
ISLEICXIFHDN

SEQ ID NO: 373 - DNA, 36 bp, Artificial Sequence
  Polynucleotide encoding the polypeptide of SEQ ID NO 372
  misc_feature (19)...(21): nnn = any amino acid-coding triplet
attagcctggaaatttgcnnnatttttcatgataac

SEQ ID NO: 374 - protein, 37 aa, Lactococcus lactis subsp. lactis
TSYGNGVHCNKSKCWIDVSELETYKAGTVSNPKDILW

SEQ ID NO: 375 - DNA, 111 bp, Artificial Sequence
  Polynucleotide encoding the polypeptide of SEQ ID NO 374
accagctatggcaacggcgtgcattgcaacaaaagcaaatgctggattgatgtgagcgaactggaaacctataaagcgggcaccgtgagcaacccgaaagatattctgtgg

SEQ ID NO: 376 - protein, 9 aa, Serratia plymuthica
DYHHGVRVL

SEQ ID NO: 377 - DNA, 27 bp, Artificial Sequence
  Polynucleotide encoding the polypeptide of SEQ ID NO 376
gattatcatcatggcgtgcgcgtgctg

SEQ ID NO: 378 - protein, 15 aa, Halobacterium sp.
DIDITGCSACKYAAG

SEQ ID NO: 379 - DNA, 45 bp, Artificial Sequence
  Polynucleotide encoding the polypeptide of SEQ ID NO 378
gatattgatattaccggctgcagcgcgtgcaaatatgcggcgggc

SEQ ID NO: 380 - protein, 12 aa, Bacillus subtilis
  VARIANT 1, 2, 6: Xaa = any amino acid
XXKEIXHIFHDN

SEQ ID NO: 381 - DNA, 36 bp, Artificial Sequence
  Polynucleotide encoding the polypeptide of SEQ ID NO 380
  misc_feature (1)...(3): nnn = any amino acid-coding triplet
  misc_feature (4)...(6): nnn = any amino acid-coding triplet
  misc_feature (16)...(18): nnn = any amino acid-coding triplet
nnnnnnaaagaaattnnncatatttttcatgataac

SEQ ID NO: 382 - protein, 12 aa, Lactobacillus curvatus
TPVVNPPFLQQT

SEQ ID NO: 383 - DNA, 36 bp, Artificial Sequence
  Polynucleotide encoding the polypeptide of SEQ ID NO 382
accccggtggtgaacccgccgtttctgcagcagacc

SEQ ID NO: 384 - protein, 10 aa, Lactobacillus curvatus
  VARIANT 10: Xaa = any amino acid
VAPFPEQFLX

SEQ ID NO: 385 - DNA, 30 bp, Artificial Sequence
  Polynucleotide encoding the polypeptide of SEQ ID NO 384
  misc_feature (28)...(30): nnn = any amino acid-coding triplet
gtggcgccgtttccggaacagtttctgnnn

SEQ ID NO: 386 - protein, 9 aa, Lactobacillus curvatus
NIPQLTPTP

SEQ ID NO: 387 - DNA, 27 bp, Artificial Sequence
  Polynucleotide encoding the polypeptide of SEQ ID NO 386
aacattccgcagctgaccccgaccccg

SEQ ID NO: 388 - protein, 18 aa, Bacillus thuringiensis subsp. entomocidus
  VARIANT 4, 7, 10: Xaa = any amino acid
DWTXWSXLVXAACSVELL

SEQ ID NO: 389 - DNA, 54 bp, Artificial Sequence
  Polynucleotide encoding the polypeptide of SEQ ID NO 388
  misc_feature (10)...(12): nnn = any amino acid-coding triplet
  misc_feature (19)...(21): nnn = any amino acid-coding triplet
  misc_feature (28)...(30): nnn = any amino acid-coding triplet
gattggaccnnntggagcnnnctggtgnnngcggcgtgcagcgtggaactgctg

SEQ ID NO: 390 - protein, 30 aa, Lactobacillus curvatus
AYPGNGVHCGKYSCTVDKQTAIGNIGNNAA

SEQ ID NO: 391 - DNA, 90 bp, Artificial Sequence
  Polynucleotide encoding the polypeptide of SEQ ID NO 390
gcgtatccgggcaacggcgtgcattgcggcaaatatagctgcaccgtggataaacagaccgcgattggcaacattggcaacaacgcggcg

SEQ ID NO: 392 - protein, 43 aa, Carnobacterium divergens
TKYYGNGVYCNSKKCWVDWGTAQGCIDVVIGQLGGGIPGKGKC

SEQ ID NO: 393 - DNA, 129 bp, Artificial Sequence
  Polynucleotide encoding the polypeptide of SEQ ID NO 392
accaaatattatggcaacggcgtgtattgcaacagcaaaaaatgctgggtggattggggcaccgcgcagggctgcattgatgtggtgattggccagctgggcggcggcattccgggcaaaggcaaatgc

SEQ ID NO: 394 - protein, 62 aa, Enterococcus sp.
NRWYCNSAAGGVGGAAVCGLAGYVGEAKENIAGEVRKGWGMAGGFTHNKACKSFPGSGWASG

SEQ ID NO: 395 - DNA, 186 bp, Artificial Sequence
  Polynucleotide encoding the polypeptide of SEQ ID NO 394
aaccgctggtattgcaacagcgcggcgggcggcgtgggcggcgcggcggtgtgcggcctggcgggctatgtgggcgaagcgaaagaaaacattgcgggcgaagtgcgcaaaggctggggcatggcgggcggctttacccataacaaagcgtgcaaaagctttccgggcagcggctgggcgagcggc

SEQ ID NO: 396 - protein, 39 aa, Enterococcus faecium
TTKNYGNGVCNSVNWCQCGNVWASCNLATGCAAWLCKLA

SEQ ID NO: 397 - DNA, 117 bp, Artificial Sequence
  Polynucleotide encoding the polypeptide of SEQ ID NO 396
accaccaaaaactatggcaacggcgtgtgcaacagcgtgaactggtgccagtgcggcaacgtgtgggcgagctgcaacctggcgaccggctgcgcggcgtggctgtgcaaactggcg

SEQ ID NO: 398 - protein, 30 aa, Paenibacillus polymyxa
ASIIKTTIKVSKAVCKTLTCICTGSCSNCK

SEQ ID NO: 399 - DNA, 90 bp, Artificial Sequence
  Polynucleotide encoding the polypeptide of SEQ ID NO 398
gcgagcattattaaaaccaccattaaagtgagcaaagcggtgtgcaaaaccctgacctgcatttgcaccggcagctgcagcaactgcaaa

SEQ ID NO: 400 - protein, 31 aa, Staphylococcus epidermidis
SASIVKTTIKASKKLCRGFTLTCGCHFTGKK

SEQ ID NO: 401 - DNA, 93 bp, Artificial Sequence
  Polynucleotide encoding the polypeptide of SEQ ID NO 400
agcgcgagcattgtgaaaaccaccattaaagcgagcaaaaaactgtgccgcggctttaccctgacctgcggctgccattttaccggcaaaaaa

SEQ ID NO: 402 - protein, 43 aa, Enterococcus faecium
KYYGNGVSCNKKGCSVDWGKAIGIIGNNAAANLTTGGKAAWAC

SEQ ID NO: 403 - DNA, 129 bp, Artificial Sequence
  Polynucleotide encoding the polypeptide of SEQ ID NO 402
aaatattatggcaacggcgtgagctgcaacaaaaaaggctgcagcgtggattggggcaaagcgattggcattattggcaacaacgcggcggcgaacctgaccaccggcggcaaagcggcgtgggcgtgc

SEQ ID NO: 404 - protein, 39 aa, Paenibacillus polymyxa
ATYYGNGLYCNKQKHYTWVDWNKASREIGKITVNGWVQH

SEQ ID NO: 405 - DNA, 117 bp, Artificial Sequence
  Polynucleotide encoding the polypeptide of SEQ ID NO 404
gcgacctattatggcaacggcctgtattgcaacaaacagaaacattatacctgggtggattggaacaaagcgagccgcgaaattggcaaaattaccgtgaacggctgggtgcagcat

SEQ ID NO: 406 - protein, 35 aa, Bacillus circulans
VNYGNGVSCSKTKCSVNWGIITHQAFRVTSGVASG

SEQ ID NO: 407 - DNA, 105 bp, Artificial Sequence
  Polynucleotide encoding the polypeptide of SEQ ID NO 406
gtgaactatggcaacggcgtgagctgcagcaaaaccaaatgcagcgtgaactggggcattattacccatcaggcgtttcgcgtgaccagcggcgtggcgagcggc

SEQ ID NO: 408 - protein, 30 aa, Paenibacillus polymyxa
FVYGNGVTSILVQAQFLVNGQRRFFYTPDK

SEQ ID NO: 409 - DNA, 90 bp, Artificial Sequence
  Polynucleotide encoding the polypeptide of SEQ ID NO 408
tttgtgtatggcaacggcgtgaccagcattctggtgcaggcgcagtttctggtgaacggccagcgccgctttttttataccccggataaa

SEQ ID NO: 410 - protein, 13 aa, Lactobacillus rhamnosus
AVPAVRKTNETLD

SEQ ID NO: 411 - DNA, 39 bp, Artificial Sequence
  Polynucleotide encoding the polypeptide of SEQ ID NO 410
gcggtgccggcggtgcgcaaaaccaacgaaaccctggat

SEQ ID NO: 412 - protein, 69 aa, Bacillus licheniformis
MKNSAAREAFKGANHPAGMVSEEELKALVGGNDVNPETTPATTSSWTCITAGVTVSASLCPTTKCTSRC

SEQ ID NO: 413 - DNA, 207 bp, Artificial Sequence
  Polynucleotide encoding the polypeptide of SEQ ID NO 412
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgcccgaccaccaaatgcaccagccgctgc

SEQ ID NO: 414 - protein, 36 aa, Lactobacillus plantarum
KYYGNGLSCSKKGCTVNWGQAFSCGVNRVATAGHGK

SEQ ID NO: 415 - DNA, 108 bp, Artificial Sequence
  Polynucleotide encoding the polypeptide of SEQ ID NO 414
aaatattatggcaacggcctgagctgcagcaaaaaaggctgcaccgtgaactggggccaggcgtttagctgcggcgtgaaccgcgtggcgaccgcgggccatggcaaa

SEQ ID NO: 416 - protein, 24 aa, Lactobacillus acidophilus
GNPKVAHCASQIGRSTAWGAVSGA

SEQ ID NO: 417 - DNA, 72 bp, Artificial Sequence
  Polynucleotide encoding the polypeptide of SEQ ID NO 416
ggcaacccgaaagtggcgcattgcgcgagccagattggccgcagcaccgcgtggggcgcggtgagcggcgcg

SEQ ID NO: 418 - protein, 40 aa, Enterococcus faecalis; identical to SEQ ID NO: 1

SEQ ID NO: 419 - DNA, 120 bp, Artificial Sequence
  Polynucleotide encoding the polypeptide of SEQ ID NO 418
tggctgccgccggcgggcctgctgggccgctgcggccgctggtttcgcccgtggctgctgtggctgcagagcggcgcgcagtataaatggctgggcaacctgtttggcctgggcccgaaa

SEQ ID NO: 420 - protein, 40 aa, Anabaena variabilis
NLDQWLTEQVHEFQDMYLEPQAISNQDITFKLSDLDFIHN

SEQ ID NO: 421 - DNA, 124 bp, Anabaena variabilis
taatttagatcagtggttaacagaacaagttcatgagtttcaagatatgtacttggaaccacaagcaatatccaatcaagacattaccttcaaactatctgacctagattttattcataattga

SEQ ID NO: 422 - protein, 40 aa, Nostoc sp; identical to SEQ ID NO: 420

SEQ ID NO: 423 - DNA, 123 bp, Nostoc sp
aatttagatcaatggttaacagaacaagttcatgagtttcaagatatgtacttggaaccacaagcaatatccaatcaagacattaccttcaaactgtcagacctagattttattcataattga

SEQ ID NO: 424 - protein, 7 aa, Nostoc azollae
HREKKSA

SEQ ID NO: 425 - DNA, 24 bp, Nostoc azollae
cacagagagaaaaaatcagcatag

SEQ ID NO: 426 - protein, 23 aa, Acaryochloris marina
TSNNWLAKNYLSMWNKKSSNPNL

SEQ ID NO: 427 - DNA, 72 bp, Acaryochloris marina
acaagcaataactggctagccaaaaactatctttctatgtggaataaaaagagcagtaatccaaacctttag

SEQ ID NO: 428 - protein, 6 aa, Cyanothece
FRYFWW

SEQ ID NO: 429 - DNA, 21 bp, Cyanothece
tttagatatttttggtggtaa

SEQ ID NO: 430 - protein, 6 aa, Cyanothece
FRYFWW

SEQ ID NO: 431 - DNA, 21 bp, Cyanothece
tttagatatttttggtggtaa

SEQ ID NO: 432 - protein, 9 aa, Cyanothece
CGEKWRIFS

SEQ ID NO: 433 - DNA, 27 bp, Cyanothece
tgtggagaaaaatggagaatttttagc

SEQ ID NO: 434 - protein, 8 aa, Cyanothece
FRLQLWQF

SEQ ID NO: 435 - DNA, 24 bp, Cyanothece
tttcgcttacaactgtggcaattt

SEQ ID NO: 436 - protein, 16 aa, Cyanothece
LGCNQSSIWSIFFWNH

SEQ ID NO: 437 - DNA, 51 bp, Cyanothece
ctaggatgtaaccagagcagtatctggtcaatttttttctggaatcattaa

SEQ ID NO: 438 - protein, 40 aa, Microcoleus chthonoplastes
YNLQGLPAIESEDCIPDSVAPSDDWFSGVSSLFNRLTGLG

SEQ ID NO: 439 - DNA, 123 bp, Microcoleus chthonoplastes
tataacctacaggggttgccagcaattgagtcagaagactgtatcccagattctgtagcgccttcggatgattggttttcaggcgtatcgtctctgtttaaccgcttgactgggttgggttag

SEQ ID NO: 440 - protein, 37 aa, Nostoc sp
WMAIRRILRCHPFHPGGYDPVPELGEHCCHHDSGNKG

SEQ ID NO: 441 - DNA, 114 bp, Nostoc sp
tggatggcgattcgccgcattttgcgttgtcatccattccacccagggggttatgatcctgtaccagagttgggtgagcattgttgtcatcatgatagcgggaataaggggtga

SEQ ID NO: 442 - protein, 35 aa, Anabaena variabilis
WMGIRRILRCHPFHPGGYDPVPEVGEHCCHHDSGK

SEQ ID NO: 443 - DNA, 108 bp, Anabaena variabilis
tggatggggattcgccgcattttgcgttgtcatccattccacccaggcggttatgatcctgtaccagaggtgggtgagcattgttgtcatcatgatagcgggaagtag

SEQ ID NO: 444 - protein, 48 aa, Nodularia spumigena
WMATRRILRCHPFHPGGYDPVPEVKHNCCDQHLSDSGKQTTEDHHKGS

SEQ ID NO: 445 - DNA, 147 bp, Nodularia spumigena
tggatggcgactcggcggattttgcgttgtcatcccttccatcctggtggatatgatccagttccagaggtaaaacacaattgctgcgatcagcatctgtccgattctgggaaacagaccacagaagaccatcacaaaggctcgtag

SEQ ID NO: 446 - protein, 34 aa, Nostoc azollae
WMATLRILRCHPFHPGGYDPVPGLAEKSCCDHHD

SEQ ID NO: 447 - DNA, 105 bp, Nostoc azollae
tggatggcaactttgcggattttacgctgtcatcctttccatcctggtggttatgatcctgtaccaggactagcggaaaaatcctgttgtgaccatcatgattga

SEQ ID NO: 448 - protein, 28 aa, Synechococcus
WLTAKRFCRCHPLHPGGYDPVPEKKSVL

SEQ ID NO: 449 - DNA, 87 bp, Synechococcus
tggctaacagccaagcgcttttgtcgctgtcatccgcttcatcctggcgggtatgatccggtaccggagaagaaatcggtactctaa

SEQ ID NO: 450 - protein, 23 aa, Prochlorococcus marinus
WLTLRRLSRCHPFTPCGCDPVPD

SEQ ID NO: 451 - DNA, 72 bp, Prochlorococcus marinus
tggctcaccctgcggcgcctgtctcgttgccatccttttaccccctgtggttgcgacccggtgcctgattaa

SEQ ID NO: 452 - protein, 69 aa, Escherichia coli
MSYKKLYQLTAIFSLPLTILLVSLSSLRIVGEGNSYVDVFLSFIIFLGFIELIHGIRKILVWSGWKNGS

SEQ ID NO: 453 - DNA, 210 bp, Escherichia coli
atgagttataaaaaactgtaccaattgacggctatatttagtttacctcttactatcttattggtttcactttcatcccttcggattgttggcgaagggaattcttatgttgacgtttttctaagctttataatatttcttggttttattgagctgattcatgggattcgaaagattttggtctggtcaggctggaaaaacggaagttaa

SEQ ID NO: 454 - protein, 85 aa, Escherichia coli
MGLKLDLTWFDKSTEDFKGEEYSKDFGDDGSVMESLGVPFKDNVNNGCFDVIAEWVPLLQPYFNHQIDISDNEYFVSFDYRDGDW

SEQ ID NO: 455 - DNA, 258 bp, Escherichia coli
atgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattggtga

SEQ ID NO: 456 - protein, 113 aa, Escherichia coli
MSLRYYIKNILFGLYCTLIYIYLITKNSEGYYFLVSDKMLYAIVISTILCPYSKYAIEYIAFNFIKKDFFERRKNLNNAPVAKLNLFMLYNLLCLVLAIPFGLLGLFISIKNN

SEQ ID NO: 457 - DNA, 342 bp, Escherichia coli
atgagcttaagatactacataaaaaatattttatttggcctgtactgcacacttatatatatataccttataacaaaaaacagcgaagggtattatttccttgtgtcagataagatgctatatgcaatagtgataagcactattctatgtccatattcaaaatatgctattgaatacatagcttttaacttcataaagaaagattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 458 - protein, 85 aa, Escherichia coli
MGLKLHIHWFDKKTEEFKGGEYSKDFGDDGSVIESLGMPLKDNINNGWFDVEKPWVSILQPHFKNVIDISKFDYFVSFVYRDGNW

SEQ ID NO: 459 - DNA, 258 bp, Escherichia coli
atggggcttaaattacatattcattggtttgataagaaaaccgaagagtttaaaggcggtgaatactcaaaagacttcggtgatgatggttctgtcattgaaagtctggggatgcctttaaaggataatattaataatggttggtttgatgttgaaaaaccatgggtttcgatattacagccacactttaaaaatgtaatcgatattagtaaatttgattactttgtatcctttgtttaccgggatggtaactggtaa

SEQ ID NO: 460 - protein, 86 aa, Escherichia coli
MELKHSISDYTEAEFLEFVKKICRAEGATEEDDNKLVREFERLTEHPDGSDLIYYPRDDREDSPEGIVKEIKEWRAANGKSGFKQG

SEQ ID NO: 461 - DNA, 261 bp, Escherichia coli
atggaactgaaacatagtattagtgattataccgaggctgaatttctggagtttgtaaaaaaaatatgtagagctgaaggtgctactgaagaggatgacaataaattagtgagagagtttgagcgattaactgagcacccagatggttcagatctgatttattatcctcgcgatgacagggaagatagtcctgaagggattgtcaaggaaattaaagaatggcgagctgctaacggtaagtcaggatttaaacagggctga

SEQ ID NO: 462 - protein, 178 aa, Citrobacter freundii
MMNEHSIDTDNRKANNALYLFIIIGLIPLLCIFVVYYKTPDALLLRKIATSTENLPSITSSYNPLMTKVMDIYCKTAPFLALILYILTFKIRKLINNTDRNTVLRSCLLSPLVYAAIVYLFCFRNFELTTAGRPVRLMATNDATLLLFYIGLYSIIFFTTYITLFTPVTAFKLLKKRQ

SEQ ID NO: 463 - DNA, 537 bp, Citrobacter freundii
atgatgaatgaacactcaatagatacggacaacagaaaggccaataacgcattgtatttatttataataatcggattaataccattattgtgcatttttgttgtttactacaaaacgccagacgctttacttttacgtaaaattgctacaagcactgagaatctcccgtcaataacatcctcctacaacccattaatgacaaaggttatggatatttattgtaaaacagcgcctttccttgccttaatactatacatcctaacctttaaaatcagaaaattaatcaacaacaccgacaggaacactgtacttagatcttgtttattaagtccattggtctatgcagcaattgtttatctattctgcttccgaaattttgagttaacaacagccggaaggcctgtcagattaatggccaccaatgacgcaacactattgttattttatattggtctgtactcaataattttctttacaacctatatcacgctattcacaccagtcactgcatttaaattattaaaaaaaaggcagtaa

SEQ ID NO: 464 - protein, 111 aa, Escherichia coli
MNRKYYFNNMWWGWVTGGYMLYMSWDYEFKYRLLFWCISLCGMVLYPVAKWYIEDTALKFTRPDFWNSGFFADTPGKMGLLAVYTGTVFILSLPLSMIYILSVIIKRLSVR

SEQ ID NO: 465 - DNA, 336 bp, Escherichia coli
atgaacagaaaatattattttaataatatgtggtggggatgggtgacggggggatatatgctgtatatgtcatgggattatgagtttaaatacagattactgttctggtgtatttctctctgcggaatggttttgtatccggttgcaaaatggtatattgaagatacagctctaaaatttacccggcctgatttctggaacagcggtttttttgctgatacacctggaaaaatggggttgcttgcggtttatacgggtactgttttcatattatctcttccgttaagtatgatatatattctttctgttattataaaaaggctgtctgtaagatag

SEQ ID NO: 466 - protein, 115 aa, Escherichia coli
MKLDISVKYLLKSLIPILIILTVFYLGWKDNQENARMFYAFIGCIISAITFPFSMRIIQKMVIRFTGKEFWQKDFFTNPVGGSLTAIFELFCFVISVPVVAIYLIFILCKALSGK

SEQ ID NO: 467 - DNA, 348 bp, Escherichia coli
atgaaactggatatatctgtaaagtatttactgaaaagcctgataccaatcctcattattcttacagttttttatctgggatggaaagataaccaggaaaatgcaagaatgttttatgcgttcatcggatgcattatcagtgccattacttttcctttttcaatgaggataatacagaaaatggtaataaggtttacagggaaagaattctggcaaaaagacttctttacaaatccagttggcggaagcttaactgcaatatttgaattattctgtttcgttatatcagttcctgtggttgccatttacttaatttttatactctgcaaagccctttcaggaaaatga

SEQ ID NO: 468 - protein, 131 aa, Escherichia coli
MHNTLLEKIIAYLSLPGFHSLNNPPLSEAFNLYVHTAPLAATSLFIFTHKELELKPKSSPLRALKILTPFTILYISMIYCFLLTDTELTLSSKTFVLIVKKRSVFVFFLYNTIYWDIYIHIFVLLVPYRNI

SEQ ID NO: 469 - DNA, 396 bp, Escherichia coli
atgcacaatacactcctcgaaaaaatcatcgcatacctatccctaccaggatttcattcattaaacaacccgcccctaagcgaagcattcaatctctatgttcatacagcccctttagctgcaaccagcttattcatattcacacacaaagaattagagttaaaaccaaagtcgtcacctctgcgggcactaaagatattaactcctttcactattctttatatatccatgatatactgtttcttgctaactgacacagaactaaccttgtcatcaaaaacatttgtattaatagtcaaaaaacgatctgtttttgtcttttttctatataacactatatattgggatatatatattcacatatttgtacttttggttccttataggaacatataa

SEQ ID NO: 470 - protein, 85 aa, Escherichia coli
MELKNSISDYTETEFKKIIEDIINCEGDEKKQDDNLEHFISVTEHPSGSDLIYYPEGNNDGSPEAVIKEIKEWRAANGKSGFKQG

SEQ ID NO: 471 - DNA, 258 bp, Escherichia coli
atggaactgaaaaacagcattagtgattacactgaaactgaattcaaaaaaattattgaagacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 472 - protein, 98 aa, Lactococcus lactis subsp. lactis
MKKKQIEFENELRSMLATALEKDISQEERNALNIAEKALDNSEYLPKIILNLRKALTPLAINRTLNHDLSELYKFITSSKASNKNLGGGLIMSWGRLF

SEQ ID NO: 473 - DNA, 297 bp, Lactococcus lactis subsp. lactis
atgaaaaaaaaacaaatagaatttgaaaacgagctaagaagtatgttggctaccgcccttgaaaaagacattagtcaagaggaaagaaatgctctgaatattgcagaaaaggcgcttgacaattctgaatatttaccaaaaattattttaaacctcagaaaagccctaactccattagctataaatcgaacacttaaccatgatttatctgaactgtataaattcattacaagttccaaagcatcaaacaaaaatttaggtggtggtttaattatgtcgtggggacgactattctaa

SEQ ID NO: 474 - protein, 98 aa, Lactococcus lactis subsp. cremoris; identical to SEQ ID NO: 472

SEQ ID NO: 475 - DNA, 297 bp, Lactococcus lactis subsp. cremoris; identical to SEQ ID NO: 473

SEQ ID NO: 476 - protein, 87 aa, Escherichia coli
MNKMAMIDLAKLFLASKITAIEFSERICVERRRLYGVKDLSPNILNCGEELFMAAERFEPDADRANYEIDDNGLKVEVRSILEKFKL

SEQ ID NO: 477 - DNA, 249 bp, Escherichia coli
atgatcgatttggcgaaattatttttagcttcgaaaattacagtgattgagttttcagagcgaatttgtgttgaacggagaagattgtatggtgttaaggatttgtctccgaatatattaaattgtggggaagagttgtctatggctgctgagcgatttgagcctgatgcagatagggctaattatgaaattgatgataatggacttaaggtcgaggtccgatctatcttggaaaaacttaaatcataa

SEQ ID NO: 478 - protein, 83 aa, Escherichia coli
MKLSPKAAIEVCNEAAKKGLWILGIDGGHWLNPGFRIDSSASWTYDMPEEYKSKIPENNRLAIENIKDDIENGYTAFIITLKM

SEQ ID NO: 479 - DNA, 243 bp, Escherichia coli
atgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggagaatacaaatcaaaaatccctgaaaataatagattggctattgaaaatattaaagatgatattgagaatggatacactgctttcattatcacgttaa

SEQ ID NO: 480 - protein, 85 aa, Escherichia coli
MGLKLHINWFDKRTEEFKGGEYSKDFGDDGSVIERLGMPFKDNINNGWFDVIAEWVPLLQPYFNHQIDISDNEYFVSFDYRDGDW

SEQ ID NO: 481 - DNA, 258 bp, Escherichia coli
atggggcttaaattacatattaattggtttgataagacgaccgaggaatttaaaggtggtgagtattcaaaagattttggagatgatggctcggtcattgaacgtcttggaatgcctttaaaagataatatcaataatggttggtttgatgttatagctgaatgggtacctttgctacaaccatactttaatcatcaaattgatatttccgataatgagtattttgtttcgtttgattatcgtgatggtgattggtga

SEQ ID NO: 482 - protein, 85 aa, Escherichia coli
MELKKSIGDYTETEFKKIIENIINCEGDEKKQDDNLEHFISVTEHPSGSDLIYYPEGNNDGSPEAVIKEIKEWRAANGKSGFKQG

SEQ ID NO: 483 - DNA, 258 bp, Escherichia coli
gtggagctaaagaaaagtattggtgattacactgaaaccgaattcaaaaaaattattgaaaacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 484 - protein, 86 aa, Escherichia coli [sequence omitted]

SEQ ID NO: 485 - DNA, 261 bp, Escherichia coli
atggaactgaagcatagcattagtgattatacagaagctgaatttttacaacttgtaacaacaatttgtaatgcgaacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggctaa

SEQ ID NO: 486 - protein, 141 aa, Escherichia coli
MLTLYGYIRNVFLYRMNDRSCGDFMKVISMKFIFILTIIALAAVFFWSEDKGPACYQVSDEQARTFVKNDYLQRMKRWDNDVQLLGTEIPKITWEKIERSLTDVEDEKTLLVPFKAEGPDGKRMYYGMYHCEEGYVEYAND

SEQ ID NO: 487 - DNA, 354 bp, Escherichia coli
atgaaagtaattagcatgaaatttatttttattttaacgattattgctcttgctgctgtttttttctggtctgaagataaaggtccggcatgctatcaggtcagcgatgaacaggccagaacgtttgtaaaaaatgattacctgcaaagaatgaaacgctgggacaacgatgtacaacttcttggtacagaaatcccgaaaattacatgggaaaagattgagagaagtttaacagatgttgaagatgaaaaaacacttcttgtcccatttaaagctgaaggcccggacggtaagagaatgtattatggcatgtaccattgtgaggagggatatgttgaatatgcgaatgactaa

SEQ ID NO: 488 - protein, 175 aa, Escherichia coli
MTSNKDKNKKANEILYAFSIIGIIPLMAILILRINDPYSQVLYYLYNKVAFLPSITSLHDPVMTTLMSNYNKTAPVMGILVFLCTYKTREIIKPVTRKLVVQSCFWGPVFYAILIYITLFYNLELTTAGGFFKLLSHNVITLFILYCSIYFTVLTMTYAILLMPLLVIKYFKGRQ

SEQ ID NO: 489 - DNA, 528 bp, Escherichia coli
atgaccagcaataaagataagaacaagaaagcaaacgaaatattatatgcattttccataatcgggattattccattaatggctatattaatacttcgaataaatgatccatattctcaagtgctgtactacttatataataaggtggcatttctcccttctattacatcattgcatgatcccgtcatgacaacacttatgtcaaactacaacaagacagcgccagttatgggtattctcgtttttctttgcacatataagacaagagaaatcataaagccagtaacaagaaaacttgttgtgcaatcctgtttctgggggcccgttttttatgccattctgatttatatcacactgttctataatctggaactaacaacagcaggtggtttttttaaattattatctcataatgtcatcactctgtttattttatattgctccatttactttactgttttaaccatgacatatgcgattttactgatgccattacttgtcattaaatattttaaagggaggcagtaa

SEQ ID NO: 490 - protein, 78 aa, Escherichia coli
MDRKRTKLELLFAFIINATAIYIALAIYDCVFRGKDFLSMHTFCFSALMSAICYFVGDNYYSISDKIKRRSYENSDSK

SEQ ID NO: 491 - DNA, 237 bp, Escherichia coli
atggatagaaaaagaacaaaattagagttgttatttgcatttataataaatgccaccgcaatatatattgcattagctatatatgattgtgtttttagaggaaaggactttttatccatgcatacattttgcttctctgcattaatgtctgcaatatgttactttgttggtgataattattattcaatatccgataagataaaaaggagatcatatgagaactctgactctaaatga

SEQ ID NO: 492 - protein, 113 aa, Shigella sonnei
MSLRYYIKNILFGLYCALIYIYLITKNNEGYYFLASDKMLYAIVISTILCPYSKYAIEHIFFKFIKKDFFRKRKNLNKCPRGKIKPYLCVYNLLCLVLAIPFGLLGLVYINKE

SEQ ID NO: 493 - DNA, 342 bp, Shigella sonnei
atgagtttaagatactacataaaaaatattttgtttggcctatactgcgcacttatatatatataccttataacaaaaaacaacgaagggtattatttcctagcgtcagataagatgctatacgcaatagtgataagcactattctatgcccatattcaaaatatgctattgaacacatattttttaagttcataaagaaagattttttcagaaaaagaaaaaacctaaataaatgcccccgtggcaaaattaaaccgtatttatgcgtatacaatctactttgtttggtcctagcaatcccatttggattgctaggacttgtttatatcaataaagaataa

SEQ ID NO: 494 - protein, 113 aa, Escherichia coli
MSLRYYIKNILFGLYCTLIYIYLITKNSEEYYFLVTDKMLYAIVISTILCPYSKYAIEHIAFNFIKKHFFERRKNLNNAPVAKLNLFMLYNLLCLVLAIPFGLLGLFISIKNN

SEQ ID NO: 495 - DNA, 342 bp, Escherichia coli
atgagcttaagatactacataaaaaatattttatttggcctgtactgcacacttatatatatataccttataacaaaaaacagcgaagagtattatttccttgtgacagataagatgctatatgcaatagtgataagcactattctatgtccatattcaaaatatgctattgaacacatagcttttaacttcataaagaaacattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 496 - protein, 113 aa, Leuconostoc gelidum
MRKNNILLDDAKIYTNKLYLLLIDRKDDAGYGDICDVLFQVSKKLDSTKNVEALINRLVNYIRITASTNRIKFSKDEEAVIIELGVIGQKAGLNGQYMADFSDKSQFYSIFER

SEQ ID NO: 497 - DNA, 342 bp, Leuconostoc gelidum
ttgagaaaaaataacattttattggacgatgctaaaatatacacgaacaaactctatttgctattaatcgatagaaaagatgacgctgggtatggagatatttgtgatgttttgtttcaggtatccaaaaaattagatagcacaaaaaatgtagaagcattgattaaccgattggtcaattatatacgaattaccgcttcaacaaacagaattaagttttcaaaagatgaagaggctgtaattatagaacttggtgtaattggtcagaaggctggattaaacggccaatacatggctgatttttctgacaaatctcagttttatagtatctttgaaagataa

SEQ ID NO: 498 - protein, 91 aa, Lactococcus lactis subsp. cremoris
MKKKVDTEKQITSWASDLASKNETKVQEKLILSSYIQDIENHVYFPKAMISLEKKLRDQNNICALSKEVNQFYFKVVEVNQRKSWMVGLIV

SEQ ID NO: 499 - DNA, 276 bp, Lactococcus lactis subsp. cremoris
atgaaaaaaaaagttgatacagaaaaacaaattacttcttgggcatctgacttagcttccaaaaatgaaacaaaggttcaagaaaaattaatactgtcttcttatattcaggacatcgaaaaccatgtttactttccaaaagcaatgatttctttagaaaaaaaattacgagaccaaaataatatttgcgctttatcaaaagaagtcaatcagttttattttaaagttgttgaagtaaatcaaagaaaatcctggatggtaggtttgatagtttaa

SEQ ID NO: 500 - protein, 112 aa, Pediococcus acidilactici
MNKTKSEHIKQQALDLFTRLQFLLQKHDTIEPYQYVLDILETGISKTKHNQQTPERQARVVYNKIASQALVDKLHFTAEENKVLAAINELAHSQKGWGEFNMLDTTNTWPSQ

SEQ ID NO: 501 - DNA, 339 bp, Pediococcus acidilactici
atgaataagactaagtcggaacatattaaacaacaagctttggacttatttactaggctacagtttttactacagaagcacgatactatcgaaccttaccagtacgttttagatattctggagactggtatcagtaaaactaaacataaccagcaaacgcctgaacgacaagctcgtgtagtctacaacaagattgccagccaagcgttagtagataagttacattttactgccgaagaaaacaaagttctagcagccatcaatgaattggcgcattctcaaaaagggtggggcgagtttaacatgctagatactaccaatacgtggcctagccaatag

SEQ ID NO: 502 - protein, 88 aa, Carnobacterium maltaromaticum
MIKDEKINKIYALVKSALDNTDVKNDKKLSLLLMRIQETSINGELFYDYKKELQPAISMYSIQHNFRVPDDLVKLLALVQTPKAWSGF

SEQ ID NO: 503 - DNA, 267 bp, Carnobacterium maltaromaticum
atgataaaagatgaaaaaataaataaaatctatgctttagttaagagcgcacttgataatacggatgttaagaatgataaaaaactttctttacttcttatgagaatacaagaaacatcaattaatggagaactattttacgattataaaaaagaattacagccagctattagtatgtactctattcaacataactttcgggttcctgacgatctagtaaaactgttagcattagttcaaacacctaaagcttggtcagggttttaa

SEQ ID NO: 504 - protein, 111 aa, Carnobacterium maltaromaticum
MDIKSQTLYLNLSEAYKDPEVKANEFLSKLVVQCAGKLTASNSENSYIEVISLLSRGISSYYLSHKRIIPSSMLTIYTQIQKDIKNGNIDTEKLRKYEIAKGLMSVPYIYF

SEQ ID NO: 505 - DNA, 336 bp, Carnobacterium maltaromaticum
atggatataaagtctcaaacattatatttgaatctaagcgaggcatataaagaccctgaagtaaaagctaatgaattcttatcaaaattagttgtacaatgtgctgggaaattaacagcttcaaacagtgagaacagttatattgaagtaatatcattgctatctaggggtatttctagttattatttatcccataaacgtataattccttcaagtatgttaactatatatactcaaatacaaaaggatataaaaaacgggaatattgacaccgaaaaattaaggaaatatgagatagcaaaaggattaatgtccgttccttatatatatttctaa

SEQ ID NO: 506 - protein, 245 aa, Lactococcus lactis subsp. lactis
MRRYLILIVALIGITGLSGCYQTSHKKVRFDEGSYTNFIYDNKSYFVTDKEIPQENVNNSKVKFYKLLIVDMKSEKLLSSSNKNSVTLVLNNIYEASDKSLCMGINDRYYKILPESDKGAVKALRLQNFDVTSDISDDNFVIDKNDSRKIDYMGNIYSISDTTVSDEELGEYQDVLAEVRVFDSVSGKSIPRSEWGRIDKDGSNSKQSRTEWDYGEIHSIRGKSLTEAFAVEINDDFKLATKVGN

SEQ ID NO: 507 - DNA, 738 bp, Lactococcus lactis subsp. lactis
atgagaagatatttaatacttattgtggccttaatagggataacaggtttatcagggtgttatcaaacaagtcataaaaaggtgaggtttgacgaaggaagttatactaattttatttatgataataaatcgtatttcgtaactgataaggagattcctcaggagaacgttaacaattccaaagtaaaattttataagctgttgattgttgacatgaaaagtgagaaacttttatcaagtagcaacaaaaatagtgtgactttggtcttaaataatatttatgaggcttctgacaagtcgctatgtatgggtattaacgacagatactataagatacttccagaaagtgataagggggcggtcaaagctttgagattacaaaactttgatgtgacaagcgatatttctgatgataattttgttattgataaaaatgattcacgaaaaattgactatatgggaaatatttacagtatatcggacaccaccgtatctgatgaagaattgggagaatatcaggatgttttagctgaagtacgtgtgtttgattcagttagtggcaaaagtatcccgaggtctgaatgggggagaattgataaggatggttcaaattccaaacagagtaggacggaatgggattatggcgaaatccattctattagaggaaaatctcttactgaagcatttgccgttgagataaatgatgattttaagcttgcaacgaaggtaggaaactag

SEQ ID NO: 508 - protein, 261 aa, Rhizobium leguminosarum bv. trifolii
MNDEICLTGGGRTTVTRRGGVVYREGGPWSSTVISLLRHLEASGFAEAPSVVGTGFDERGRETLSFIEGEFVHPGPWSEEAFPQFGMMLRRLHDATASFKPPENSMWRDWFGRNLGEGQHVIGHCDTGPWNIVCRSGLPVGLIDWEVAGPVRADIELAQACWLNAQLYDDDIAERVGLGSVTMRAHQVRLLLDGYGLSRKQRGGFVDKLITFAVHDAAEQAKEAAVTPESNDAEPLWAIAWRTRSASWMLHHRQTLEAALA

SEQ ID NO: 509 - DNA, 786 bp, Rhizobium leguminosarum bv. trifolii
atgaatgatgagatttgcctgacaggtggcggacgaacgactgtcacgcggcgcggcggagtcgtgtatcgcgaaggcggcccgtggtcatcaaccgtcatttcgctcctgcggcatctggaagcctctggcttcgctgaagctccttccgttgtcggcaccggtttcgatgagcgcggccgggagacattatcgtttatcgagggtgagtttgttcacccaggcccttggtcggaggaggcttttccgcaatttggaatgatgttgcggcgactgcacgatgccaccgcctcgttcaaacctcccgaaaactcgatgtggcgcgattggttcgggcgtaacctcggtgagggtcaacacgtaataggacactgcgacacaggcccatggaacattgtttgccggtcaggattgcctgtcgggttgatagattgggaggtggctgggcctgtcagggcggatatcgaattggcccaggcttgttggctgaatgcccagctctacgatgacgacattgcggagagggtcggattaggctctgtgaccatgagagcgcatcaagttcgcctgctgcttgacggctatggtctgtctcggaagcaacgcggcggcttcgtcgacaagctaatcacgttcgcagttcacgatgcggccgagcaggcgaaagaggcggctgtcacgccagagtcgaacgatgcggaaccgctatgggcaattgcctggcgcactagaagtgcctcctggatgctccatcatcggcaaacactggaagcagcgctggcatag

SEQ ID NO: 510 - protein, 436 aa, Bacillus subtilis
MNNIIPIMSLLFKQLYSRQGKKDAIRIAAGLVILAVFEIGLIRQAGIDESVLRKTYIILALLLMNTYMVFLSVTSQWKESYMKLSCLLPISSRSFWLAQSVVLFVDTCLRRTLFFFILPLFLFGNGTLSGAQTLFWLGRFSFFTVYSIIFGVVLSNHFVKKKNLMFLLHAAIFACVCISAALMPAATIPLCAVHILWAVVIDFPVFLQAPPQQGKMHSFMRRSEFSFYKREWNRFISSKAMLLNYAVMAVFSGFFSFQMMNTGIFNQQVIYIVISALLLICSPIALLYSIEKNDRMLLITLPIKRKTMFWAKYRFYSGLLAGGFLLVVMIVGFISGRSISVLTFLQCIELLLAGAYIRLTADEKRPSFSWQTEQQLWSGFSKYRSYLFCLPLFLAILAGTAVSLAVIPIAGLVIVYYLQKQDGGFFDTSKRERLGS

SEQ ID NO: 511 - DNA, 1000 bp, Bacillus subtilis
atgaataacataatccctatcatgtctttgctgttcaaacagctttacagccggcaagggaaaaaggacgccatccgcattgccgcaggccttgtcattctggccgtgtttgaaatcgggctgatccgccaggccggcattgatgaatcggtgttgcgcaaaacgtatatcatactcgcgcttcttttgatgaacacatatatggtgtttctttccgtgacatcacaatggaaggaatcttatatgaagctgagctgcctgctgccgatttcttcacggagcttttggctcgcccagagtgtcgttttgtttgtcgatacctgtttgagaagaactttattcttttttattttaccgctgttcttatttggaaacggaacgctgtcaggggcgcaaacattgttttggctcggcaggttttcgttttttaccgtttactccattattttcggagttgtgctaagcaaccacttcgtcaaaaagaagaacttgatgtttctgctgcatgcggcgatattcgcctgtgtatgtatcagcgccgctttgatgccggccgccacgattccgctttgcgcggttcatatcctgtgggcggtggtcattgactttcctgtctttctgcaggcgcctccgcagcagggcaagatgcattcatttatgcggcgatctgaattttcgttttacaaaagagaatggaaccgatttatctcttctaaagcgatgctgttaaattacgcggtaatggcggtattcagcggcttcttttcgttccagatgatgaacaccggcatcttcaatcagcaagtgatttatatcgtgatttccgcgcttttgctcatctgctcgccgatcgcccttttgtattcgattgaaaaaaatgaccggatgctgctcatcacgcttccgatcaagcgaaaaacgatgttttgggcgaaatatcgcttttattcaggcctattggcaggcggatttctccttgtcgtgatgattgtgggtttca

SEQ ID NO: 512 - protein, 239 aa, Bacillus subtilis
MSILDIHDVSVWYERDNVILEQVDLHLEKGAVYGLLGVNGAGKTTLINTLTGVNRNFSGRFTLCGIEAEAGMPQKTSDQLKTHRYFAADYPLLFTEITAKDYVSFVHSLYQKDFSEQQFASLAEAFHFSKYINRRISELSLGNRQKVVLMTGLLLRAPLFILDEPLVGLDVESIEVFYQKMREYCEAGGTILFSSHLLDVVQRFCDYAAILHNKQIQKVIPIGEETDLRREFFEVIGHE

SEQ ID NO: 513 - DNA, 716 bp, Bacillus subtilis
gcattttggatatacacgatgtatccgtttggtatgaacgggacaacgtcatcttagagcacgtggacttacacttagaaaaaggcgccgtttacggattgcttggggtaaacggtgccggcaaaacaacactgatcaatacgctgacaggagtgaaccgcaattacagcgggggctttacgctgtgcggcattgaagctgaggccggcatgccgcagaaaacatcagatcaactgaagattcaccgttacttcgccgctgattatccgctgctgtttacagaaattacggcgaaggactatgtgtctttcgtccattcgctttatcaaaaggatttttcagagcgacagtttgccagtttggctgaggcctttcatttttcaaaatacatcaacaggagaatctcggagctgtccttggggaacaggcaaaaggttgtgttgatgacaggattattgctgcgggctcccctgtttattttggatgagccgctcgtcggtttggatgtggaatcaatagaggtcttttatcagaaaatgcgggagtactgtgaggaaggcggaaccattttgttttcttcccatctgctcgatgtcgtgcagagattttgtgattttgcggccattctgcacaacaaacagatccaaaaggtcattccgattggggaggagaccgatctgcggcgggaattttttgaggttatcggccatgaataa

SEQ ID NO: 514 - protein, 53 aa, Bacillus subtilis
MSPAQRRILLYILSFIFVIGAVVYFVKSDYLFTLIFIAIAILFGMRARKADSR

SEQ ID NO: 515 - DNA, 162 bp, Bacillus subtilis
ttgtcaccagcacaaagaagaattttactgtatatcctttcatttatctttgtcatcggcgcagtcgtctattttgtcaaaagcgattatctgtttacgctgattttcattgccattgccattctgttcgggatgcgcgcgcggaaggctgactcgcgatga

SEQ ID NO: 516 - protein, 87 aa, Escherichia coli
MELKNSISDYTEAEFVQLLKEIEKENVAATDDVLDVLLEHFVKITEHPDGTDLIYYPSDNRDDSPEGIVKEIKEWRAANGKPGFKQG

SEQ ID NO: 517 - DNA, 264 bp, Escherichia coli
atggaactgaaaaatagtattagtgattacacagaggctgagtttgttcaacttcttaaggaaattgaaaaagagaatgttgctgcaactgatgatgtgttagatgtgttactcgaacactttgtaaaaattactgagcatccagatggaacggatctgatttattatcctagtgataatagagacgatagccccgaagggattgtcaaggaaattaaagaatggcgagctgctaacggtaagccaggatttaaacagggctga

SEQ ID NO: 518 - protein, 87 aa, Pseudomonas aeruginosa
MKSKISEYTEKEFLEFVEDIYTNNKKKFPTEESHIQAVLEFKKLTEHPSGSDLLYYPNENREDSPAGVVKEVKEWRASKGLPGFKAG

SEQ ID NO: 519 - DNA, 264 bp, Pseudomonas aeruginosa
atgaagtccaagatttccgaatatacggaaaaagagtttcttgagtttgttgaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 520 - protein, 87 aa, Pseudomonas aeruginosa
MKSKISEYTEKEFLEFVKDIYTNNKKKFPTEESHIQAVLEFKKLTEHPSGSDLLYYPNENREDSPAGVVKEVKEWRASKGLPGFKAG

SEQ ID NO: 521 - DNA, 264 bp, Pseudomonas aeruginosa
atgaagtccaagatttccgaatatacggaaaaagagtttcttgagtttgttaaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 522 - protein, 95 aa, Enterococcus hirae
MDFTKEEKLLNAISKVYNEATIDDYPDLKEKLFLYSKEISEGKSVGEVSMKLSSFLGRYILKHKFGLPKSLIELQEIVSKESQVYRGWASIGIWS

SEQ ID NO: 523 - DNA, 288 bp, Enterococcus hirae
atggattttactaaagaagaaaaacttttaaatgcaattagtaaagtatacaatgaagcaactatagatgactatcctgacttaaaagaaaagctctttctttattctaaagaaatcagtgagggaaaaagtgttggtgaagttagtatgaaattaagtagttttcttggaagatatattttaaaacataaatttggattacctaaatctttaatagaattacaagaaattgttagtaaggaatctcaagtatatagaggatgggcttctattggtatttggagttaa

SEQ ID NO: 524 - protein, 113 aa, Leuconostoc mesenteroides
MKKKYRYLEDSKNYTSTLYSLLVDNVDKPGYSDICDVLLQVSKKLDNTQSVEALINRLVNYIRITASTYKIIFSKKEEELIIKLGVIGQKAGLNGQYMADFSDKSQFYSVFDQ

SEQ ID NO: 525 - DNA, 342 bp, Leuconostoc mesenteroides
ttgaaaaaaaagtatcggtatttagaagatagcaaaaattacactagtacactctattctctgttagttgataatgttgacaaacctggatactcagatatttgcgatgttttgcttcaagtttctaagaagttggataatactcaaagtgttgaagcgctaattaatcgattggttaattatattcgtattactgcttcaacatacaaaattattttttcaaaaaaagaagaggaattgattataaaacttggtgttattggacaaaaagctggacttaatggtcagtatatggctgatttttcagacaagtctcagttttacagcgttttcgatcagtaa

SEQ ID NO: 526 - protein, 93 aa, Escherichia coli
MSFLNFAFSPVFFSIMACYFIVWRNKRNEFVCNRLLSIIIISFLICFIYPWLNYKIEVKYYIFEQFYLFCFLSSLVAVVINLIVYFILYRRCI

SEQ ID NO: 527 - DNA, 282 bp, Escherichia coli
atgagttttcttaattttgcattttctcctgtattcttctccattatggcgtgttatttcattgtatggagaaataaacgaaacgaatttgtctgcaatagattgctatcaattataataatatcttttttgatatgcttcatatatccatggctaaattacaaaatcgaagttaaatattatatatttgaacagttttatcttttttgttttttatcgtcactcgtggctgttgtaataaacctaattgtatactttatattatacaggagatgtatatga

SEQ ID NO: 528 - protein, 96 aa, Escherichia coli
MHLKYYLHNLPESLIPWILILIFNDNDNTPLLFIFISSIHVLLYPYSKLTISRYIKENTKLKKEPWYLCKLSALFYLLMAIPVGLPSFIYYTLKRN

SEQ ID NO: 529 - DNA, 291 bp, Escherichia coli
atgcatttaaaatactacctacataatttacctgaatcacttataccatggattcttattttaatatttaacgacaatgataacactcctttgttatttatatttatatcatcaatacatgtattgctatatccatactctaaattaaccatatctagatatatcaaagaaaatacaaagttaaaaaaagaaccctggtacttatgcaagttatctgcattgttttatttattaatggcaatcccagtaggattgccaagtttcatatattacactctaaagagaaattaa

SEQ ID NO: 530 - protein, 344 aa, Escherichia coli
MMIQSHPLLAAPLAVGDTIGFFSSSAPATVTAKNRFFRGVEFLQRKGFKLVSGKLTGKTDFYRSGTIKERAQEFNELVYNPDITCIMSTIGGDNSNSLLPFLDYDAIIANPKIIIGYSDTTALLAGIYAKTGLITFYGPALIPSFGEHPPLVDITYESFIKILTRKQSGIYTYTLPEKWSDESINWNENKILRPKKLYKNNCAFYGSGKVEGRVIGGNLNTLTGIWGSEWMPEILNGDILFIEDSRKSIATIERLFSMLKLNRVFDKVSAIILGKHELFDCAGSKRRPYEVLTEVLDGKQIPVLDGFDCSHTHPMLTLPLGVKLAIDFDNKNISITEQYLSTEK

SEQ ID NO: 531 - DNA, 1000 bp, Escherichia coli
atgatgatacaatctcatccactactggccgctcccctggcagtaggagatacaattggtttcttttcatcatctgctccggcaacagttactgcaaaaaatcgtttttttcggggagttgagtttcttcagagaaagggatttaagctggtatcagggaagcttaccggtaaaacagatttttatcgttcaggtactattaaagaaagagctcaagaatttaatgagttagtctacaatcctgatattacctgtataatgtcaacgatcggtggagataacagtaattcactactaccgtttctggactatgatgctatcattgcaaaccccaaaattatcataggttactcagatacaactgctttattagcaggaatatatgcaaaaacagggttaataacattctatggaccagctcttattccttcgtttggtgaacatccacctcttgtggatataacatatgaatcatttattaaaatactaacaagaaaacaatcaggaatatatacctacacattacctgaaaagtggagtgatgagagcataaactggaatgaaaacaagatattaaggcctaagaagctatataaaaacaactgtgccttttatggttccggaaaagttgaggggcgtgtaattggaggaaatctaaatactttgacaggtatatgggggagtgaatggatgcctgaaattcttaatggagatatattgtttattgaggacagtcggaaaagcattgcaacaattgaacgattattctctatgctaaagcttaatcgcgtgtttgataaagttagtgcaataatactcgggaaacatgagctttttgattgtgcaggaagtaaacgcagaccatatgaagtattaacagaggtattagatgggaaacagattcctgtactggatggatttgattgttcacatacacatccaatgctaactcttccacttggtgtaaaattagctattgactttgacaacaaaaatatat

SEQ ID NO: 532 - protein, 90 aa, Lactobacillus sakei
MKADYKKINSILTYTSTALKNPKIIKDKDLVVLLTIIQEEAKQNRIFYDYKRKFRPAVTRFTIDNNFEIPDCLVKLLSAVETPKAWSGFS

SEQ ID NO: 533 - DNA, 268 bp, Lactobacillus sakei
ggcagattataaaaaaataaattcaatactaacttacacatctactgctttaaaaaaccctaaaattataaaagataaagatttagtagtccttctaactattattcaagaagaagccaaacaaaatagaatcttttatgattataaaagaaaatttcgtccagcggttactcgctttacaattgataataattttgagattcctgattgtttggttaaactactgtcagctgttgaaacacctaaggcgtggtctggatttagttag

SEQ ID NO: 534 - protein, 83 aa, Escherichia coli
MKLSPKAAIEVCNEAAKKGLWILGIDGGHWLNPGFRIDSSASWTYDMPEEYKSKTPENNRLAIENIKDDIENGYTAFIITLKM

SEQ ID NO: 535 - DNA, 251 bp, Escherichia coli
tgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggaggaatacaaatcaaaaacccctgaaaataatagattggctattgaaaatattaaagatgatattgagaatggatacactgctttcattatcacgttaaagatgtaa

SEQ ID NO: 536 - protein, 436 aa, Bacillus subtilis
MNNIFPIMSLLFKQLYSRQGKKDAIRIAAGLVILAVFEIGLIRQAGIDESVLGKTYIILALLLMNTYMVFLSVTSQWKESYMKLSCLLPISSRSFWLAQSVVLFVDTCLRRTLFFFILPLFLFGNGTLSGAQTLFWLGRFSFFTVYSILFGVMLSNHFVKKKNSMFLLHAAVFAFVCLSAAFMPAVTIPLCAVHMLWAVIIDFPVFLQAPPHQSKMHFFMRRSEFSFYKREWNRFISSKAMLLNYVVMAAFSGFFSFQMMNTGIFNQQVIYIVISALLLICSPIALLYSIEKNDRMLLITLPIKRRTMFWAKYRFYSGLLAGGFLLVAIIVGFISGRPISALTFVQCMELLLAGAFIRLTADEKRPSFGWQTEQQLWSGFSKYRSYLFCLPLFLATLAGTAVSLAVIPIAALIIVYYLQKQDGGFFDTSKRERIGS

SEQ ID NO: 537 - DNA, 1000 bp, Bacillus subtilis
ttggggaggagaccgatctgcggcgggaattttttgaggttatcggccatgaataacatattccccatcatgtcgttgctgttcaaacagctgtacagccggcaagggaaaaaggacgctatccgcattgctgcagggcttgtgattctcgccgtgtttgaaatcgggctgatccgacaagccggcattgacgaatcggtgttgggaaaaacgtatatcatattggcgcttctcttaatgaacacgtatatggtgtttctttccgtgacatcacaatggaaggaatcttatatgaagctgagctgtctgctgccgatttcatcacggagcttttggctcgcccagagtgtcgttctgtttgtcgatacctgtttgagaagaacgttattcttttttattttaccgctgttcttatttggaaacggaacgctgtcaggggcgcaaacattgttttggcttggcagattttcgttttttaccgtttactcgattctattcggagttatgctaagcaaccatttcgtcaaaaagaagaactcgatgtttctgctgcatgcggcggtattcgcctttgtatgcctcagtgccgcttttatgccggccgtcacgatcccgctatgcgcggttcacatgctatgggcggtgatcattgactttccggtctttctgcaggcgcctccgcatcagagcaagatgcatttttttatgcggcgatctgaattttcgttttacaaaagagaatggaaccgatttatttcttctaaagcgatgctgttaaattacgtggtgatggcggcgttcagcggattcttttcgttccagatgatgaacactggcatcttcaatcagcaagtgatttatattgtgatttccgctctattgctgatttgctcgccgatcgcccttttgtactctattgaaaaaaacgatcgcatgctgctcatcacgcttccaattaaaagaagaacgatgttttgggcgaaatatcgcttttattcag

SEQ ID NO: 538 - protein, 580 aa, Escherichia coli
MERKQKNSLFNYIYSLMDVRGKFLFFSMLFITSLSSIIISISPLILAKITDLLSGSLSNFSYEYLVLLACLYMFCVISNKASVFLFMILQSSLRINMQKKMSLKYLRELYNENITNLSKNNAGYTTQSLNQASNDIYILVRNVSQNILSPVIQLISTIVVVLSTKDWFSAGVFFLYILVFVIFNTRLTGSLASLRKHSMDITLNSYSLLSDTVDNMIAAKKNNALRLISERYEDALTQENNAQKKYWLLSSKVLLLNSLLAVILFGSVFIYNILGVLNGVVSIGHFIMITSYIILLSTPVENIGALLSEIRQSMSSLAGFIQRHAENKATSPSIPFLNMERKLNLSIRELSFSYSDDKKILNSVSLDLFTGKMYSLTGPSGSGKSTLVKIISGYYKNYFGDIYLNDISLRNISDEDLNDAIYYLTQDDYIFMDTLRFNLRLANYDASENEIFKVLKLANLSVVNNEPVSLDTHLINRGNNYSGGQKQRISLARLFLRKPAIIIIDEATSALDYINESEILSSIRTHFPDALIINISHRINLLECSDCVYVLNEGNIVASGHFRDLMVSNEYISGLASVTE

SEQ ID NO: 539 - DNA, 1000 bp, Escherichia coli
atggaaagaaaacagaaaaactcattatttaattatatttattcattaatggatgtaagaggtaaatttttattcttttccatgttattcattacatcattatcatcgataatcatatctatttcaccattgattcttgcaaagattacagatttactgtctggctcattgtcaaattttagttatgaatatctggttttacttgcctgtttatacatgttttgcgttatatctaataaagcaagtgtttttttatttatgatactgcaaagtagtctacgtattaacatgcagaaaaaaatgtcgctaaagtatttgagagaattgtataacgaaaatataactaacttgagtaaaaataatgctggatatacaacgcaaagtcttaaccaggcttcaaatgacatttatattcttgtgagaaatgtttcccagaatatcctgtcacctgttatacaacttatttccactattgttgttgttttatctacgaaggactggttttctgccggtgtgttttttctctatattctggtatttgtaatttttaataccagactgactggcagtttagcgtctctcagaaaacacagcatggatatcactcttaactcttatagtctgttatctgatactgttgataacatgatagcagctaaaaagaataatgcattaagacttatttctgaacgttatgaagatgctctcactcaggaaaacaatgctcagaaaaaatactggttactcagttctaaagttcttttattgaactctttacttgctgtaatattatttggttctgtattcatatataatattttaggtgtgctgaatggtgtagttagtatcggccacttcattatgattacatcatatatcattcttctttcaacgccagtggaaaatataggggcattgctaagtgagatcaggcagtcaatgtctagcctggcaggttttattcaacgtcatgccgagaataaagccacatctccttcaa

SEQ ID NO: 540 - protein, 95 aa, Klebsiella pneumoniae
MTLLSFGFSPVFFSVMAFCIISRSKFYPQRTRNKVIVLILLTFFICFLYPLTKVYLVGSYGIFDKFYLFCFISTLIAIAINVVILTINGAKNERN

SEQ ID NO: 541 - DNA, 288 bp, Klebsiella pneumoniae
atgacattactttcatttggattttctcctgttttcttttcagtcatggcgttctgtatcatttcacgtagtaaattctatccgcagagaacgcgaaacaaagttattgttctgattttactaactttttttatttgttttttatatccattaacaaaagtgtatctggtgggaagttacggtatatttgacaaattctacctcttttgctttatttctacgttaattgcaatagcaattaacgtagtgatacttacaataaatggagctaagaatgagagaaattag

SEQ ID NO: 542 - RNA, 13 nt, Artificial Sequence
  Kozak sequence
gccgccrccaugg

SEQ ID NO: 543 - RNA, 6 nt, Artificial Sequence
  Shine-Delgarno sequence
ggaggu

SEQ ID NO: 544 - DNA, 30 bp, Artificial Sequence
  Lead promoter
gaaaaccttgtcaatgaagagcgatctatg

SEQ ID NO: 545 - DNA, 30 bp, Artificial Sequence
  FecA promoter
ttctcgttcgactcatagctgaacacaaca

SEQ ID NO: 546 - DNA, 16 bp, Artificial Sequence
  Cu-sensitive promoter
atgacaaaattgtcat

SEQ ID NO: 547 - DNA, 30 bp, Artificial Sequence
  Fe promoter
accaatgctgggaacggccagggcacctaa

SEQ ID NO: 548 - DNA, 30 bp, Artificial Sequence
  Fe and UV promoters
ctgaaagcgcataccgctatggagggggtt

SEQ ID NO: 549 - DNA, 30 bp, Artificial Sequence
  PrFe (PI + PII rus operon)
tagatatgcctgaaagcgcataccgctatg

SEQ ID NO: 550 - DNA, 30 bp, Artificial Sequence
  Lux cassette right promoter
tgttatagtcgaatacctctggcggtgata

SEQ ID NO: 551 - DNA, 30 bp, Artificial Sequence
  P(Las) TetO
ttttggtacactccctatcagtgatagaga

SEQ ID NO: 552 - DNA, 30 bp, Artificial Sequence
  P(Las) CIO
ctttttggtacactacctctggcggtgata

SEQ ID NO: 553 - DNA, 30 bp, Artificial Sequence
  P(Rhl)
tacgcaagaaaatggtttgttatagtcgaa

SEQ ID NO: 554 - DNA, 30 bp, Artificial Sequence
  Double Promoter (LuxR/HSL, positive / cI, negative)
cgtgcgtgttgataacaccgtgcgtgttga

SEQ ID NO: 555 - DNA, 30 bp, Artificial Sequence
  P2 promoter in agr operon from S. aureus
agattgtactaaatcgtataatgacagtga

SEQ ID NO: 556 - DNA, 30 bp, Artificial Sequence
  plux-cI hybrid promoter
gtgttgatgcttttatcaccgccagtggta

SEQ ID NO: 557 - DNA, 30 bp, Artificial Sequence
  plux-lac hybrid promoter
agtgtgtggaattgtgagcggataacaatt

SEQ ID NO: 558 - DNA, 30 bp, Artificial Sequence
  CinR, CinL and glucose controlled promotor
acatcttaaaagttttagtatcatattcgt

SEQ ID NO: 559 - DNA, 30 bp, Artificial Sequence; identical to SEQ ID NO: 553
  RhIR promoter repressible by CI

SEQ ID NO: 560 - DNA, 30 bp, Artificial Sequence
  Reverse Lux Promoter
tcttgcgtaaacctgtacgatcctacaggt

SEQ ID NO: 561 - DNA, 30 bp, Artificial Sequence
  rhlI promoter
atcctcctttagtcttccccctcatgtgtg

SEQ ID NO: 562 - DNA, 30 bp, Artificial Sequence
  lasI promoter
taaaattatgaaatttgcataaattcttca

SEQ ID NO: 563 - DNA, 30 bp, Artificial Sequence
  LuxR+3OC6HSL independent R0065
gtgttgactattttacctctggcggtgata

SEQ ID NO: 564 - DNA, 30 bp, Artificial Sequence
  LasR/LasI Inducible & RHLR/RHLI repressible Promoter
gaaatctggcagtttttggtacacgaaagc

SEQ ID NO: 565 - DNA, 30 bp, Artificial Sequence
  pLux/cI Hybrid Promoter
acaccgtgcgtgttgatatagtcgaataaa

SEQ ID NO: 566 - DNA, 30 bp, Artificial Sequence
  pLas promoter
aaaattatgaaatttgtataaattcttcag

SEQ ID NO: 567 - DNA, 30 bp, Artificial Sequence
  pLas/cI Hybrid Promoter
ggttctttttggtacctctggcggtgataa

SEQ ID NO: 568 - DNA, 30 bp, Artificial Sequence
  pLas/Lux Hybrid Promoter
tgtaggatcgtacaggtataaattcttcag

SEQ ID NO: 569 - DNA, 30 bp, Artificial Sequence
  pLux
caagaaaatggtttgttatagtcgaataaa

SEQ ID NO: 570 - DNA, 30 bp, Artificial Sequence
  pLux/Las Hybrid Promoter
ctatctcatttgctagtatagtcgaataaa

SEQ ID NO: 571 - DNA, 30 bp, Artificial Sequence
  Hybrid promoter HSL-LuxR activated, P22 C2 repressed
tagtttataatttaagtgttctttaatttc

SEQ ID NO: 572 - DNA, 30 bp, Artificial Sequence
  PAI+LasR -> LuxI (AI)
caccttcgggtgggcctttctgcgtttata

SEQ ID NO: 573 - DNA, 30 bp, Artificial Sequence
  PAI+LasR -> LasI & AI+LuxR --| LasI
aataactctgatagtgctagtgtagatctc

SEQ ID NO: 574 - DNA, 30 bp, Artificial Sequence; identical to SEQ ID NO: 572
  PAI+LasR -> LasI+GFP & AI+LuxR --| LasI+GFP

SEQ ID NO: 575 - DNA, 30 bp, Artificial Sequence; identical to SEQ ID NO: 572
  Complex QS -> LuxI & LasI circuit

SEQ ID NO: 576 - DNA, 30 bp, Artificial Sequence; identical to SEQ ID NO: 569
  position 3 mutated promoter lux pR-3 (luxR & HSL regulated)

SEQ ID NO: 577 - DNA, 30 bp, Artificial Sequence; identical to SEQ ID NO: 569
  position 5 mutated promoter lux pR-5 (luxR & HSL regulated)

SEQ ID NO: 578 - DNA, 30 bp, Artificial Sequence; identical to SEQ ID NO: 569
  position 3&5 mutated promoter lux pR-3/5 (luxR & HSL regulated)

SEQ ID NO: 579 - DNA, 30 bp, Artificial Sequence
  Promoter (HSL-mediated luxR repressor)
ttgacacctgtaggatcgtacaggtataat

SEQ ID NO: 580 - DNA, 30 bp, Artificial Sequence; identical to SEQ ID NO: 569
  Promoter (luxR & HSL regulated -- lux pR)

SEQ ID NO: 581 - DNA, 30 bp, Artificial Sequence
  Promoter (luxR & HSL regulated -- lux pL)
cacgcaaaacttgcgacaaacaataggtaa

SEQ ID NO: 582 - DNA, 30 bp, Artificial Sequence
  Promoter (RhlR & C4-HSL regulated)
gttagctttcgaattggctaaaaagtgttc

SEQ ID NO: 583 - DNA, 30 bp, Artificial Sequence
  Promoter (cinR and HSL regulated)
ccattctgctttccacgaacttgaaaacgc

SEQ ID NO: 584 - DNA, 30 bp, Artificial Sequence
  Promoter (LasR & PAI regulated)
ggccgcgggttctttttggtacacgaaagc

SEQ ID NO: 585 - DNA, 30 bp, Artificial Sequence
  Promoter, Standard (luxR and HSL regulated -- lux pR)
aagaaaatggtttgttgatactcgaataaa

SEQ ID NO: 586 - DNA, 30 bp, Artificial Sequence
  P(Bla)
gtttatacataggcgagtactctgttatgg

SEQ ID NO: 587 - DNA, 30 bp, Artificial Sequence
  P(Cat)
agaggttccaactttcaccataatgaaaca

SEQ ID NO: 588 - DNA, 30 bp, Artificial Sequence
  P(Kat)
taaacaactaacggacaattctacctaaca

SEQ ID NO: 589 - DNA, 30 bp, Artificial Sequence
  Template for Building Primer Family Member
acatcaagccaaattaaacaggattaacac

SEQ ID NO: 590 - DNA, 30 bp, Artificial Sequence
  Reverse lambda cI-regulated promoter
gaggtaaaatagtcaacacgcacggtgtta

SEQ ID NO: 591 - DNA, 30 bp, Artificial Sequence
  Key Promoter absorbs 3
caggccggaataactccctataatgcgcca

SEQ ID NO: 592 - DNA, 30 bp, Artificial Sequence
  constitutive promoter family member
ggctagctcagtcctaggtacagtgctagc

SEQ ID NO: 593 - DNA, 30 bp, Artificial Sequence
  constitutive promoter family member
agctagctcagtcctaggtattatgctagc

SEQ ID NO: 594 - DNA, 30 bp, Artificial Sequence
  constitutive promoter family member
agctagctcagtcctaggtactgtgctagc

SEQ ID NO: 595 - DNA, 30 bp, Artificial Sequence
  constitutive promoter family member
agctagctcagtcctagggattatgctagc

SEQ ID NO: 596 - DNA, 30 bp, Artificial Sequence
  constitutive promoter family member
agctagctcagtcctaggtattgtgctagc

SEQ ID NO: 597 - DNA, 30 bp, Artificial Sequence
  constitutive promoter family member
ggctagctcagtcctaggtactatgctagc

SEQ ID NO: 598 - DNA, 30 bp, Artificial Sequence
  constitutive promoter family member
ggctagctcagtcctaggtatagtgctagc

SEQ ID NO: 599 - DNA, 30 bp, Artificial Sequence
  constitutive promoter family member
ggctagctcagccctaggtattatgctagc

SEQ ID NO: 600 - DNA, 30 bp, Artificial Sequence
  constitutive promoter family member
agctagctcagtcctaggtataatgctagc

SEQ ID NO: 601 - DNA, 30 bp, Artificial Sequence
  constitutive promoter family member
agctagctcagtcctagggactgtgctagc

SEQ ID NO: 602 - DNA, 30 bp, Artificial Sequence
  constitutive promoter family member
ggctagctcagtcctaggtacaatgctagc

SEQ ID NO: 603 - DNA, 30 bp, Artificial Sequence; identical to SEQ ID NO: 598
  constitutive promoter family member

SEQ ID NO: 604 - DNA, 30 bp, Artificial Sequence; identical to SEQ ID NO: 595
  constitutive promoter family member

SEQ ID NO: 605 - DNA, 30 bp, Artificial Sequence
  constitutive promoter family member
ggctagctcagtcctagggattatgctagc

SEQ ID NO: 606 - DNA, 30 bp, Artificial Sequence; identical to SEQ ID NO: 602
  constitutive promoter family member

SEQ ID NO: 607 - DNA, 30 bp, Artificial Sequence
  constitutive promoter family member
agctagctcagcccttggtacaatgctagc

SEQ ID NO: 608 - DNA, 30 bp, Artificial Sequence
  constitutive promoter family member
agctagctcagtcctagggactatgctagc

SEQ ID NO: 609 - DNA, 30 bp, Artificial Sequence
  constitutive promoter family member
agctagctcagtcctagggattgtgctagc

SEQ ID NO: 610 - DNA, 30 bp, Artificial Sequence
  constitutive promoter family member
ggctagctcagtcctaggtattgtgctagc

SEQ ID NO: 611 - DNA, 30 bp, Artificial Sequence; identical to SEQ ID NO: 600
  constitutive promoter family member

SEQ ID NO: 612 - DNA, 30 bp, Artificial Sequence
  1bp mutant from J23107
ggctagctcagtcctaggtattatgctagc

SEQ ID NO: 613 - DNA, 30 bp, Artificial Sequence; identical to SEQ ID NO: 602
  1bp mutant from J23114

SEQ ID NO: 614 - DNA, 30 bp, Artificial Sequence
  pBAD reverse
aaagtgtgacgccgtgcaaataatcaatgt

SEQ ID NO: 615 - DNA, 30 bp, Artificial Sequence
  NikR promoter, a protein of the ribbon helix- helix family of trancription factors that repress expre
gacgaatacttaaaatcgtcatacttattt

SEQ ID NO: 616 - DNA, 30 bp, Artificial Sequence
  lacq_Promoter
aaacctttcgcggtatggcatgatagcgcc

SEQ ID NO: 617 - DNA, 30 bp, Artificial Sequence
  lacIQ - promoter sequence
tgatagcgcccggaagagagtcaattcagg

SEQ ID NO: 618 - DNA, 30 bp, Artificial Sequence
  E. Coli CreABCD phosphate sensing operon promoter
ttatttaccgtgacgaactaattgctcgtg

SEQ ID NO: 619 - DNA, 30 bp, Artificial Sequence
  GlnRS promoter
catacgccgttatacgttgtttacgctttg

SEQ ID NO: 620 - DNA, 30 bp, Artificial Sequence
  Constitutive weak promoter of lacZ
ttatgcttccggctcgtatgttgtgtggac

SEQ ID NO: 621 - DNA, 30 bp, Artificial Sequence
  Mutated LacZ promoter
ttatgcttccggctcgtatggtgtgtggac

SEQ ID NO: 622 - DNA, 30 bp, Artificial Sequence
  constitutive promoter with (TA)10 between -10 and -35 elements
atatatatatatatataatggaagcgtttt

SEQ ID NO: 623 - DNA, 30 bp, Artificial Sequence; identical to SEQ ID NO: 622
  constitutive promoter with (TA)9 between -10 and -35 elements

SEQ ID NO: 624 - DNA, 30 bp, Artificial Sequence
  constitutive promoter with (C)10 between -10 and -35 elements
ccccgaaagcttaagaatataattgtaagc

SEQ ID NO: 625 - DNA, 30 bp, Artificial Sequence; identical to SEQ ID NO: 624
  constitutive promoter with (C)12 between -10 and -35 elements

SEQ ID NO: 626 - DNA, 30 bp, Artificial Sequence
  optimized (TA) repeat constitutive promoter with 13 bp between -10 and -35 elements
tgacaatatatatatatatataatgctagc

SEQ ID NO: 627 - DNA, 30 bp, Artificial Sequence
  optimized (TA) repeat constitutive promoter with 15 bp between -10 and -35 elements
acaatatatatatatatatataatgctagc

SEQ ID NO: 628 - DNA, 30 bp, Artificial Sequence
  optimized (TA) repeat constitutive promoter with 17 bp between -10 and -35 elements
aatatatatatatatatatataatgctagc

SEQ ID NO: 629 - DNA, 30 bp, Artificial Sequence
  optimized (TA) repeat constitutive promoter with 19 bp between -10 and -35 elements
tatatatatatatatatatataatgctagc

SEQ ID NO: 630 - DNA, 30 bp, Artificial Sequence; identical to SEQ ID NO: 629
  optimized (TA) repeat constitutive promoter with 21 bp between -10 and -35 elements

SEQ ID NO: 631 - DNA, 30 bp, Artificial Sequence
  optimized (A) repeat constitutive promoter with 17 bp between -10 and -35 elements
aaaaaaaaaaaaaaaaaatataatgctagc

SEQ ID NO: 632 - DNA, 30 bp, Artificial Sequence; identical to SEQ ID NO: 631
  optimized (A) repeat constitutive promoter with 18 bp between -10 and -35 elements

SEQ ID NO: 633 - DNA, 30 bp, Artificial Sequence; identical to SEQ ID NO: 572
  J23101GFP

SEQ ID NO: 634 - DNA, 30 bp, Artificial Sequence; identical to SEQ ID NO: 572
  J23119IFP

SEQ ID NO: 635 - DNA, 30 bp, Artificial Sequence; identical to SEQ ID NO: 572
  J23119HO1

SEQ ID NO: 636 - DNA, 30 bp, Artificial Sequence; identical to SEQ ID NO: 572
  Infrared signal reporter (J23119IFPJ23119 HO1)

SEQ ID NO: 637 - DNA, 30 bp, Artificial Sequence; identical to SEQ ID NO: 592
  Double terminator + constitutive promoter

SEQ ID NO: 638 - DNA, 30 bp, Artificial Sequence
  Double terminator + Constitutive promoter + Strong RBS
tgctagctactagagattaaagaggagaaa

SEQ ID NO: 639 - DNA, 30 bp, Artificial Sequence
  IPTG inducible Lac promoter cassette
ttgtgagcggataacaagatactgagcaca

SEQ ID NO: 640 - DNA, 30 bp, Artificial Sequence; identical to SEQ ID NO: 639
  IPTG inducible Lac promoter cassette

SEQ ID NO: 641 - DNA, 30 bp, Artificial Sequence; identical to SEQ ID NO: 639
  IPTG inducible Lac promoter cassette

SEQ ID NO: 642 - DNA, 30 bp, Artificial Sequence
  M13K07 gene I promoter
cctgtttttatgttattctctctgtaaagg

SEQ ID NO: 643 - DNA, 30 bp, Artificial Sequence
  M13K07 gene II promoter
aaatatttgcttatacaatcttcctgtttt

SEQ ID NO: 644 - DNA, 30 bp, Artificial Sequence
  M13K07 gene III promoter
gctgataaaccgatacaattaaaggctcct

SEQ ID NO: 645 - DNA, 30 bp, Artificial Sequence
  M13K07 gene IV promoter
ctcttctcagcgtcttaatctaagctatcg

SEQ ID NO: 646 - DNA, 30 bp, Artificial Sequence
  M13K07 gene V promoter
atgagccagttcttaaaatcgcataaggta

SEQ ID NO: 647 - DNA, 30 bp, Artificial Sequence
  M13K07 gene VI promoter
ctattgattgtgacaaaataaacttattcc

SEQ ID NO: 648 - DNA, 30 bp, Artificial Sequence
  M13K07 gene VIII promoter
gtttcgcgcttggtataatcgctgggggtc

SEQ ID NO: 649 - DNA, 30 bp, Artificial Sequence
  M13110
ctttgcttctgactataatagtcagggtaa

SEQ ID NO: 650 - DNA, 30 bp, Artificial Sequence
  Modified promoter sequence of g3.
aaaccgatacaattaaaggctcctgctagc

SEQ ID NO: 651 - DNA, 30 bp, Artificial Sequence
  Constitutive Promoter I
caccacactgatagtgctagtgtagatcac

SEQ ID NO: 652 - DNA, 30 bp, Artificial Sequence
  Constitutive Promoter II
gccggaataactccctataatgcgccacca

SEQ ID NO: 653 - DNA, 30 bp, Artificial Sequence
  --Specify Parts List--
ttgacaagcttttcctcagctccgtaaact

SEQ ID NO: 654 - DNA, 30 bp, Artificial Sequence
  Full-length stationary phase osmY promoter
ggtttcaaaattgtgatctatatttaacaa

SEQ ID NO: 655 - DNA, 30 bp, Artificial Sequence; identical to SEQ ID NO: 654
  Minimal stationary phase osmY promoter

SEQ ID NO: 656 - DNA, 30 bp, Artificial Sequence
  htpG Heat Shock Promoter
tctattccaataaagaaatcttcctgcgtg

SEQ ID NO: 657 - DNA, 30 bp, Artificial Sequence
  Promoter veg a constitutive promoter for B. subtilis
aaaaatgggctcgtgttgtacaataaatgt

SEQ ID NO: 658 - DNA, 30 bp, Artificial Sequence
  Promoter 43 a constitutive promoter for B. subtilis
aaaaaaagcgcgcgattatgtaaaatataa

SEQ ID NO: 659 - DNA, 30 bp, Artificial Sequence
  Strong constitutive promoter for Bacillus subtilis
aattgcagtaggcatgacaaaatggactca

SEQ ID NO: 660 - DNA, 30 bp, Artificial Sequence
  PliaG
caagcttttcctttataatagaatgaatga

SEQ ID NO: 661 - DNA, 30 bp, Artificial Sequence
  PlepA
tctaagctagtgtattttgcgtttaatagt

SEQ ID NO: 662 - DNA, 30 bp, Artificial Sequence
  Pveg
aatgggctcgtgttgtacaataaatgtagt

SEQ ID NO: 663 - DNA, 30 bp, Artificial Sequence
  Promoter ctc for B. subtilis
atccttatcgttatgggtattgtttgtaat

SEQ ID NO: 664 - DNA, 30 bp, Artificial Sequence
  Promoter gsiB for B. subtilis
taaaagaattgtgagcgggaatacaacaac

SEQ ID NO: 665 - DNA, 30 bp, Artificial Sequence; identical to SEQ ID NO: 658
  Promoter 43 a constitutive promoter for B. subtilis

SEQ ID NO: 666 - DNA, 30 bp, Artificial Sequence
  Pspv2 from Salmonella
tacaaaataattcccctgcaaacattatca

SEQ ID NO: 667 - DNA, 30 bp, Artificial Sequence
  Pspv from Salmonella
tacaaaataattcccctgcaaacattatcg

SEQ ID NO: 668 - DNA, 30 bp, Artificial Sequence
  T7 promoter (strong promoter from T7 bacteriophage)
agggaatacaagctacttgttctttttgca

SEQ ID NO: 669 - DNA, 23 bp, Artificial Sequence
  T7 Promoter
taatacgactcactatagggaga

SEQ ID NO: 670 - DNA, 28 bp, Artificial Sequence
  T7 Promoter
gaatttaatacgactcactatagggaga

SEQ ID NO: 671 - DNA, 19 bp, Artificial Sequence
  T7 consensus -10 and rest
taatacgactcactatagg

SEQ ID NO: 672 - DNA, 30 bp, Artificial Sequence
  overlapping T7 promoter
gagtcgtattaatacgactcactatagggg

SEQ ID NO: 673 - DNA, 30 bp, Artificial Sequence
  more overlapping T7 promoter
agtgagtcgtactacgactcactatagggg

SEQ ID NO: 674 - DNA, 30 bp, Artificial Sequence
  weaken overlapping T7 promoter
gagtcgtattaatacgactctctatagggg

SEQ ID NO: 675 - DNA, 23 bp, Artificial Sequence
  T7 Consensus Promoter Sequence
taatacgactcactatagggaga

SEQ ID NO: 676 - DNA, 23 bp, Artificial Sequence
  T7 RNAP promoter
ttatacgactcactatagggaga

SEQ ID NO: 677 - DNA, 23 bp, Artificial Sequence
  T7 RNAP promoter
gaatacgactcactatagggaga

SEQ ID NO: 678 - DNA, 23 bp, Artificial Sequence
  T7 RNAP promoter
taatacgtctcactatagggaga

SEQ ID NO: 679 - DNA, 23 bp, Artificial Sequence
  T7 RNAP promoter
tcatacgactcactatagggaga

SEQ ID NO: 680 - DNA, 30 bp, Artificial Sequence
  T7 strong promoter
taatacgactcactatagggagaccacaac

SEQ ID NO: 681 - DNA, 30 bp, Artificial Sequence
  T7 weak binding and processivity
taattgaactcactaaagggagaccacagc

SEQ ID NO: 682 - DNA, 30 bp, Artificial Sequence
  T7 weak binding promoter
cgaagtaatacgactcactattagggaaga

SEQ ID NO: 683 - DNA, 30 bp, Artificial Sequence
  pCyc (Medium) Promoter
acaaacacaaatacacacactaaattaata

SEQ ID NO: 684 - DNA, 30 bp, Artificial Sequence
  pAdh (Strong) Promoter
ccaagcatacaatcaactatctcatataca

SEQ ID NO: 685 - DNA, 30 bp, Artificial Sequence
  pSte5 (Weak) Promoter
gatacaggatacagcggaaacaacttttaa

SEQ ID NO: 686 - DNA, 30 bp, Artificial Sequence
  yeast ADH1 promoter
tttcaagctataccaagcatacaatcaact

SEQ ID NO: 687 - DNA, 30 bp, Artificial Sequence
  cyc100 minimal promoter
cctttgcagcataaattactatacttctat

SEQ ID NO: 688 - DNA, 30 bp, Artificial Sequence; identical to SEQ ID NO: 687
  cyc70 minimal promoter

SEQ ID NO: 689 - DNA, 30 bp, Artificial Sequence; identical to SEQ ID NO: 687
  cyc43 minimal promoter

SEQ ID NO: 690 - DNA, 30 bp, Artificial Sequence; identical to SEQ ID NO: 687
  cyc28 minimal promoter

SEQ ID NO: 691 - DNA, 30 bp, Artificial Sequence; identical to SEQ ID NO: 687
  cyc16 minimal promoter

SEQ ID NO: 692 - DNA, 30 bp, Artificial Sequence
  pPGK1
ttatctactttttacaacaaatataaaaca

SEQ ID NO: 693 - DNA, 30 bp, Artificial Sequence; identical to SEQ ID NO: 683
  pCYC Yeast Promoter

SEQ ID NO: 694 - DNA, 30 bp, Artificial Sequence
  Yeast GPD (TDH3) Promoter
gtttcgaataaacacacataaacaaacaaa

SEQ ID NO: 695 - DNA, 30 bp, Artificial Sequence; identical to SEQ ID NO: 684
  yeast mid-length ADH1 promoter

SEQ ID NO: 696 - DNA, 30 bp, Artificial Sequence
  Yeast CLB1 promoter region, G2/M cell cycle specific
accatcaaaggaagctttaatcttctcata

SEQ ID NO: 697 - DNA, 30 bp, Artificial Sequence
  CMV promoter
agaacccactgcttactggcttatcgaaat

SEQ ID NO: 698 - DNA, 30 bp, Artificial Sequence
  Ubc Promoter
ggccgtttttggcttttttgttagacgaag

SEQ ID NO: 699 - protein, 9 aa, Lactobacillus curvatus
NIPQLTPTP

SEQ ID NO: 700 - DNA, 27 bp, Lactobacillus curvatus
aacattccgcagctgaccccgaccccg

SEQ ID NO: 701 - protein, 18 aa, Bacillus thuringiensis
  VARIANT 4, 7, 10: Xaa = any amino acid
DWTXWSXLVXAACSVELL

SEQ ID NO: 702 - DNA, 54 bp, Bacillus thuringiensis; identical to SEQ ID NO: 389
  misc_feature 10, 11, 12, 19, 20, 21, 28, 29, 30: n = A,T,C or G

SEQ ID NO: 703 - protein, 30 aa, Lactobacillus curvatus L442; identical to SEQ ID NO: 390

SEQ ID NO: 704 - DNA, 90 bp, Lactobacillus curvatus L442; identical to SEQ ID NO: 391

SEQ ID NO: 705 - protein, 43 aa, Carnobacterium divergens; identical to SEQ ID NO: 392

SEQ ID NO: 706 - DNA, 129 bp, Carnobacterium divergens; identical to SEQ ID NO: 393

SEQ ID NO: 707 - protein, 24 aa, Microbispora sp. (strain 107891)
VTSWSLCTPGCTSPGGGSNCSFCC

SEQ ID NO: 708 - DNA, 72 bp, Microbispora sp. (strain 107891)
gtgaccagctggagcctgtgcaccccgggctgcaccagcccgggcggcggcagcaactgcagcttttgctgc

SEQ ID NO: 709 - protein, 62 aa, Enterococcus sp.; identical to SEQ ID NO: 394

SEQ ID NO: 710 - DNA, 186 bp, Enterococcus sp.; identical to SEQ ID NO: 395

SEQ ID NO: 711 - protein, 39 aa, Enterococcus faecium; identical to SEQ ID NO: 396

SEQ ID NO: 712 - DNA, 117 bp, Enterococcus faecium; identical to SEQ ID NO: 397

SEQ ID NO: 713 - protein, 30 aa, Paenibacillus polymyxa; identical to SEQ ID NO: 398

SEQ ID NO: 714 - DNA, 90 bp, Paenibacillus polymyxa; identical to SEQ ID NO: 399

SEQ ID NO: 715 - protein, 31 aa, Staphylococcus epidermidis; identical to SEQ ID NO: 400

SEQ ID NO: 716 - DNA, 93 bp, Staphylococcus epidermidis; identical to SEQ ID NO: 401

SEQ ID NO: 717 - protein, 58 aa, Enterococcus faecium
MEKLTVKEMSQVVGGKYYGNGVSCNKKGCSVDWGKAIGIIGNNAAANLTTGGKAGWKG

SEQ ID NO: 718 - DNA, 178 bp, Enterococcus faecium
atggaaaaattaactgtgaaagaaatgtcgcaagtagttggcggaaagtactatggtaacggagtatcatgtaataaaaagggatgtagtgttgattggggaaaagctattggtattattggaaataatgctgctgctaatttaactactggcggaaaagcagggtggaaaggttaac

SEQ ID NO: 719 - protein, 39 aa, Paenibacillus polymyxa; identical to SEQ ID NO: 404

SEQ ID NO: 720 - DNA, 93 bp, Paenibacillus polymyxa; identical to SEQ ID NO: 401

SEQ ID NO: 721 - protein, 35 aa, Bacillus circulans; identical to SEQ ID NO: 406

SEQ ID NO: 722 - DNA, 105 bp, Bacillus circulans; identical to SEQ ID NO: 407

SEQ ID NO: 723 - protein, 30 aa, Paenibacillus polymyxa; identical to SEQ ID NO: 408

SEQ ID NO: 724 - DNA, 90 bp, Paenibacillus polymyxa; identical to SEQ ID NO: 409

SEQ ID NO: 725 - protein, 13 aa, Lactobacillus rhamnosus
AVPAVRKTNETLD

SEQ ID NO: 726 - DNA, 39 bp, Lactobacillus rhamnosus; identical to SEQ ID NO: 411

SEQ ID NO: 727 - protein, 60 aa, Bacillus licheniformis
MKNSAAREAFKGANHPAGMVSEEELKALVGGNDVNPETTPATTSSWTCITAGVTVSASLC

SEQ ID NO: 728 - DNA, 180 bp, Bacillus licheniformis
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgc

SEQ ID NO: 729 - protein, 689 aa, Pseudomonas aeruginosa; identical to SEQ ID NO: 276

SEQ ID NO: 730 - DNA, 2070 bp, Pseudomonas aeruginosa; identical to SEQ ID NO: 277

SEQ ID NO: 731 - protein, 36 aa, Lactobacillus plantarum; identical to SEQ ID NO: 414

SEQ ID NO: 732 - DNA, 108 bp, Lactobacillus plantarum; identical to SEQ ID NO: 415

SEQ ID NO: 733 - protein, 30 aa, Lactococcus lactis
MKTILRFVAGYDIASHKKKTGGYPWERGKA

SEQ ID NO: 734 - DNA, 93 bp, Lactococcus lactis
atgaaaacaatcctacgttttgttgctggctacgatattgctagtcataaaaagaaaactggcggctatccatgggaacgtggaaaagcttaa

SEQ ID NO: 735 - protein, 24 aa, Lactobacillus acidophilus
GNPKVAHCASQIGRSTAWGAVSGA

SEQ ID NO: 736 - DNA, 72 bp, Lactobacillus acidophilus; identical to SEQ ID NO: 417

SEQ ID NO: 737 - protein, 53 aa, Lactobacillus salivarius cp400
MFFNFMKKVDVKKNFGYKEVSRKDLAKVNGGKRKKHRCRVYNNGMPTGMYRWC

SEQ ID NO: 738 - DNA, 162 bp, Lactobacillus salivarius cp400
atgttttttaattttatgaaaaaagtagatgtgaagaagaattttggatataaagaagtttctagaaaagatctagctaaagtaaatggtggaaagagaaagaaacatcgttgcagagtttataataatggaatgcctacaggaatgtatcgttggtgctaa

SEQ ID NO: 739 - protein, 5 aa, Artificial Sequence
  Caspase 2 consensus sequence
DVADL

SEQ ID NO: 740 - protein, 5 aa, Artificial Sequence
  Caspase 2 consensus sequence
DVADI

SEQ ID NO: 741 - protein, 12 aa, Artificial Sequence
  Synthetic RAP-binding peptide, RBP
FHWWQTSPAHFS

SEQ ID NO: 742 - protein, 12 aa, Artificial Sequence
  Synthetic RAP-binding peptide, RBP
WPFAHWPWQYPR

SEQ ID NO: 743 - protein, 9 aa, Artificial Sequence
  Synthetic AgrC ligand
  VARIANT 5, 9: Thiolacton linkage between C5 and F9
GDSVCASYF

SEQ ID NO: 744 - protein, 7 aa, Artificial Sequence
  Synthetic AgrC ligand
  VARIANT 3, 7: Thiolacton linkage between C3 and F7
SVCASYF

SEQ ID NO: 745 - protein, 5 aa, Artificial Sequence
  Synthetic Cry1Aa ligand
SKADT

SEQ ID NO: 746 - protein, 5 aa, Artificial Sequence
  Synthetic Cry1Aa ligand
SKPAD

SEQ ID NO: 747 - protein, 11 aa, Artificial Sequence
  Synthetic Fsr ligand
  VARIANT 3, 11: Lacton linkage between S3 and A11
QNSAAAFAAWA

SEQ ID NO: 748 - protein, 11 aa, Artificial Sequence
  Synthetic Fsr ligand
  VARIANT 3, 11: Lacton linkage between S3 and A11
QNSAAAFGQWA

SEQ ID NO: 749 - protein, 7 aa, Artificial Sequence
  Synthetic AgrC1, AgrC2
  VARIANT 4, 7: Thiolacton linkage between C4 and M7)
YSTCFIM

SEQ ID NO: 750 - protein, 7 aa, Artificial Sequence
  Synthetic AgrC1, AgrC2
  VARIANT 3, 7: Thiolacton linkage between C3 and M7
STCAFIM

(19) United States
(12) Patent Application Publication
(10) Pub. No.: US 2023/0416797 A1
(43) Pub. Date: Dec. 28, 2023

(54) MODULATING ANTIMICROBIAL PEPTIDE HALF-LIFE

(71) Applicant: Syngulon SA., Seraing (BE)

(72) Inventor: Philippe Gabant, Ottignies Louvain-La-Neuve (BE)

(21) Appl. No.: 18/252,026

(22) PCT Filed: Nov. 5, 2021
(86) PCT No.: PCT/US2021/072262

§ 371 Date: May 5, 2023

Related U.S. Application Data
(60) Provisional application No. 63/112,073, filed on Nov. 10, 2020.

(51) Int. Cl. C12P 21/02; C07K 14/81

(52) U.S. Cl. CPC C12P 21/02; C07K 14/8121

(57) ABSTRACT

Methods of modulating the half-life of an antimicrobial peptide are described. The method may include administering an antimicrobial peptide to an environment where the antimicrobial peptide remains active until a specified endpoint, and on or after the specified endpoint, digesting the antimicrobial peptide with a protease to thereby inactivate the antimicrobial peptide and modulate its half-life. Compositions and kits for modulating the half-life of an antimicrobial peptide are also described.

### REFERENCE TO RELATED APPLICATIONS

This Application claims benefit of U.S. Provisional Application No. 63/112073, filed Nov. 10, 2020. The entirety of this related application is incorporated herein by reference.

### REFERENCE TO SEQUENCE LISTING, TABLE, OR COMPUTER PROGRAM LISTING

The present application is being filed along with a Sequence Listing in electronic format. The Sequence Listing is provided as a file entitled SYNG008WOSEQUENCE.TXT, created and last saved on Oct. 27, 2021, which is 402,803 bytes in size. The information in the electronic format of the Sequence Listing is incorporated herein by reference in its entirety.

### BACKGROUND

Regulating or inhibiting the growth of microbial organisms is important in various industrial and medical environments, such as fermenters, production facilities for food products, pharmaceuticals and cosmetic products, and medical devices and facilities. Antimicrobial compounds that inhibit growth of microbial organisms can be effective in controlling the growth of microbial organisms.

Antimicrobial peptides can regulate or inhibit the growth of microbial organisms.

### Field

Embodiments of the present disclosure relate to the use of antimicrobial peptides in an environment and the modulation of the half-life of the antimicrobial peptides with proteases.

### SUMMARY

Described herein are methods of modulating a half-life of an antimicrobial peptide (also referred to as a “modulation method”). Embodiments of the modulation method may include administering an antimicrobial peptide to an environment, in which the antimicrobial peptide remains active in the environment until a specified endpoint; on or after the specified endpoint, digesting the antimicrobial peptide with a protease, thereby inactivating the antimicrobial peptide, thus modulating the half-life of the antimicrobial peptide. In the modulation method of some embodiments, the method can include identifying the environment as permissive for the growth of microbial organisms capable of developing resistance to the antimicrobial peptide. The antimicrobial peptide can be digested with the protease before any microbial organisms develop resistance to the anti-microbial peptide. In the modulation method of some embodiments, the antimicrobial peptide is digested within the environment. In the modulation method of some embodiments, the antimicrobial peptide inhibits the growth or reproduction of an undesired microbial organism (which may also be referred to herein as an “undesirable microbial organism”). In the modulation method of some embodiments, the antimicrobial peptide is digested with the protease before the undesired microbial organism or a clone thereof develops resistance to the antimicrobial peptide. For example, the undesired microbial organism can be selected from the group consisting of a pathogen, a contaminant, and an industrial microbe that has lost an industrial characteristic. In the modulation method of some embodiments, the method includes adding a serpin to the environment. The serpin can inhibit the protease. In the modulation method of some embodiments, the antimicrobial peptide is digested by the protease in the environment. In the modulation method of some embodiments, the antimicrobial peptide is digested by the protease outside of the environment.

In the modulation method of some embodiments, the protease is disposed on a substrate, and digesting the antimicrobial peptide comprises contacting the antimicrobial peptide with the substrate. For example, the protease may be immobilized on the substrate, for example via a covalent or non-covalent bond. By way of example, the substrate may comprise a particle, a bead, a capsule, or a matrix such as a gel matrix. In the modulation method of some embodiments, the environment comprises an organ of a subject, a microbiome in vivo, a food product or portion thereof, a medical device, an industrial feedstock, or a pharmaceutical or cosmetic manufacturing environment. For example, the environment may comprise the organ of the subject or the microbiome in vivo. Optionally, the method may further comprise an ex vivo selection for the antimicrobial peptide prior to the administering.

In any of modulation methods described herein, and in accordance with some embodiments, the antimicrobial peptide may be administered to a subject, in which the environment comprises a pathogenic microbial organism, and in which the specified endpoint comprises inhibiting the growth or reproduction of the pathogenic microbial organism. In any of the modulation methods described herein, and in accordance with some embodiments, the specified endpoint may be a specified period of time, a decrease in a quantity of a microbial organism targeted by the antimicrobial peptide to a level below a threshold, a decrease in an activity level of a microbial organism targeted by the antimicrobial peptide to a level below a threshold, or a change in the environment. In any of the modulation methods described herein, and in accordance with some embodiments, the environment may comprise a first genetically engineered microbial organism comprising a nucleic acid encoding the antimicrobial peptide, and administering the antimicrobial peptide may comprise the genetically engineered microbial organism expressing and secreting the antimicrobial peptide into the environment. For example, the first genetically engineered microbial organism can comprise a nucleic acid encoding the protease, and is configured to induce transcription of the nucleic acid encoding the protease, translation of a transcript of the nucleic acid encoding the protease, or secretion of the encoded protease upon the specified endpoint.

In any of the modulation methods described herein, and in accordance with some embodiments, the environment may comprise a second genetically engineered microbial organism comprising a nucleic acid encoding the protease, and configured to induce transcription of the nucleic acid encoding the protease, translation of a transcript of the nucleic acid encoding the protease, or secretion of the encoded protease upon the specified endpoint. In the modulation method of some embodiments, the antimicrobial peptide is selected from the group consisting of a bacteriocin, an antibacterial peptide, an antiviral peptide, an anti-HIV peptide, an antifungal peptide, an antiparasitic peptide, and an anticancer peptide. In the modulation method of some embodiments, the antimicrobial peptide is administered in a cocktail further comprising a first additional antimicrobial peptide different from the antimicrobial peptide, in which the first additional antimicrobial peptide is not digested. In some embodiments, the cocktail further comprises a second additional antimicrobial peptide that is different from the antimicrobial peptide, in which the second additional antimicrobial peptide is digested by a second protease that is different from the protease. In some embodiments, at least one of the additional antimicrobial peptides is digested by the protease.

In any of modulation methods described herein, and in accordance with some embodiments, the method may further include selecting the antimicrobial peptide to be trypsin-sensitive. Prior to the specified endpoint, the antimicrobial peptide may inhibit the growth or reproduction of microbial organisms that cause food spoilage. The specified endpoint can be consumption of the food product, and the protease can comprise trypsin. That is, in some embodiments, an antimicrobial peptide that inhibits food spoilage may be present in a food product, and upon consumption by a subject, may be degraded by trypsin in the subject's gastrointestinal tract (as such, the specified endpoint can be consumption of the food product).

In any of the modulation methods described herein, and in accordance with some embodiments, digesting antimicrobial peptide decreases the half-life of the antimicrobial peptide by at least 50%, for example at least 60%, 70%, 80%, 90%, or 95%. In any of modulation methods described herein, and in accordance with some embodiments, digesting the antimicrobial peptide comprises digesting the antimicrobial peptide with two or more different proteases. In any of the modulation methods described herein, and in accordance with some embodiments, the protease comprises a genetically engineered or synthetic protease. By way of example, the synthetic protease can be selected for a desired protease activity in vitro or in vivo. In any of the modulation methods described herein, and in accordance with some embodiments, the method includes producing a product from the environment, in which the antimicrobial peptide is digested prior to producing the product, so that the product does not comprise the antimicrobial peptide. For example, the product can be selected from the group consisting of an industrial product, a pharmaceutical product, a cosmetic product, and a food product.

Also described are compositions comprising a protease, an antimicrobial peptide comprising a cleavage site that is specifically digested by the protease; and a microbial organism capable of developing resistance to the antimicrobial peptide.

Also described are kits comprising an antimicrobial peptide selected to target a microbial organism capable of developing resistance to the antimicrobial peptide; and a protease capable of digesting the antimicrobial peptide. In the kit of some embodiments, the antimicrobial peptide and the protease are in separate compositions. In the kit of some embodiments, the antimicrobial peptide and the protease are in the same composition, and the protease is disposed to not digest the antimicrobial peptide prior to a specified endpoint. For example, the protease can be disposed with a serpin configured to antagonize the protease. In the kit of some embodiments, the protease is physically separated from the antimicrobial peptide. In any of the kits describe herein, and in accordance with some embodiments, the antimicrobial peptide and/or protease are formulated for in vivo administration to a human subject. In any of the kits describe herein, and in accordance with some embodiments, the antimicrobial peptide is selected from the group consisting of a bacteriocin, an antibacterial peptide, an antiviral peptide, an anti-HIV peptide, an antifungal peptide, an antiparasitic peptide, and an anticancer peptide. In any of the kits described herein, and in accordance with some embodiments, the kit further includes a cocktail of antimicrobial peptides comprising the antimicrobial peptide, wherein the protease is incapable of digesting at least some antimicrobial peptides of the cocktail. In any of the kits described herein, and in accordance with some embodiments, the kit further includes a serpin selected to inhibit the protease.

### BRIEF DESCRIPTION OF THE DRAWINGS

FIG. 1 is a flow diagram of a method of modulating the half-life of an antimicrobial peptide, according to some embodiments of the present disclosure.

FIG. 2 is a schematic diagram depicting a method for modulating the half-life of an antimicrobial peptide, according to some embodiments of the present disclosure.

FIG. 3 is a schematic diagram depicting a method for modulating the half-life of an antimicrobial peptide, according to some embodiments of the present disclosure.

### DETAILED DESCRIPTION

Provided herein are methods of modulating the half-life of an antimicrobial peptide in an environment (which may be referred to as “modulation method(s)” herein). The modulation methods can include administering an antimicrobial peptide to an environment, and subsequently inactivating the antimicrobial peptide, e.g., by digestion with a protease. For example, the antimicrobial peptide can be inactivated at or after a specified endpoint (for example, once undesired microbial organisms are at or below a certain level), at which point the antimicrobial peptide is not needed or desired. It is contemplated that inactivating the antimicrobial protein can avoid or decrease the likelihood of microbial organisms developing resistance to the antimicrobial peptide.

Digesting the antimicrobial peptide by a protease may modulate the half-life of the antimicrobial peptide. “Half-life” as used herein has its customary and ordinary meanings as understood by one of skill in the art in view of this disclosure. The half-life of the antimicrobial peptide may be based on the activity level (e.g., level of inhibition of the growth of a target microbial organism by the antimicrobial peptide) or the amount of intact (e.g., full-length) antimicrobial peptide in the environment. In some embodiments, the half-life of the antimicrobial peptide is the time it takes for the activity level of the antimicrobial peptide to be reduced by 50% (e.g., the time for a first activity level of the antimicrobial peptide to be reduced to 50% of the first activity level). For example, the activity level can refer to the level of inhibition of the growth of a target microbial organism when the antimicrobial peptide is administered to the environment. In some embodiments, the half-life of the antimicrobial peptide is the time it takes for the amount of intact antimicrobial peptide to be reduced by 50%. In some embodiments, digesting the antimicrobial peptide with the protease reduces the half-life of the antimicrobial peptide by 10% or more, e.g., 20% or more, 30% or more, 40% or more, 50% or more, 60% or more, 70% or more, 80% or more, 90% or more, or about 100%, including ranges between any two of the listed values, for example 10%-30%, 10%-50%, 10%-90%, 20%-50%, 20%-90%, 30%-50%, 30%-90%, or 50%-90%.

Targets of the antimicrobial peptide may include one or more microbial organisms (which may be referred to as “target microbial organisms” or “target microorganisms”). The target microbial organism may be an undesired microbial organism, such as, but not limited to, a pathogen, a contaminating microbe, or a microbe used in industrial production but has lost its industrial characteristic. In some embodiments, the environment may allow growth of microbial organisms (e.g., contaminating or otherwise undesired microbial organisms) that can develop resistance to the antimicrobial peptide. By inactivating the antimicrobial peptide, e.g., through digestion with a protease, the microbial organism may be inhibited or prevented from developing resistance to the antimicrobial peptide. In some embodiments, it may be desirable to restrict the activity of the antimicrobial peptide to a certain environment such that the antimicrobial peptide is not present and active outside of that environment, e.g., to inhibit or prevent resistance to the antimicrobial peptide from developing outside of the environment.

In the modulation methods of some embodiments, an antimicrobial peptide may be administered to any suitable environment in which it is desirable to modulate the half-life of the antimicrobial peptide, e.g., where it may be desirable to inhibit or prevent a microbial organism from developing resistance to the antimicrobial peptide. Suitable environments include, without limitation, an organ of a subject (e.g., a mammalian subject, including a human subject), a microbiome in vivo, a food product or portion thereof, a medical device, an industrial feedstock, or a pharmaceutical or cosmetic manufacturing environment (such as a bioreactor), or a combination of two or more of the listed items. Where the environment is used to produce a product, e.g., a manufacturing environment for a pharmaceutical, cosmetic or food product, it may be desirable to inactivate the antimicrobial peptide before the product is made and/or leaves the manufacturing environment, such that the product itself does not contain active antimicrobial peptides outside of the manufacturing environment. Thus, in some embodiments, the antimicrobial peptide is digested within the environment.

Also described are compositions that comprise, consist of, or consist essentially of a protease, an antimicrobial peptide that includes a cleavage site specifically cleaved by the protease, and a microbial organism capable of developing resistance to the antimicrobial peptide. In some embodiments, kits comprise, consist of, or consist essentially of an antimicrobial peptide (or nucleic acid encoding an antimicrobial peptide) targeting a microbial organism capable of developing resistance to the antimicrobial peptide, and a protease (or nucleic acid encoding a protease) capable of digesting the antimicrobial peptide, are also provided.

### Microbial Organisms

As used herein, “microbial organism,” “microorganism,” and variations of these root terms (such as pluralizations and the like), have their customary and ordinary meanings as understood by one of skill in the art in view of this disclosure. They encompass any naturally-occurring species or fully synthetic prokaryotic or eukaryotic unicellular organism, as well as Archae species. Thus, this term can refer to cells of bacterial species, fungal species, and algae. “Microbial organism” and “microorganism” may be used interchangeably herein, as may corresponding variations of these root terms.

Suitable microorganisms that can be used in accordance with embodiments herein, or whose growth may be inhibited with an antimicrobial peptide, include but are not limited to bacteria, yeast, and algae, for example photosynthetic microalgae. Furthermore, fully synthetic microorganism genomes can be synthesized and transplanted into single microbial cells, to produce synthetic microorganisms capable of continuous self-replication (see Gibson et al. (2010), “Creation of a Bacterial Cell Controlled by a Chemically Synthesized Genome,” Science 329: 52-56, hereby incorporated by reference in its entirety). As such, in some embodiments, the microorganism is fully synthetic. A desired combination of genetic elements, including elements that regulate gene expression, and elements encoding gene products (for example antimicrobial peptides, immunity modulators, protease, poison, antidote, and industrially useful molecules) can be assembled on a desired chassis into a partially or fully synthetic microorganism. Description of genetically engineered microbial organisms for industrial applications can also be found in Wright, et al. (2013) “Building-in biosafety for synthetic biology” Microbiology 159: 1221-1235.

A variety of bacterial species and strains can be used, or may be targeted by an antimicrobial peptide, in accordance with embodiments herein, and genetically modified variants, or synthetic bacteria based on a “chassis” of a known species can be provided. Exemplary bacteria with industrially applicable characteristics, which can be used in accordance with embodiments herein include, but are not limited to, Bacillus species (for example Bacillus coagulans, Bacillus subtilis, and Bacillus licheniformis), Paenibacillus species, Streptomyces species, Micrococcus species, Corynebacterium species, Acetobacter species, Cyanobacteria species, Salmonella species, Rhodococcus species, Pseudomonas species, Lactobacillus species, Enterococcus species, Bifidobacterium species, Bacteroides species, Alcaligenes species, Klebsiella species, Paenibacillus species, Arthrobacter species, Corynebacterium species, Brevibacterium species, Thermus aquaticus, Pseudomonas stutzeri, Clostridium thermocellus, and Escherichia coli.

A variety of yeast species and strains can be used, or may be targeted by an antimicrobial peptide, in accordance with embodiments herein, and genetically modified variants, or synthetic yeast based on a “chassis” of a known species can be provided. Exemplary yeast with industrially applicable characteristics, which can be used in accordance with embodiments herein include, but are not limited to Saccharomyces species (for example, Saccharomyces cerevisiae, Saccharomyces bayanus, Saccharomyces boulardii), Candida species (for example, Candida utilis, Candida krusei), Schizosaccharomyces species (for example Schizosaccharomyces pombe, Schizosaccharomyces japonicas), Pichia or Hansenula species (for example, Pichia pastoris or Hansenula polymorpha) species, and Brettanomyces species (for example, Brettanomyces claussenii).

A variety of algae species and strains can be used, or may be targeted by an antimicrobial peptide, in accordance with embodiments herein, and genetically modified variants, or synthetic algae based on a “chassis” of a known species can be created. In some embodiments, the algae comprises photosynthetic microalgae. Exemplary algae species that can be useful for biofuels, and can be used in accordance with embodiments herein, include Botryococcus braunii, Chlorella species, Dunaliella tertiolecta, Gracilaria species, Pleurochrysis carterae, and Sargassum species. Additionally, many algae species can be useful for food products, fertilizer products, waste neutralization, environmental remediation, and carbohydrate manufacturing (for example, biofuels).

A desired microbial organism may be beneficial to the environment for any suitable purpose. In some embodiments, the desired microbial organism provides a benefit to, e.g., the growth and/or maintenance of a microbial community in the environment, the larger environment to which the microbial community belongs, the purpose for which the microbial organism is used, etc. In some embodiments, an environment of the present disclosure is contaminated by an undesired microbial organism. The undesired microbial organism may be undesirable for any relevant reason. In some embodiments, the undesired microbial organisms is a pathogen. For example, the undesired microbial organism may be pathogenic to a host organism (e.g., mammal) of the microbial community (e.g., a host comprising a microbiome). In some embodiments, the undesired microbial organism may be pathogenic to organisms that grow in or around the microbial community. In some embodiments, the undesired microbial organism may be pathogenic to organisms that grow in or around the microbial community. In some embodiments, the undesired microbial organism is a microbial organism that competes with and/or interferes with the growth of the desired microbial organism in the environment. In some embodiments, the undesired microbial organism is selected from the group consisting of a pathogen, a contaminant, and a microbial organism that competes with and/or interferes with the growth of the desired microbial organism in the microbial community. In some embodiments, the undesired microbial organism is an industrial microbe that has lost its industrial characteristic. For example, the industrial microbe is a microbial organism suitable for producing an industrial product (e.g., a food product, pharmaceutical or cosmetic product, etc.) in the environment. In some embodiments, the environment includes a precursor ingredient that is converted to an industrial product by the industrial microbe, and the industrial characteristic is lost when the precursor ingredient is depleted from the environment. In some embodiments, the industrial microbe is genetically engineered to produce a product, and the industrial characteristic is lost when microbial organism loses some or all of the nucleic acid with which it was genetically engineered. In some embodiments, the undesired microbial organism interferes with the production of a product, such as a food, cosmetic, pharmaceutical, or industrial product, for example by contaminating the product or a precursor thereof, or by consuming, damaging, or destroying the product or a precursor thereof.

In some embodiments, a microbial organism (e.g., an undesired microbial organism) is capable of developing resistance to an antimicrobial peptide. “Developing resistance” as used herein with reference to an antimicrobial peptide, has its ordinary and customary meaning as would be understood by one of ordinary skill in the art in view of this disclosure. It refers to the microbial organism becoming less susceptible to the growth-inhibitory and/or killing effects of the antimicrobial peptide when grown in the presence of the antimicrobial peptide. For example, the LD₅₀ of the antimicrobial peptide may increase as the resistance develops. The resistance may develop over one or more generations of growth by the microbial organism. Thus, resistance to the antimicrobial peptide may be acquired in the microbial organism, including any clone or descendent thereof.

Development of resistance may be determined by any suitable measure. By way of example, development of resistance can be determined by comparing the quantity or rate of growth of the microbial organism in the presence of an effective amount of the antimicrobial peptide with that of a reference. For example, the reference can comprise a microbial organism of the same type or strain of microbial organism in the presence of the antimicrobial peptide, in which the reference microbial organism is naïve to the antimicrobial peptide (that is, the reference microbial organism that has not been grown in the presence of an effective amount of the antimicrobial peptide). In some embodiments, a microbial organism has not developed resistance to the antimicrobial peptide if the antimicrobial peptide has an LD₅₀ for that microbial organism that is no more than 100%, 110%, 120%, 130%, 150%, or 200%, of the LD₅₀ for a reference microbial organism of the same strain or species that is naïve to the antimicrobial peptide. In some embodiments, the microbial organism has not developed resistance to the antimicrobial peptide if the growth rate in the presence of an effective amount of the antimicrobial peptide is 30% or more, e.g., 40% or more, 50% or more, 60% or more, 70% or more, 80% or more, 90% or more, 95% or more, or 100% or more of the growth rate of a reference.

In some embodiments, development of resistance is determined by comparing the inhibitory effect of an effective amount of the antimicrobial peptide on the growth rate of the microbial organism with a reference inhibitory effect, such as an effect of the antimicrobial peptide on the growth rate of a reference microbial organism naïve to the antimicrobial peptide, or the effect of a different antimicrobial compound on the growth rate of the microbial organism. In some embodiments, the reference inhibitory effect on growth is the reduction in the rate of growth induced by an effective amount of antimicrobial peptide in the same type or strain of microbial organism but that is naïve to (e.g., has not been previously grown in the presence of) the antimicrobial peptide. In some embodiments, the microbial organism has not developed resistance to the antimicrobial peptide if the reduction in the rate of growth induced by an effective amount of antimicrobial peptide is 30% or more, e.g., 40% or more, 50% or more, 60% or more, 70% or more, 80% or more, 90% or more, 95% or more, or 100% or more of the reference inhibitory effect on growth.

Without being limited by theory, the ability of the microbial organism of some embodiments (e.g., an undesired microbial organism) to develop resistance to an antimicrobial peptide may depend on the environment and/or the properties of the antimicrobial peptide present in the environment, as well as the microbial organism itself. In some embodiments, the environment is permissive for the growth of microbial organisms that are capable of developing resistance to the antimicrobial peptide. Thus, the microbial organism may be of a type that has a tendency, predisposition, or capability to develop resistance if grown in the presence of the antimicrobial peptide in the environment. In some embodiments, an undesired microbial organism is capable of developing resistance if grown in the presence of the antimicrobial peptide in the environment. In some embodiments, the microbial organism targeted by the administered antimicrobial peptide has a mutation rate that is sufficiently high such that the microbial organism can gain one or more mutations that confer resistance to the antimicrobial peptide during growth in the environment, for example a mutation in a coding sequence for a channel or transporter that removes the antimicrobial peptide, a mutation in a receptor or protein that interacts with the antimicrobial peptide, or a mutation in a protease that degrades the antimicrobial peptide. In some embodiments, the microbial organism targeted by the administered antimicrobial peptide is partially resistant to the antimicrobial peptide such that resistant variants of the microbial organism preferentially survive in the environment. It will be appreciated that antimicrobial resistance may be acquired gradually, through multiple round of selection across multiple generations of microbial organism.

### Antimicrobial Peptides

As used herein “antimicrobial peptide” (including variations of these root terms such as “antimicrobial peptide”) has its customary and ordinary meaning as would be understood by one of ordinary skill in the art in view of this disclosure. It refers to a class of peptides that kill or arrest the growth of microbial organisms. While antimicrobial peptides have classically been referred to as a class of invertebrate and vertebrate gene products that target microbial organisms, bacteriocins have classically been referred to a class of microbial gene products that target microbial organisms. However, for conciseness “antimicrobial peptide” as used herein broadly encompasses classical antimicrobial peptides (e.g., that confer innate immune activity against microbial organisms) as well as bacteriocins. Thus, suitable antimicrobial peptides include polypeptides derived from any source (e.g., derived from prokaryotes, or eukaryotes, such as mammals, fungi, plants, etc., or partially or fully synthetic) that reduce or inhibit growth of, or kill microbial organisms. In some embodiments, antimicrobial peptides comprise, consist essentially of, or consist of peptides of the innate immune systems of invertebrates and vertebrates. Thus, in some embodiments, antimicrobial peptides include a class of invertebrate and vertebrate gene products that target microbial organisms.

Examples of suitable antimicrobial peptides can be found, for example, at The Antimicrobial Peptide Database accessible on the world wide web at aps.unmc.edu/AP/, which is incorporated herein by reference in its entirety. Over 1000 antimicrobial peptides and variants thereof have been identified and cataloged. The Antimicrobial Peptide Database is described in Wang et al. (2016), Nucleic Acids Res. 44(Database issue): D1087-D1093, which is incorporated herein by reference in its entirety. Examples of antimicrobial peptides suitable for embodiments herein (such as in production methods and/or microbial communities) include bacteriocins, antibacterial, antiviral, anti-HIV, antifungal, antiparasitic and anticancer peptides, such as dermaseptins (e.g., Dermaseptin-B2), abaecin, Ct-AMP1, andropin, apidaecin, cecropin, ceratotoxin, dermacidin, Maximin H5, moricin, melittin, magainin, bombinin, brevinin, esculentin, buforin, CAP18, LL37, protegrin, prophenin, indolicidin, tachyplesins, defensin, drosomycin, aurein 1.1, Lactoferricin B, and Heliomicin, or a combination of two or more of any of the listed items. In some embodiments, the antimicrobial peptide comprises a bacteriocin, dermaseptins (e.g., Dermaseptin-B2), abaecin, Ct-AMP1, andropin, apidaecin, cecropin, ceratotoxin, dermacidin, Maximin H5, moricin, melittin, magainin, bombinin, brevinin, esculentin, buforin, CAP18, LL37, protegrin, prophenin, indolicidin, tachyplesins, defensin, drosomycin, aurein 1.1, Lactoferricin B, and Heliomicin, or a combination of two or more of any of the listed items. Antimicrobial peptides of the present disclosure in some embodiments include naturally-occurring antimicrobial peptides or mutants or variants thereof, or a nucleic acid encoding the same. In some embodiments, antimicrobial peptides of the present disclosure include non-naturally occurring antimicrobial peptides (such as partially or fully synthetic antimicrobial peptides or variant antimicrobial peptides), or nucleic acids encoding the same. In some embodiments, antimicrobial peptides of the present disclosure are non-naturally occurring peptide sequences, or nucleic acids encoding the same. In some embodiments, an antimicrobial peptide has at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, identity to a reference antimicrobial peptide (for example Dermaseptin-B2, Abaecin, Ct-AMPL Andropin, Aurein 1.1, Lactoferricin B, or Heliomicin, or any of SEQ ID NOs: 4-450 (even numbers) and 699-737 (odd numbers), including ranges between any two of the listed values, for example 70%-99%, 75%-99%, 80%-99%, 85%-99%, 90%-99%, 95%-99%, 97%-99%, 70%-95%, 75%-95%, 80%-95%, 85%-95%, 90%-95%, 70%-90%, 75%-90%, 80%-90%, and 85%-90%. Such an antimicrobial peptide may be referred to as “variant” antimicrobial peptide. Percent identity may be determined using the BLAST software (Altschul. S. F., et al. (1990) “Basic local alignment search tool.” J. Mol. Biol. 215:403-410, accessible on the world wide web at blast.ncbi.nlm.nih.gov) with the default parameters.

In some embodiments, an antimicrobial peptide of the present disclosure comprises, consists essentially of, or consists of a bacteriocin. As used herein, “bacteriocin,” and variations of this root term, has its customary and ordinary meaning as understood by one of skill in the art in view of this disclosure. It refers to a polypeptide that is secreted by a host cell and can neutralize at least one cell other than the individual host cell in which the polypeptide is made, including cells clonally related to the host cell and other microbial cells. A cell that expresses a particular “immunity modulator” (discussed in more detail herein) is immune to the neutralizing effects of a particular bacteriocin or group of bacteriocins. As such, bacteriocins can neutralize a cell producing the bacteriocin and/or other microbial cells, so long as these cells do not produce an appropriate immunity modulator. As such, a host cell can exert cytotoxic or growth-inhibiting effects on a plurality of other microbial organisms by secreting bacteriocins. Example bacteriocins are set forth in SEQ ID NOS: 4-450 (even numbers) and 699-737 (odd numbers). Example nucleic acids encoding these bacteriocins are provided as SEQ ID NOs: 5-451 (odd numbers) and 700-738 (even numbers). Detailed descriptions of bacteriocins, including methods and compositions for using bacteriocins to control the growth of microbial cells can be found, for example, in U.S. Pat. No. 9,333,227, which is hereby incorporated by reference in its entirety. “Bacteriocin” is not limited by the origin of the polypeptide, and by way of example is contemplated to encompass any bacteriocin, such as naturally-occurring bacteriocins, synthetic bacteriocins, and variants and combinations thereof. Examples of suitable bacteriocins are described in detail herein.

While many of the bacteriocins are naturally-occurring (for example, naturally occurring bacteriocins set forth in SEQ ID NOS: 4-450 (even numbers) and 699-737 (odd numbers)), the skilled artisan will appreciate that in some embodiments of the methods, systems and kits described herein, a bacteriocin comprises a naturally-occurring bacteriocin other than the bacteriocins and encoding nucleotide sequences of SEQ ID SEQ ID NOS: 4-450 (even numbers) and 699-737 (odd numbers), or a non-naturally-occurring bacteriocin or a synthetic bacteriocin (such as an engineered bacteriocin), or a variant thereof (which can also be a kind of engineered bacteriocin of some embodiments). In some embodiments, an engineered bacteriocin has enhanced or decreased levels of cytotoxic or growth inhibition activity on the same or a different microorganism or species of microorganism relative to a wild-type bacteriocin. In some embodiments, the antimicrobial peptide (or bacteriocin) does not comprise a lantibiotic.

Several motifs have been recognized as characteristic of bacteriocins. For example, the motif YGXGV (SEQ ID NO: 2), wherein X is any amino acid residue, is an N-terminal consensus sequence characteristic of a class Ha bacteriocin. Accordingly, in some embodiments, a candidate (or variant) bacteriocin (e.g., an engineered bacteriocin) comprises an N-terminal sequence with at least about 50% identity to SEQ ID NO: 2), or a variant thereof. In some embodiments, a candidate (or variant) bacteriocin (e.g., an engineered bacteriocin) comprises a N-terminal sequence comprising SEQ ID NO: 2). Additionally, some class IIb bacteriocins comprise a GxxxG motif. Without being limited by any particular theory, it is believed that the GxxxG motif can mediate association between helical proteins in the cell membrane, for example to facilitate bacteriocin-mediated neutralization through cell membrane interactions. As such, in some embodiments, the bacteriocin (e.g., the engineered bacteriocin) comprises a motif that facilitates interactions with the cell membrane. In some embodiments, the bacteriocin comprises a GxxxG motif. Optionally, the bacteriocin comprising a GxxxG motif can comprise a helical structure. In addition to structures described herein, “bacteriocin” as used herein also encompasses structures that have substantially the same effect on microbial cells as any of the bacteriocins explicitly provided herein.

A number of bacteriocins have been identified and characterized. Without being limited by theory, exemplary bacteriocins can be classified as “class I” bacteriocins, which typically undergo post-translational modification, and “class II” bacteriocins, which are typically unmodified. Additional information on classifying bacteriocins can be found in Cotter, P. D. et al. “Bacteriocins—a viable alternative to antibiotics” Nature Reviews Microbiology (2013) 11: 95-105, incorporated by reference in its entirety herein.

A number of bacteriocins can be used as antimicrobial peptides in accordance with modulation methods, kits, and compositions of embodiments herein. Example bacteriocins are set forth in SEQ ID NOS: 4-450 (even numbers) and 699-737 (odd numbers). Example nucleic acids encoding these bacteriocins are provided as SEQ ID NOs: 5-451 (odd numbers) and 700-738 (even numbers). Detailed descriptions of bacteriocins and some polynucleotide sequences that encode bacteriocins, including methods and compositions for using bacteriocins to control the growth of microbial cells can be found, for example, in U.S. Pat. No. 9,333,227, which is incorporated by reference in its entirety herein. Some examples of suitable bacteriocins are taught in Table 1.2 of U.S. Pat. No. 9,333,227, which is incorporated by reference in its entirety herein. “Bacteriocin” is not limited by the origin of the polypeptide, and by way of example is contemplated to encompass any bacteriocin, such as naturally-occurring bacteriocins, synthetic bacteriocins, and variants and combinations thereof. Examples of suitable bacteriocins are described in detail herein.

Some antimicrobial peptides (such as bacteriocins) have cytotoxic activity (e.g. “bacteriocide” effects), and thus can kill microbial organisms, for example bacteria, yeast, algae, synthetic microorganisms, and the like. Some antimicrobial peptides (such as bacteriocins) can inhibit the reproduction of microbial organisms (e.g. “bacteriostatic” effects), for example bacteria, yeast, algae, synthetic microorganisms, and the like, for example by arresting the cell cycle. Without being limited by theory, bacteriocins can effect neutralization of a target microbial cell in a variety of ways. For example, a bacteriocin can permeabilize a cell wall, thus depolarizing the cell wall and interfering with respiration.

“Antibiotic,” and variations of this root term, has its customary and ordinary meaning as understood by one of skill in the art in view of this disclosure. It refers to a metabolite, or an intermediate of a metabolic pathway which can kill or arrest the growth of at least one microbial cell. Some antibiotics can be produced by microbial cells, for example bacteria. Some antibiotics can be synthesized chemically. It is understood that bacteriocins are distinct from antibiotics, at least in that bacteriocins refer to gene products (which, in some embodiments, undergo additional post-translational processing) or synthetic analogs of the same, while antibiotics refer to intermediates or products of metabolic pathways or synthetic analogs of the same.

In some embodiments, an antimicrobial peptide (such as a bacteriocin) comprises a polypeptide that has undergone post-translational modifications, for example cleavage, or the addition of one or more functional groups.

In some embodiments, a fusion polypeptide comprising two or more antimicrobial peptides (such as bacteriocins) or portions thereof has a neutralizing activity against a broader range of microbial organisms than either individual antimicrobial peptide of the two or more antimicrobial peptides or portions thereof. For example, it has been shown that a hybrid antimicrobial peptide displays antimicrobial activity against pathogenic Gram-positive and Gram-negative bacteria (Acuña et al. (2012), FEBS Open Bio, 2: 12-19). It is noted that that Ent35-MccV fusion bacteriocin comprises, from N-terminus to C-terminus, an N-terminal glycine, Enterocin CRL35, a linker comprising three glycines, and a C-terminal Microcin V.

It is contemplated herein that an antimicrobial peptide (such as a bacteriocin) can comprise a fusion of two or more polypeptides, for example two or more polypeptides having antimicrobial (such as bacteriocin) activity. In some embodiments an antimicrobial peptide or a candidate antimicrobial peptide comprises a chimeric protein. In some embodiments, a variant antimicrobial peptide (such as a bacteriocin) or an engineered antimicrobial peptide (such as an engineered bacteriocin) comprises a fusion polypeptide comprising two or more antimicrobial peptides (such as bacteriocins). In some embodiments, a variant antimicrobial peptide (such as a bacteriocin) or an engineered antimicrobial peptide (such as a bacteriocin) comprises a chimeric protein comprising two or more antimicrobial peptides (such as bacteriocins), or fragments thereof. In some embodiments, the two or more antimicrobial peptides of the fusion comprise polypeptides of SEQ ID NOS: 4-450 (even numbers) and 699-737 (odd numbers), and or encoded by nucleic acids of SEQ ID NOs: 5-451 (odd numbers) and 700-738 (even numbers), or variants or modifications thereof. In some embodiments, the fusion polypeptide has a broader spectrum of activity than either individual antimicrobial peptide, for example having neutralizing activity against more microbial organisms, neutralizing activity under a broader range of environmental conditions, and/or a higher efficiency of neutralization activity. In some embodiments, the fusion polypeptide comprises two, three, four, five, six, seven, eight, nine, or ten antimicrobial peptides. In some embodiments, two or more antimicrobial peptide polypeptides are fused to each other via a covalent bond, for example a peptide linkage. In some embodiments, a linker is positioned between the two individual antimicrobial polypeptides of the fusion polypeptide. In some embodiments, the linker comprises one or glycines, for example about 1, 2, 3, 4, 5, 6, 7, 8, 9, 10, 11, 12, 13, 14, 15, 16, 17, 18, 19, or 20 glycines. In some embodiments, the linker is cleaved within the cell to produce the individual antimicrobial peptides (such as bacteriocins) included in the fusion protein. In some embodiments, a variant antimicrobial peptide (such as a variant bacteriocin) or engineered antimicrobial peptide (such as an engineered bacteriocin) as provided herein comprises a modification to provide a desired spectrum of activity relative to the unmodified or candidate antimicrobial peptide (e.g., bacteriocin). For example, the variant antimicrobial peptide (e.g., bacteriocin) or engineered antimicrobial peptide (e.g., bacteriocin) may have enhanced or decreased activity against the same organisms as the unmodified or candidate antimicrobial peptide (e.g., bacteriocin). Alternatively, the modified antimicrobial peptide (e.g., bacteriocin) may have enhanced activity against an organism against which the unmodified or candidate antimicrobial peptide (e.g., bacteriocin) has less activity or no activity.

In some embodiments, a particular neutralizing activity or range of activities for the antimicrobial peptide (e.g. cytotoxicity or arrest of microbial reproduction) is selected based on the type of antimicrobial regulation that is desired and the particular taxonomic category, species, or strain of microbial organisms being targeted. As such, in some embodiments, particular antimicrobial peptides or combinations of antimicrobial peptides are selected. For example, in some embodiments, a particular antimicrobial peptide is administered to the environment based on the undesired microbial organisms being regulated. In some embodiments, desired microbial organisms are engineered to express particular antimicrobial peptides based on the undesired microbial organisms being regulated. In some embodiments, for example if contaminating undesired microbial organisms are to be killed, at least one cytotoxic antimicrobial peptide (such as a cytotoxic bacteriocin) is provided (e.g., administered to the environment, secreted by a microbial organism, etc.). In some embodiments, a bacteriocin or combination of bacteriocins which is effective against contaminants which commonly occur in a particular culture, microbiome, a particular geographic location, or a particular type of culture grown in a particular geographic location or industrial culture are selected. In some embodiments, for example embodiments in which regulation of microbial cell ratios is desired, an antimicrobial peptide that inhibits microbial reproduction is provided. Without being limited by theory, many bacteriocins can have neutralizing activity against microbial organisms that typically occupy the same ecological niche as the species that produces the bacteriocin. As such, in some embodiments, when a particular spectrum of antimicrobial peptides activity is desired, a bacteriocin is selected from a host species that occupies the same (or similar) ecological niche as the microbial organism or organisms targeted by the bacteriocin. In some embodiments, a particular combination of and/or ratio of antimicrobial peptides is selected to target a single microbial organism (which can include targeting one or more than one microbial organisms of that type, for example clonally related microbial organisms). For example, a particular type of microbial organism may be targeted more efficiently by a predetermined mixture and/or ratio of antimicrobial peptides than by a single antimicrobial peptides.

For example, in some embodiments, an anti-fungal activity (such as anti-yeast activity) is desired for the antimicrobial peptide. A number of bacteriocins with anti-fungal activity have been identified. For example, bacteriocins from Bacillus have been shown to have neutralizing activity against yeast strains (see Adetunji and Olaoye (2013) Malaysian Journal of Microbiology 9: 130-13, hereby incorporated by reference in its entirety), an Enterococcus faecalis peptide (WLPPAGLLGRCGRWFRPWLLWLQ SGAQY KWLGNLFGLGPK, SEQ ID NO: 1) has been shown to have neutralizing activity against Candida species (see Shekh and Roy (2012) BMC Microbiology 12: 132, hereby incorporated by reference in its entirety), and bacteriocins from Pseudomonas have been shown to have neutralizing activity against fungi such as Curvularia lunata, Fusarium species, Helminthosporium species, and Biopolaris species (Shalani and Srivastava (2008) The Internet Journal of Microbiology. Volume 5 Number 2. DOI: 10.5580/27dd — accessible on the worldwide web at archive.ispub.com/journal/the-internet-journal-of-microbiology/volume-5-number-2/screening-for- antifung al- activity-of-p seudomonas-fluorescens -against-phytopathogenic-fungi.html#sthash.d0Ys03U0 .1DKuT1US .dpuf, hereby incorporated by reference in its entirety). By way of example, botrycidin AJ1316 (see Zuber, P et al. (1993) Peptide Antibiotics. In Bacillus subtilis and Other Gram-Positive Bacteria: Biochemistry, Physiology, and Molecular Genetics ed. Sonenshein et al., pp. 897-916, American Society for Microbiology, hereby incorporated by reference in its entirety) and aurin B1 (see Shenin et al. (1995) Antibiot Khimioter 50: 3-7, hereby incorporated by reference in its entirety) from B. subtilis have been shown to have antifungal activities. As such, in some embodiments, for example embodiments in which neutralization of a fungal microbial organism is desired, a bacteriocin comprises at least one of botrycidin AJ1316 or alirin B 1.

For example, in some embodiments, antimicrobial peptide activity in a particular environment, e.g., culture of a particular microbial community, is desirable, and antimicrobial peptides are selected in predetermined cocktail and/or ratios in order to kill or arrest the growth of undesired microbial organisms. In some embodiments, antimicrobial peptides are selected in predetermined cocktail and/or ratios in order to kill or arrest the growth of undesired microbial organisms different from desired microbial organism(s). Bacteriocins typically produced by the desired microorganisms can be selected, as the desired microbial organisms can already produce the relevant immunity modulators against these bacteriocins, or can readily be engineered to produce the immunity modulators. As such, the selected bacteriocins can target undesired microbial cells (including undesired microbial cells that are not yet present in the microbial population), while causing little or no neutralization of the desired microbial organisms. For example, in some embodiments, antimicrobial peptides are selected in particular ratios in order to neutralize invading microbial organisms typically found in a cyanobacteria culture environment, while preserving the cyanobacteria. Clusters of conserved bacteriocin polypeptides have been identified in a wide variety of cyanobacteria species. For example, at least 145 putative bacteriocin gene clusters have been identified in at least 43 cyanobacteria species, as reported in Wang et al. (2011), Genome Mining Demonstrates the Widespread Occurrence of Gene Clusters Encoding Bacteriocins in Cyanobacteria. PLoS ONE 6(7): e22384, hereby incorporated by reference in its entirety. Exemplary cyanobacteria bacteriocins are shown in SEQ ID NO' s 420, 422, 424, 426, 428, 430, 432, 434, 436, 438, 440, 442, 444, 446, 448, and 450.

In some embodiments, one or more antimicrobial peptide activities are selected, and a pro-polypeptide comprising the antimicrobial peptides in a desired stoichiometry is provided. The antimicrobial peptides of some embodiments can be administered or produced in a pro-polypeptide, which comprises one or more antimicrobial peptide sequences, and which can be cleaved to produce the individual antimicrobial peptides. The pro-polypeptide can comprise copy numbers of individual antimicrobial peptides such that, upon cleavage, the antimicrobial peptides are present in a particular stoichiometry. As such, a mixture of two or more different antimicrobial peptides can be administered or produced in desired ratios or stoichiometries. Methods of making antimicrobial peptides in desired ratios and desired stoichiometries are described, for example, in PCT Pub. No. WO 2019/046577, which is incorporated by reference herein in its entirety. In some embodiments, the pro-polypeptide is produced by the translational machinery (e.g. a ribosome, etc.) of a microbial cell. The pro-polypeptide can undergo cleavage (for example processing by a cleavage enzyme such as a naturally-occurring or synthetic protease) to yield the polypeptide of the antimicrobial peptide itself. As such, in some embodiments, an antimicrobial peptide is produced from a precursor polypeptide. A polynucleotide encoding the pro-polypeptide can be prepared, for example using nucleic acid synthesis and/or molecular cloning, and can be used to produce the pro-polypeptide.

In some embodiments, antimicrobial peptides (and ratios thereof) may be selected based on their ability to limit the growth of particular useful microbial strains in an environment, for example in an industrial feedstock, or in a fermenter, or in a food, pharmaceutical, or cosmetic manufacturing environment, or in a tissue environment such as a gut or skin microbiome, or in maintaining or tuning a microbial population in a plant, a plant root, and/or soil, or in preserving or maintaining the quality of a food, drug or cosmetic product. In some embodiments, antimicrobial peptides (and ratios thereof) may be selected based on their ability to neutralize one or more invading organisms which are likely to attempt to grow in a particular culture. In some embodiments, one or more antimicrobial peptide activities (and/or ratios) are selected based on one or more microbial strains or a population of microbial strains in an existing environment. For example, in some embodiments, if particular classes of invaders or likely invaders are identified in an environment, and a cocktail of neutralizing antimicrobial peptide (and ratios thereof) can be selected to neutralize the identified invaders. In some embodiments, the antimicrobial peptide are selected to neutralize all or substantially all of the microbial cells in an environment, for example to eliminate an industrial culture in a culture environment so that a new industrial culture can be introduced to the culture environment, or to prevent or inhibit contamination of a pharmaceutical or cosmetic manufacturing environment, or to prevent or minimize contamination or spoilage of a food, drug, or cosmetic product.

Suitable antimicrobial peptides include those that are susceptible to being inactivated by a protease. For example, suitable antimicrobial peptides include those that may be digested by a protease and thereby be inactivated. In some embodiments, the antimicrobial peptide administered to an environment includes one or more cleavage sites of a protease. As used herein, “cleavage site” has its customary and ordinary meaning as understood by one of skill in the art in view of this disclosure. It refers to a polypeptide sequence that mediates the cleavage of a polypeptide (for example, by hydrolysis of a peptide bond) to separate a single polypeptide into two or more discrete polypeptides. In some embodiments, a cleavage site comprises, consists of, or consists essentially of a consensus polypeptide sequence for cleavage by a protease. In some embodiments, the protease is a wild-type, a variant, or a synthetic protease, for example a wild-type, variant, or synthetic endopeptidase. By way of example, a protease can be selected for suitable protease activity in vivo or in vitro.

In some embodiments, the cleavage site comprises, consists essentially of, or consists of a cleavage site for Arg-C proteinase, Asp-N endopeptidase, BNPS-Skatole, Caspase 1, Caspase 2, Caspase 3, Caspase 4, Caspase 5, Caspase 6, Caspase 7, Caspase 8, Caspase 9, Caspase 10, Chymotrypsin-high specificity, Clostripain (Clostridiopeptidase B), CNBr, Enterokinase, Factor Xa, Formic acid, Glutamyl endopeptidase, GranzymeB, Hydroxylamine, Iodosobenzoic acid, LysC, Neutrophil elastase, NTCB (2-nitro-5-thiocyanobenzoic acid), Pepsin (pH1.3). Pepsin (pH>2), Proline-endopeptidase, Proteinase K, Staphylococcal peptidase I, Thermolysin, Thrombin, or Trypsin. As such, in the methods of some embodiments herein, one or more of the listed proteases is used to cleave the cleavage sites.

In some embodiments, the cleavage site comprises, consists essentially of, or consists of a chemical- or pH-sensitive cleavage site. Such cleavage can undergo digestion by a protease in the presence of a suitable chemical (in the case of chemical-sensitive), or at a suitable pH (in the case of pH-sensitive).

### Proteases

As used herein, “protease” or “proteinase” has their customary and ordinary meaning as would be understood by one of ordinary skill in the art in view of this disclosure. They refer to a class of enzymes that digest polypeptides containing a cleavage site specifically cleaved by the enzyme. Suitable proteases for use in embodiments of the present disclosure include those that are capable of inactivating an antimicrobial peptide administered to the environment upon cleavage of the antimicrobial peptide by the protease. In some embodiments, the protease is an enzyme that hydrolyzes peptide bonds, thus digesting a protein or polypeptide. It will be appreciated that proteases may also accomplish digestion or proteolysis that other mechanisms, for example an elimination reaction performed by a peptide lyase. Additionally, some proteases may exhibit different levels of protease activity under different conditions, for example having maximum or optimum protease activity under certain conditions. In some embodiments, a protease is selected from the group consisting of an acidic protease, an alkaline protease, or a neutral protease. Naturally-occurring proteases, variants and modifications of naturally-occurring proteases, and synthetic proteases, inter alia, are contemplated.

The protease may be a naturally occurring enzyme, a genetically engineered protease, a chemically engineered enzyme, or a synthetic protease. Suitable proteases include polypeptides derived from any source (e.g., derived from prokaryotes, or eukaryotes, such as mammals, fungi, plants, etc., or partially or fully synthetic) that digest polypeptides.

In some embodiments, the protease is (or is derived from) an acidic protease, i.e., a protease characterized by the ability to cleave proteins under acidic conditions below pH 7, e.g., at a pH between 2-7. In some embodiments the acidic protease has an optimum pH in the range from 1 and 4.

In some embodiments, the protease is (or is derived from) an alkaline protease, i.e., a protease characterized by the ability to cleave proteins under alkaline conditions above pH 7, e.g., at a pH between 7-11. In some embodiments, the alkaline protease has an optimum pH in the range from 7 and 11.

In some embodiments, the protease is (or is derived from) a neutral protease, i.e., a protease characterized by the ability to cleave proteins under conditions between pH 5 and 8. In some embodiments, the neutral protease has an optimum pH in the range between 5 and 8.

Suitable proteases include, without limitation, Arg-C proteinase, Asp-N endopeptidase, BNPS-Skatole, Caspase 1, Caspase 2, Caspase 3, Caspase 4, Caspase 5, Caspase 6, Caspase 7, Caspase 8, Caspase 9, Caspase 10, Chymotrypsin-high specificity, Clostripain (Clostridiopeptidase B), CNBr, Enterokinase, Factor Xa, Formic acid, Glutamyl endopeptidase, GranzymeB, Hydroxylamine, Iodosobenzoic acid, LysC, Neutrophil elastase, NTCB (2-nitro-5-thiocyanobenzoic acid), Pepsin (pH1.3), Pepsin (pH>2), Proline-endopeptidase, Proteinase K, Staphylococcal peptidase I, Thermolysin, Thrombin, or Trypsin.

“Serpin” as used herein has its customary and ordinary meaning as would be understood by one of ordinary skill in the art in view of this disclosure. Serpin may refer to a class of polypeptides that inhibit protease activity (e.g., protein-digesting activity of a protease). The serpin may be naturally occurring, genetically engineered, chemically engineered, or synthetic. Suitable serpins include polypeptides derived from any source (e.g., derived from prokaryotes, or eukaryotes, such as mammals, fungi, plants, etc., or partially or fully synthetic) that inhibits the activity of an antimicrobial peptide administered to the environment. In modulation methods, compositions, and kits of some embodiments, a serpin is provided to inhibit the activity of a protease, for example once the protease is no longer needed. In modulation methods, compositions, and kits of some embodiments, a serpin is administered to the environment after the antimicrobial peptide is digested by the protease.

Suitable serpins include, without limitation, al-antichymotrypsin (SERPINA3), α2-antiplasmin (SERPINF2) antithrombin (ATIII) (SERPINC1), heparin cofactor II (HCII) (SERPIND1), protein C inhibitor (PCI) (SERPINA5), α1-antitrypsin (α1AT) (SERPINA1), Kallistatin (SERPINA4), Plasminogen activator inhibitor-1 (SERPINE1), Cl-esterase inhibitor (SERPING1), protease nexin 1 (SERPINE2) or Protein Z-dependent inhibitor (SERPINA10), neuroserpin (NS), plasminogen activator inhibitor 2 (serpin B2), maspin (serpin B5), and Bikunin.

### Nucleic Acids

In accordance with the modulation methods, compositions and kits of some embodiments described herein, an antimicrobial peptide (e.g., bacteriocin) and/or protease is encoded by a nucleic acid, such as a DNA, RNA, or combination of these. For example, a DNA sequence of an antimicrobial peptide (e.g., bacteriocin) gene may encode an mRNA transcript that is translated into a protein comprising, consisting essentially of, or consisting of an antimicrobial peptide (such as a bacteriocin). In some cases, a DNA sequence of protease gene may encode an mRNA transcript that is translated into a protein comprising, consisting essentially of, or consisting of a protease. It is contemplated that a nucleic acid may comprise one or more non-naturally-occurring nucleotides, for example, locked nucleic acids (LNA), peptide nucleic acid (PNA), and the like. In the modulation methods of some embodiments herein, polynucleotides encoding pro-polypeptides can be delivered to microorganisms, and can be stably integrated into the chromosomes of these microorganisms, or can exist free of the genome, for example in a plasmid, extrachromosomal array, episome, minichromosome, or the like.

Exemplary vectors for genetic modification of microbial cells include, but are not limited to, plasmids, extrachromosomal arrays, episomes, minichromosomes, viruses (including bacteriophage), and transposable elements. Additionally, it will be appreciated that entire microbial genomes comprising desired sequences can be synthesized and assembled in a cell (see, e.g. Gibson et al. (2010), Science 329: 52-56). As such, in some embodiments, a microbial genome (or portion thereof) is synthesized with desired features such as bacteriocin polynucleotide(s) and/or protease polynucleotide(s), and introduced into a microbial cell.

In some embodiments, a cassette for inserting one or more desired bacteriocin and/or protease polynucleotides into a polynucleotide sequence (for example inserting, into an expression vector, a cassette encoding a pro-polypeptide comprising bacteriocins) is provided. Exemplary cassettes include, but are not limited to, a Cre/lox cassette or FLP/FRT cassette. In some embodiments, the cassette is positioned on a plasmid, so that a plasmid with the desired polynucleotide encoding the desired pro-polypeptide can be readily introduced to the microbial cell. In some embodiments, the cassette is positioned in a desired position in the genome of the microbial cell.

In some embodiments, plasmid conjugation can be used to introduce a desired plasmid from a “donor” microbial cell to a recipient microbial cell. Goñi-Moreno, et al. (2013) Multicellular Computing Using Conjugation for Wiring. PLoS ONE 8(6): e65986, hereby incorporated by reference in its entirety. For example, a genetically modified desired microbial organism of some embodiments can introduce a plasmid encoding an antimicrobial peptide to another microbial organism in the microbial community. In some embodiments, plasmid conjugation can genetically modify a recipient microbial cell by introducing a conjugation plasmid from a donor microbial cell to a recipient microbial cell. Without being limited by any particular theory, conjugation plasmids that comprise the same or functionally same set of replication genes typically cannot coexist in the same microbial cell. As such, in some embodiments, plasmid conjugation “reprograms” a recipient microbial cell by introducing a new conjugation plasmid to supplant another conjugation plasmid that was present in the recipient cell. In some embodiments, plasmid conjugation is used to engineer (or reengineer) a microbial cell with a particular nucleic acid encoding an antimicrobial peptide and/or protease. According to some embodiments, a variety of conjugation plasmids comprising different nucleic acids comprising a variety of different antimicrobial peptides and/or proteases is provided. The plasmids can comprise additional genetic elements as described herein, for example promoters, translational initiation sites, and the like. In some embodiments the variety of conjugation plasmids is provided in a collection of donor cells, so that a donor cell comprising the desired plasmid can be selected for plasmid conjugation.

In some embodiments, a particular combination and/or ratio of antimicrobial peptides is selected, and an appropriate donor cell (encoding the particular pro-polypeptide) is conjugated with a microbial cell of interest to introduce a conjugation plasmid comprising that combination into a recipient cell.

### Secretion Signals

To facilitate secretion of an antimicrobial peptide or protease, the antimicrobial peptide or protease may further comprise a suitable secretion signal. Secretory systems across taxa have been shown to share core features, including an integral membrane translocation apparatus, which is canonically a heterotrimeric protein complex such as the SecYEG complex in bacteria, or the Sec61 complex in eukaryotes, or archaeal homologs of members of these complexes, such as SecY/Sec61α and SecE/Sec61γ homologs in archaea (See, e.g., Pöhlschroder et al., Cell 91: 563-566 (1997)), which is incorporated by reference in its entirety herein.

Several canonical bacterial secretion systems, which may be applicable to gram positive, gram negative, or both types of bacteria have been described, and are reviewed for example, in Green et al., Microbiol Spectr. 4: doi:10.1128/microbiolspec.VMBF-0012-2015 (2016), which is hereby incorporated by reference in its entirety herein. Table 1 of Green et al., reproduced below, highlights characteristics of the bacterial Sec, Tat, T1SS, T2SS, T3SS, T4SS, T5SS, T6SS, SecA2, Sortase, Injectosome, and T7SS secretion systems. For eukaryotic microbial organisms, suitable secretory systems may also be used. For example, the secretory pathway in the yeast S. cerevisiae has been characterized in detail (See, e.g., Novick et al., Cell 25: 461-469 (1981), which is incorporated by reference in its entirety herein).

TABLE 1
Classes of bacterial secretion systems

| | | | Folded | Number | Gram (+) |
|---|---|---|---|---|---|
| Secretion | Secretion | Steps in | Sub- | of Mem- | or |
| Apparatus | Signal | Secretion | strates? | branes | Gram (−) |
| Sec | N-terminus | 1 | No | 1 | Both |
| Tat | N-terminus | 1 | Yes | 1 | Both |
| T1SS | C-terminus | 1 | No | 2 | Gram (−) |
| T2SS | N-terminus | 2 | Yes | 1 | Gram (−) |
| T3SS | N-terminus | 1-2 | No | 2-3 | Gram (−) |
| T4SS | C-terminus | 1 | No | 2-3 | Gram (−) |
| T5SS | N-terminus | 2 | No | 1 | Gram (−) |
| T6SS | No known | 1 | Un- | 2-3 | Gram (−) |
| | secretion | | known | | |
| | signal | | | | |
| SecA2 | N-terminus | 1 | No | 1 | Gram (+) |
| Sortase | N-terminus | 2 | Yes | 1 | Gram (+) |
| | (Sec) | | | | |
| | C-terminus | | | | |
| | (cws) | | | | |
| Injectosome | N-terminus | 2 | Yes | 1 | Gram (+) |
| T7SS | C-terminus | 1 | Yes | 1-3 | Gram (+) |

Translocation to the extracellular environment may be initiated by a signal sequence. Signal sequences are canonically conserved across the domains of life, and, by way of example, N-terminal signal sequences may comprise a positively charged N terminus, a core of hydrophobic amino acid residues, and a more polar C terminus (Pöhlschroder et al., Cell 91: 563-566 (1997)). Optionally, for a C-terminal signal sequence, these features may be in reverse order. Various informatics tools are available to identify predicted prokaryotic and eukaryotic signal sequences, for example, SignalP 5.0, as described in Armenteros et al., Nature Biotechnology, 37, 420-423 (2019), which is incorporated by reference in its entirety. The current version of SignalP is accessible on the world wide web at www.cbs.dtu.dk/services/SignalP.

It will be appreciated that in accordance with embodiments herein, the desired microbial organism is known, and as such, a suitable secretion signal (compatible with a relevant secretion system) may be selected.

### Immunity Modulators

In some embodiments, a particular immunity modulator or particular combination of immunity modulators confers immunity to a particular antimicrobial peptide (e.g., bacteriocins, etc.), particular class or category of antimicrobial peptides, or particular combination of antimicrobial peptides. Exemplary antimicrobial peptides (e.g., bacteriocins, etc.) to which immunity modulators can confer immunity are identified in Table 2 of U.S. Pat. No. 9,333,227. Example immunity modulator sequences include, for example, any of SEQ ID NOs: 452-540 (even) and SEQ ID NOs: 453-541 (odd).

While Table 2 of U.S. Pat. No. 9,333,227 and the present sequence listing identifies an “organism of origin” for exemplary immunity modulators, these immunity modulators can readily be expressed in other naturally-occurring, genetically modified, or synthetic microorganisms to provide a desired antimicrobial peptide immunity activity in accordance with some embodiments herein. As such, as used herein “immunity modulator” has its customary and ordinary meaning as understood by one of skill in the art in view of this disclosure, and refers not only to structures expressly provided herein, but also to structure that have substantially the same effect as the “immunity modulator” structures described herein, including fully synthetic immunity modulators, and immunity modulators that provide immunity to antimicrobial peptides (e.g., bacteriocins, etc.) that are functionally equivalent to the antimicrobial peptides disclosed herein.

Exemplary polynucleotide sequences encoding the polypeptides of SEQ ID NOs: 452-540 (even) are indicated in SEQ ID NOs: 453-541 (odd). The skilled artisan will readily understand that the genetic code is degenerate, and moreover, codon usage can vary based on the particular organism in which the gene product is being expressed, and as such, a particular polypeptide can be encoded by more than one polynucleotide. In some embodiments, a polynucleotide encoding an antimicrobial peptide immunity modulator is selected based on the codon usage of the organism expressing the antimicrobial peptide immunity modulator. In some embodiments, a polynucleotide encoding an antimicrobial peptide immunity modulator is codon optimized based on the particular organism expressing the antimicrobial peptide immunity modulator.

A vast range of functional immunity modulators can incorporate features of immunity modulators disclosed herein, thus providing for a vast degree of identity to the immunity modulators of SEQ ID NOs: 452-540 (even). In some embodiments, an immunity modulator has at least about 50% identity, for example, at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of the polypeptides of Table 2 of U.S. Pat. No. 9,333,227, or a range of identity defined by any two of the preceding values.

### Promoters

Promoters are well-known in the art. A promoter can be used to drive the transcription of one or more genes. In some embodiments, a promoter drives expression of polynucleotide encoding an antimicrobial peptide or protease as described herein. In some embodiments, a promoter drives expression of a polynucleotide encoding a pro-polypeptide comprising two or more antimicrobial peptides as described herein. In some embodiments, a promoter drives expression of an immunity modulator polynucleotide. In some embodiments, a promoter drives expression of polynucleotide encoding an antimicrobial peptide as described herein, but the microbial cell does not express immunity modulators for one or more of these antimicrobial peptides (for example, the cell can lack a promoter driving transcription of the immunity modulator, or can lack nucleic acid encoding the immunity modulator). In some embodiments, a promoter drives expression of a polynucleotide encoding a pro-polypeptide comprising two or more antimicrobial peptides in a microbial cell, but the microbial cell does not express immunity modulators for one or more of these antimicrobial peptides (for example, the cell can lack a promoter driving transcription of the immunity modulator, or can lack nucleic acid encoding the immunity modulator).

In some embodiments, a promoter drives expression of a protease, as described herein. In some embodiments, the promoter induces transcription of a nucleic acid encoding the protease upon a specified time point, e.g., a specified endpoint. In some embodiments, the promoter induces transcription of a nucleic acid encoding the protease such that the transcript is translated into a protease polypeptide upon a specified time point, e.g., a specified endpoint. In some embodiments, the promoter induces transcription of a nucleic acid encoding the protease such that the protease is secreted upon a specified time point, e.g., a specified endpoint. In some embodiments, the promoter induces transcription of a nucleic acid encoding the protease such that the transcript is translated into a protease pro-polypeptide, which is then activated upon a specific time point, e.g., endpoint.

Some promoters can drive transcription at all times (“constitutive promoters”). Some promoters can drive transcription under only select circumstances (“conditional promoters”), for example depending on the presence or absence of an environmental condition, chemical compound, gene product, stage of the cell cycle, or the like.

The skilled artisan will appreciate that depending on the desired expression activity, an appropriate promoter can be selected, and placed in cis with a nucleic acid sequence to be expressed. Exemplary promoters with exemplary activities, and useful in some embodiments herein are provided in SEQ ID NOs: 544-698 herein. The skilled artisan will appreciate that some promoters are compatible with particular transcriptional machinery (e.g. RNA polymerases, general transcription factors, and the like). As such, while compatible “species” are identified for some promoters described herein, it is contemplated that in some embodiments, these promoters can readily function in microorganisms other than the identified species, for example in species with compatible endogenous transcriptional machinery, genetically modified species comprising compatible transcriptional machinery, or fully synthetic microbial organisms comprising compatible transcriptional machinery.

The promoters of SEQ ID NOs: 544-698 herein are publicly available from the Biobricks foundation. It is noted that the Biobricks foundation encourages use of these promoters in accordance with BioBrick™ Public Agreement (BPA). The promoters of SEQ ID NOs: 544-698 are provided by way of non-limiting example only. The skilled artisan will readily recognize that many variants of the above-referenced promoters, and many other promoters (including promoters isolated from naturally existing organisms, variations thereof, and fully synthetic promoters) can readily be used in accordance with some embodiments herein.

It should be appreciated that any of the “coding” polynucleotides described herein (for example an antimicrobial peptide polynucleotide, immunity polynucleotide, or nucleotide encoding a pro-polypeptide comprising two or more antimicrobial peptides) is generally amenable to being expressed under the control of a desired promoter. In some embodiments, a single “coding” polynucleotide is under the control of a single promoter. In some embodiments, two or more “coding” polynucleotides are under the control of a single promoter, for example two, three, four, five, six, seven, eight, nine, or ten polynucleotides.

Generally, translation initiation for a particular transcript is regulated by particular sequences at or 5′ of the 5′ end of the coding sequence of a transcript. For example, a coding sequence can begin with a start codon configured to pair with an initiator tRNA. While naturally-occurring translation systems typically use Met (AUG) as a start codon, it will be readily appreciated that an initiator tRNA can be engineered to bind to any desired triplet or triplets, and accordingly, triplets other than AUG can also function as start codons in some embodiments. Additionally, sequences near the start codon can facilitate ribosomal assembly, for example a Kozak sequence ((gcc)gccRccAUGG, SEQ ID NO: 542, in which R represents “A” or “G”) or Internal Ribosome Entry Site (IRES) in typical eukaryotic translational systems, or a Shine-Delgarno sequence (GGAGGU, SEQ ID NO: 543) in typical prokaryotic translation systems. As such in some embodiments, a transcript comprising a “coding” polynucleotide sequence, for example an antimicrobial peptide polynucleotide or immunity polynucleotide, or nucleotide encoding a pro-polypeptide comprising two or more antimicrobial peptides, comprises an appropriate start codon and translational initiation sequence. In some embodiments, for example if two or more “coding” polynucleotide sequences are positioned in cis on a transcript, each polynucleotide sequence comprises an appropriate start codon and translational initiation sequence(s). In some embodiments, for example if two or more “coding” polynucleotide sequences are positioned in cis on a transcript, the two sequences are under control of a single translation initiation sequence, and either provide a single polypeptide that can function with both encoded polypeptides in cis.

### Methods of Modulating the Activity of Antimicrobial Peptides

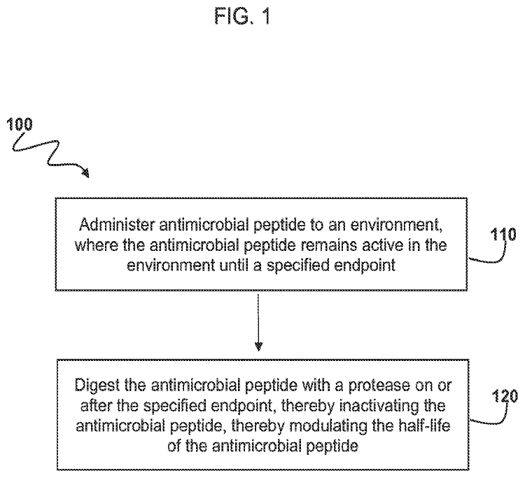

With reference to FIG. 1, a flow diagram of a method of modulating the half-life of an antimicrobial peptide (a “modulation method”) 100 according to some embodiments is shown. The modulation method may include administering 110 an antimicrobial peptide to an environment, in which the antimicrobial peptide remains active in the environment until a specified endpoint; and digesting 120 the antimicrobial peptide with a protease on or after the specified endpoint, thereby inactivating the antimicrobial peptide, and thereby modulating the half-life of the antimicrobial peptide. Digesting the antimicrobial peptide with a protease may generally reduce the half-life of the antimicrobial peptide, such that the antimicrobial activity of the antimicrobial peptide decreases at a faster rate than the rate of decrease in the absence of the protease.

In some embodiments, where the antimicrobial peptide remains active in the environment until a specified endpoint, the antimicrobial activity of the antimicrobial peptide in the environment is substantially maintained at or around a desired level (e.g., a level, or effective level, of antimicrobial activity sufficient to inhibit the growth or reproduction of an undesired microbial organism) at least until the specified endpoint. In some embodiments, where the antimicrobial peptide remains active in the environment until a specified endpoint, the antimicrobial activity of the antimicrobial peptide in the environment is substantially maintained at or above an effective level of antimicrobial activity at least until the specified endpoint. In some embodiments, the antimicrobial activity of the antimicrobial peptide in the environment is maintained at at least about 50%, at least about 60%, at least about 70%, at least about 75%, at least about 80%, at least about 85%, at least about 90%, at least about 95%, at least about 97%, at least about 100%, or a percentage defined by any two of the preceding values, (e.g., 50-60%, 60-70%, 70-75%, 75-80%, 80-85%, 85-90%, 90-95%, 95-97%, 97-100%), of the initial level of antimicrobial activity at least until the specified endpoint. In some embodiments, the antimicrobial activity of the antimicrobial peptide in the environment is not reduced by more than about 50%, about 40%, about 30%, about 25%, about 20%, about 15%, about 10%, about 5%, or about 3%, or not reduced by more than a percentage defined by any two of the preceding values (e.g., 50-40%, 40-30%, 30-25%, 25-20%, 20-15%, 15-10%, 10-5%, 5-3%), from a desired level of antimicrobial activity (e.g., a level, or effective level, of antimicrobial activity sufficient to inhibit the growth or reproduction of an undesired microbial organism), or is at the desired level, at least until the specified endpoint.

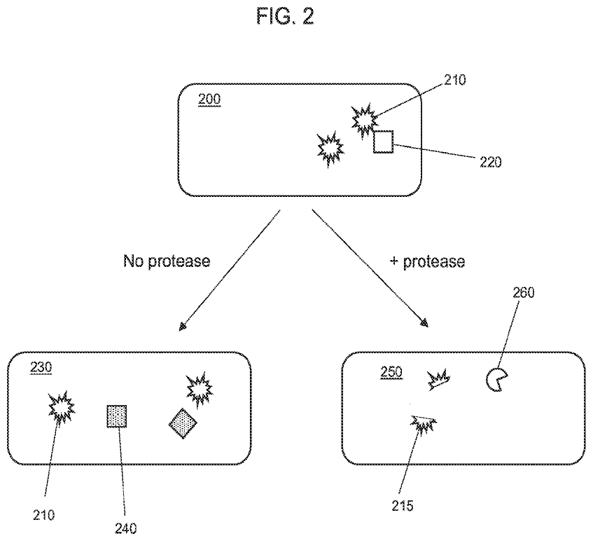

With reference to FIG. 2, a schematic diagram of a modulation method according to some non-limiting embodiments of the present disclosure is shown. The method may include administering an antimicrobial peptide 210 to an environment 200 in which the antimicrobial peptide is active (e.g., inhibits growth of a microbial organism 220) until a specified endpoint. In some embodiments, the environment is known to have, or is prone to promote the growth of, undesirable microbial organisms that are susceptible to the antimicrobial peptide. The undesirable microbial organisms may be known or unknown. In some embodiments, one or more undesirable microbial organisms are targeted by the antimicrobial peptide administered to the environment.

The method may further include digesting the antimicrobial peptide with a protease 260 on or after the specified endpoint, and the antimicrobial peptide is thereby inactivated 215. In some cases, the antimicrobial peptide may be inactivated when or after a certain period of time passes, when or after a desired decrease in the amount of a microbial organism targeted by the antimicrobial peptide is achieved, or when or after the environment changes. The environment 200 in which the antimicrobial peptide is administered and the environment 250 in which the antimicrobial peptide is inactivated may be the same environment, or different environments.

The modulation methods of some embodiments include identifying the environment as permissive for the growth of microbial organisms capable of developing resistance to the antimicrobial peptide. In some embodiments, the environment 200 is identified as being permissive for the growth of microbial organisms that can develop resistance to the antimicrobial peptide. Thus, in the continued presence of the antimicrobial peptide in the environment 230, the microbial organisms may become resistant 240 to the antimicrobial peptide. In some embodiments, the antimicrobial peptide is allowed to remain active until an endpoint after which microbial organisms targeted by the antimicrobial peptide will develop (or are expected to develop) resistance to the antimicrobial peptide. Thus, on or after such an endpoint, the antimicrobial peptide is digested to inactivate it. In some embodiments, the microbial organism targeted by the antimicrobial peptide is the same as the microbial organism that can develop resistance to the antimicrobial peptide in the environment. In some embodiments, the microbial organism targeted by the antimicrobial peptide is different from the microbial organism that may develop resistance to the antimicrobial peptide in the environment.

The environment to which the antimicrobial peptide is administered in the modulation method may be any environment in which it is desirable to modulate the half-life of the antimicrobial peptide, e.g., desirable to inhibit or prevent development of resistance to the antimicrobial peptide. Suitable environments include, without limitation, an organ of a subject (e.g., a mammalian subject, including a human subject), a microbiome in vivo, a food product or portion thereof (such as a dairy product), a medical device, an industrial feedstock, or a pharmaceutical or cosmetic manufacturing environment. Suitable organ environments include, without limitation, the gastrointestinal system, skin, lung, oral cavity, mucosa, uterus, vagina, biliary tract. Suitable microbiome includes a microbiome of a subject, or in an ecological setting. For example, the microbiome can be a microbiome selected from the group consisting of: gastrointestinal tract, skin, mammary gland, placenta, biofluid, seminal fluid, uterus, vagina, ovarian follicle, lung, saliva, oral cavity, mucosa, conjunctiva, biliary tract, and soil, or a combination of two or more of any of the listed items. A medical device may include any material that is used in the treatment, monitoring, or prophylaxis of a condition in a patient. Suitable medical devices include implantable medical devices, such as prostheses, vascular grafts, bone or cartilage implants, stents, nerve guides, heart valves, etc. Suitable medical devices include surgical tools and devices that may come in contact with a patient, such as scalpels, graspers, clamps, needle drivers, retractors, distractors, cutters, dilators, suction devices, sealing devices, needles, drills, scopes, probes, surgical tables, etc. In some embodiments, the environment is the external surface of the medical devices.

A “subject” as used herein can be any suitable subject having a microbial community in which it is desirable to modulate the half-life of the antimicrobial peptide, e.g., desirable to inhibit or prevent development of resistance to the antimicrobial peptide. A subject can be a mammal, a non-human mammal, or a non-mammalian subject. In some embodiments, a subject is, without limitation, a human, non-human primate, murine, bovine, porcine, equine, feline, canine, or avian subject. In some embodiments, a subject is a domesticated animal.

In accordance with the modulation method of some embodiments, the antimicrobial peptide may be administered to the environment using any suitable method for the particular environment. For example, in some embodiments, the antimicrobial peptide may be administered to a subject through topical, oral, parenteral, intravenous, or any other suitable routes to administer the antimicrobial peptide to an organ or in vivo microbiome environment. In such cases, the method in some embodiments may include selecting the antimicrobial peptide before administering, for example selecting the antimicrobial peptide. It is also contemplated that the antimicrobial peptide may be expressed and secreted by a microbial organism in the environment as described herein.

In accordance with the modulation method of some embodiments, the antimicrobial peptide may be administered to an industrial feedstock or manufacturing environment by any suitable methodology. In some embodiments, the antimicrobial peptide is administered to an industrial feedstock or manufacturing environment through a manifold connected to the environment. The administered amount may be any suitable amount to inhibit the growth of a microbial organism, e.g., an undesired microbial organism, in the environment, for example in an amount that is at least an LD₅₀ for the undesired microbial organism in the environment.

The modulation method according to some embodiments comprises administering the antimicrobial peptide by expressing the antimicrobial peptide in and secreting it from a genetically engineered microbial organism. For example, the environment may include a microbial organism genetically engineered with a nucleic acid encoding the antimicrobial peptide. The microbial organism may be genetically engineered with any suitable nucleic acid encoding the antimicrobial peptide as described herein. In some embodiments, the nucleic acid may be configured with suitable promoters and other regulatory elements such that the antimicrobial peptide is expressed and secreted by the microbial organism in the environment.

In a modulation method according to some embodiments, the antimicrobial peptide administered to the environment remains active until a specified endpoint. The endpoint may be any suitable endpoint, upon which it is acceptable to degrade the antimicrobial peptide. Suitable endpoint may be, without limitation, a lapse of a specified amount of time, a change in an activity level or quantity or growth rate of a microbial organism targeted by the antimicrobial peptide, or a change in the environment, or a combination of two or more of the listed items. In some embodiments, the specified endpoint is 30 minutes or more, e.g., one hour or more, 6 hours or more, 12 hours or more, 1 day or more, 3 days or more, 1 week or more, 2 weeks or more, 1 month or more, 3 months or more, 6 months or more, or 1 year or more, after administering the antimicrobial peptide to the environment. In some embodiments, the specified endpoint is between 30 minutes and 1 year, e.g., between 1 hour and 6 months, between 1 hour and 3 months, between 6 hours and 1 month, between 6 hours and 2 weeks, between 12 hours and 1 week, including between 1 day and 5 days after administering the antimicrobial peptide to the environment.

In some embodiments, it may be desirable for the antimicrobial peptide to remain active until microbial organisms targeted by the antimicrobial peptide are reduced or are eliminated from the environment. In some embodiments, the specified endpoint is a change in a quantity, growth rate, or activity level of a microbial organism (e.g., undesirable microbial organism, such as a contaminant or a pathogen) targeted by the antimicrobial peptide past a threshold level. The quantity, growth rate, or activity level may be any suitable measure of quantity, growth rate, or activity of the target microbial organism, including, without limitation, the population, population density, growth rate, amount of toxin or harmful substances produced by the microbial organism, in the environment, or a combination of two or more of the listed items. In some embodiments, the specified endpoint is a reduction in the quantity, growth rate, or activity level below a threshold. In some embodiments, the specified endpoint is a reduction in the quantity, growth rate, or activity level below a predetermined reference level of activity. In some embodiments, the specified endpoint is a reduction in the quantity, growth rate, or activity level relative to a pre-administration level of activity. In some embodiments, the specified endpoint is a reduction in the quantity, growth rate, or activity level of 10% or more, e.g., 20% or more, 30% or more, 40% or more, 50% or more, 60% or more, 70% or more, 80% or more, 90% or more, 95% or more, including about 100% compared to a pre-administration level of activity of the target microbial organism. In the modulation method of some embodiments, the specified endpoint is a change in the environment.

In the modulation method of some embodiments, the antimicrobial peptide is digested before it leaves the environment. It is contemplated digesting the antimicrobial peptide before it leaves the environment can inhibit or prevent the development of resistance to the antimicrobial peptide outside of the environment (for example, in a second, different environment outside of the environment). Thus, in some embodiments, an antimicrobial peptide is prevented from being present in nature. In some embodiments, the antimicrobial peptide is digested no later than, or prior to, the antimicrobial peptide leaving the environment (e.g., to enter another environment that is different from the first environment). In some embodiments, the antimicrobial peptide is digested no later than, or prior to, the antimicrobial peptide leaving the environment and coming in contact with a second environment that comprises one or more microbial organisms. In some embodiments, the environment is an industrial manufacturing environment, e.g., for a food product, pharmaceutical product, cosmetic product, etc., where the specified time point is when, or before, the manufactured product leaves the manufacturing environment

In the modulation method of some embodiments, on or after the specified endpoint, the antimicrobial peptide may be digested with a protease to inactivate the antimicrobial peptide. In some embodiments, the modulation method comprises digesting the antimicrobial peptide administered to the environment with at least 1, 2, 3, 4, 5, 6, 7, 8, 9, or 10 different proteases to inactivate the antimicrobial peptide, including ranges between any two of the listed values, for example 1-5, 1-10, 2-5, 2-10, or 5-10 different proteases. The digesting may be done in any suitable manner. The modulation methods of some embodiments includes digesting the antimicrobial peptide in the environment to which the antimicrobial peptide is administered. In some embodiments, the modulation method includes administering a protease to the environment. In some embodiments, the modulation method includes digesting the antimicrobial peptide outside of the environment to which the antimicrobial peptide is administered. The digesting may be performed under any condition (such as temperature and pH) sufficient for the protease to digest and inactivate the antimicrobial peptide. In some embodiments, the environment to which the antimicrobial peptide is administered is used to manufacture a product, e.g., an industrial, pharmaceutical, cosmetic, food product or good product, and the modulation method includes digesting the antimicrobial peptide in the environment, before the product leaves the environment. In such cases, the product does not include the antimicrobial peptide when the product is outside of the environment.

The protease may be any suitable protease for digesting and inactivating the antimicrobial peptide. In some embodiments, the protease is one that cleaves a target polypeptide at a cleavage site that is present in the antimicrobial peptide.

The antimicrobial peptide may be brought to contact with the protease in any suitable manner. In some embodiments, the protease is administered to the environment. For example, the protease may added directly or indirectly to the environment, or may be expressed by a microbial organism in the environment. For example, a microbial organism in the environment may comprise a nucleic acid encoding the protease under the control of a promoter that initiates or increases expression of the protease at or after the specified endpoint. In some embodiments, the protease that cleaves the antimicrobial peptide is present as a pro-polypeptide (in an inactive state) before the endpoint, and may be activated on or after the endpoint. In some embodiments, the protease is disposed on a substrate, and the antimicrobial peptide is brought in contact with the substrate. In some embodiments, the substrate is a solid support, e.g., a bead, capillary, matrix, plate, membrane, well, etc. The protease may be disposed on the substrate by any suitable means. In some embodiments, the protease is attached to (covalently or non-covalently) or associated with the substrate.

In some embodiments, the activity of the protease may be regulated. For example, the modulation methods of some embodiments includes adding a serpin to the environment, where the serpin inhibits the protease. In some embodiments, the serpin is added to the environment before the antimicrobial peptide is digested by any protease. In some embodiments, the serpin is added after the protease digests the antimicrobial peptide. In some embodiments, the serpin is added in sufficient amounts to inactivate substantially all the protease.

In some embodiments, it may be desirable to administer two or more antimicrobial peptides to the environment, for example at least 2, 3, 4, 5, 6, 7, 8, 9, 10, 15, 20, 25, or 30 antimicrobial peptides, including ranges between any two of the listed values, such as 2-5, 2-10, 2-30, 5-10, 5-30, or 10-30 antimicrobial peptides. Thus, the modulation methods of some embodiments includes administering a cocktail of two or more different antimicrobial peptides to the environment. In such cases, a given protease may or may not digest one or more of the antimicrobial peptides. For example, in some embodiments, the protease digests one of two antimicrobial peptides in the cocktail of antimicrobial peptides administered to the environment, and does not digest the second antimicrobial peptide. For example, in some embodiments, the protease digests different antimicrobial peptides in the environment at different rates. In some embodiments, three different antimicrobial peptides are administered to the environment. In some embodiments, where two or more different antimicrobial peptides are administered to the environment, some or all of the different antimicrobial peptides may be digested by two or more different proteases. For example, in some embodiments, the method includes administering a cocktail of three different antimicrobial peptides that includes an antimicrobial peptide that is then digested by a protease, another antimicrobial peptide in the cocktail that is different from the first antimicrobial peptide is not digested by the protease, and a third antimicrobial peptide in the cocktail that is different from the first antimicrobial peptide is digested by a second protease that is different from the first protease.

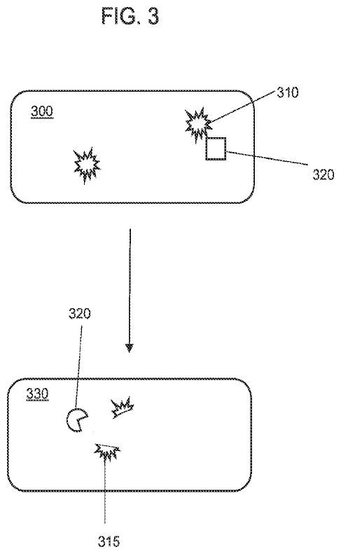

With reference to FIG. 3, a schematic diagram of a modulation method of some non-limiting embodiments is shown. The modulation methods of some embodiments may be useful to inhibit or prevent spoilage of a food product using an antimicrobial peptide. For example, the method may include selecting an antimicrobial peptide 310 that is trypsin-sensitive (e.g., the antimicrobial peptide is inactivated by digestion with trypsin). In such cases, the trypsin may be administered to an environment 300 that includes a food product, or a portion thereof, and the antimicrobial peptide inhibits the growth or reproduction of microbial organisms 320 (e.g., contaminating microbial organisms) that cause food spoilage. Upon consumption of the food product by a subject (e.g., a human subject), the specified endpoint is reached, and trypsin 340 in the digestive tract 330 of the subject may inactivate the antimicrobial peptide 315.

### Compositions

Also provided are compositions that include a protease, an antimicrobial peptide that includes a cleavage site that is specifically cleaved by the protease, and a microbial organism capable of developing resistance to the antimicrobial peptide. In some embodiments, the composition is in an environment to which the antimicrobial peptide was administered, as described herein. The protease may be any suitable protease, as described herein, for digesting and inactivating the antimicrobial peptide. The amount of protease in the composition may depend on the amount of antimicrobial peptide in the composition. In some embodiments, the amount of protease in the composition is sufficient to reduce the half-life of the antimicrobial peptide by 10% or more, e.g., by 20% or more, by 30% or more, by 40% or more, by 50% or more, by 60% or more, by 70% or more, by 80% or more, by 90% or more, by 95% or more, or about by 100% compared to the half-life of the antimicrobial peptide in the absence of the protease. In some embodiments, the composition includes a pro-polypeptide of the protease that, when activated by cleavage of the pro-domain, digests and inactivates the antimicrobial peptide. In some embodiments, the composition includes one, two, three, four, five or more proteases, as described herein, some or all of which cleaves an antimicrobial peptide in the composition. In some embodiments, the composition includes a serpin that inhibits the protease. The composition may include any suitable serpin that can inhibit the activity of the protease, as described herein.

The antimicrobial peptide may be any suitable antimicrobial peptide that includes a cleavage site that is cleaved by the protease, as described herein. The amount of antimicrobial peptide in the composition may be any suitable amount to inhibit the growth of a target microbial organism (e.g., an undesired microbial organism) in the environment. In some embodiments, the composition includes one, two, three, four, five or more different antimicrobial peptides, as described herein, some or all of which may be a cleavage target of a protease in the composition.

The microbial organism capable of developing resistance to the antimicrobial peptide may be any such microbial organism that is targeted by the antimicrobial peptide. In some embodiments, the microbial organism is, or is prone to become, an undesired microbial organism, e.g., a pathogen, contaminating microbe, or a microbe used in industrial production but has lost its industrial characteristic. The composition may include the microbial organism at any suitable amount. In some embodiments, the composition includes at least 10⁴, 10⁵, 10⁶, 10⁷, 10⁸, 10⁹, 10¹⁰, 10¹¹, or 10¹² colony forming units (cfu) of the microbial organism. In some embodiments, the composition includes at least 10², 10³, 10⁴, 10⁵, 10⁶, 10⁷, 10⁸, or 10 9 cfu of the microbial organism per milliliter of the composition, where the composition includes a liquid suspension.

In some embodiments of a composition of the present disclosure, the composition includes a product, such as a food product, pharmaceutical or medical product or a cosmetic product, as described herein.

### Kits

Also described are kits that comprise an antimicrobial peptide and a protease capable of digesting the antimicrobial peptide, in which the antimicrobial peptide targets a microbial organism capable of developing resistance to the antimicrobial peptide. The antimicrobial peptide may be any suitable antimicrobial peptide that includes a cleavage site that is cleaved by the protease, as described herein. In some embodiments, the antimicrobial peptide comprises a bacteriocin, an antibacterial peptide, an antiviral peptide, an anti-HIV peptide, an antifungal peptide, an antiparasitic peptide, or an anticancer peptide, or a combination of two or more of the listed items. The antimicrobial peptide may be selected to target any suitable microbial organism, for example an undesired microbial organism. It is contemplated that the targeted microbial organism may be capable of developing resistance to the antimicrobial peptide. The kit may comprise at least one, two, three, four, or five antimicrobial peptides, including ranges between any two of the listed values, for example 1-5 antimicrobial peptides. In some embodiments, the kit comprises a cocktail of antimicrobial peptides where the protease cannot digest at least one of the antimicrobial peptides.

The protease may be any suitable protease, as described herein, for digesting and inactivating the antimicrobial peptide. In some embodiments, the kit comprises a pro-polypeptide of the protease that, when activated by cleavage of a pro-domain, digests and inactivates the antimicrobial peptide. The kit may include at least one, two, three, four, or five proteases, as described herein, including ranges between any two of the listed values. In some embodiments, the kit comprises a serpin that inhibits the protease, as described herein. In some embodiments, the serpin is disposed with the protease and is configured to antagonize the protease.

In some embodiments, the kit comprises one or more compositions, wherein the one or more compositions comprise the antimicrobial peptide and the protease capable of digesting the antimicrobial peptide, as disclosed herein. The different components of the kit may be in the same or separate compositions in the kits of the present disclosure. In some embodiments, the antimicrobial peptide and the protease are in separate compositions. For example, the antimicrobial peptide may be in one container (e.g., vial, bottle, etc.) and the protease may be in a second container (e.g., vial, bottle, etc.) that is different from the first container containing the antimicrobial peptide. In some embodiments, the protease is physically separated from the antimicrobial peptide. For example, in some embodiments, the protease is in a physically separate compartment from the compartment containing the antimicrobial peptide.

In some embodiments, the antimicrobial peptide and the protease are in the same composition. In such cases, the protease may be disposed to not digest the antimicrobial peptide before a specified endpoint. For example, in some embodiments, the protease may be disposed with a serpin in the same composition. For example, in some embodiments, the protease may be sequestered from the antimicrobial peptide. The specified endpoint may be any suitable endpoint, as described herein.

In some embodiments, a kit is suitable for use in an in vivo environment, e.g., an organ or an in vivo microbiome of a subject. Thus, in some embodiments, one or both of the antimicrobial peptide and protease may be formulated for administration to a human subject.

### EXAMPLES

### Example 1: Modulating the Half-Life of an Antimicrobial Peptide that Inhibits or Prevents Spoilage of a Food Product

A bacteriocin active against Bacillus cereus, which can cause food poisoning, is provided to a dairy food product, to prevent contamination with B. cereus. The bacteriocin is trypsin-sensitive. When a human subject ingests the dairy food product, the bacteriocin is digested and inactivated by trypsin in the subject's digestive system, thereby modulating the half-life of the bacteriocin.

### Example 2: Modulating the Half-Life of an Antimicrobial Peptide that Inhibits Antibiotic-Resistant Strains of Enterococci

A bacteriocin that inhibits growth of vancomycin-resistant Enterococcus faecalis is applied to the surface of a medical device in preparation for a medical procedure. The bacteriocin inhibits the growth of these vancomycin-resistant enterococci. Shortly before the medical procedure, the proteinase K is applied to the surface of the medical device to digest and inactivate the bacteriocin. Optionally, a serpin that inhibits proteinase K is applied to the surface of the medical device to inhibit the proteinase K.

In at least some of the embodiments described herein, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims. For each method described herein, relevant compositions for use in the method are expressly contemplated, uses of compositions in the method, and, as applicable, methods of making a medicament for use in the method are also expressly contemplated. For example, for methods of modulating a half-life of an antimicrobial peptide, such as in an environment in vivo, an antimicrobial peptide and/or protease (and/or a nucleic acid encoding the antimicrobial peptide and/or protease) for use in the corresponding method are also contemplated. Methods of making a medicament comprising the antimicrobial peptide (and/or a nucleic acid encoding the antimicrobial peptide) for use in inhibiting or preventing antibiotic resistance are also contemplated.

What is claimed is:

1. A method of modulating a half-life of an antimicrobial peptide, the method comprising:
administering an antimicrobial peptide to an environment, wherein the antimicrobial peptide remains active in the environment until a specified endpoint;
on or after the specified endpoint, digesting the antimicrobial peptide with a protease, thereby inactivating the antimicrobial peptide, thereby modulating the half-life of the antimicrobial peptide.

2. The method of claim 1, further comprising identifying the environment as permissive for the growth of one or more microbial organisms capable of developing resistance to the antimicrobial peptide,
wherein the antimicrobial peptide is digested with the protease before any microbial organisms develop resistance to the anti-microbial peptide.

3. The method of any one of claims 1-2, wherein the antimicrobial peptide is digested within the environment.

4. The method of any one of claims 1-3, wherein the antimicrobial peptide inhibits the growth or reproduction of an undesired microbial organism.

5. The method of claim 4, wherein the antimicrobial peptide is digested with the protease before the undesired microbial organism or a clone thereof develops resistance to the antimicrobial peptide.

6. The method of any one of claims 4-5, wherein the undesired microbial organism is selected from the group consisting of a pathogen, a contaminant, and an industrial microbe that has lost an industrial characteristic.

7. The method of any one of claims 1-6, further comprising adding a serpin to the environment, wherein the serpin inhibits the protease.

8. The method of any one of claims 1-7, wherein the antimicrobial peptide is digested by the protease in the environment.

9. The method of any one of claims 1-7, wherein the antimicrobial peptide is digested by the protease outside of the environment.

10. The method of any one of claims 1-9, wherein the protease is disposed on a substrate, wherein digesting the antimicrobial peptide comprises contacting the antimicrobial peptide with the substrate.

11. The method of any one of claims 1-10, wherein the environment comprises an organ of a subject, a microbiome in vivo, a food product or portion thereof, a medical device, an industrial feedstock, or a pharmaceutical or cosmetic manufacturing environment.

12. The method of claim 11, wherein the environment comprises the organ of the subject or the microbiome in vivo, and wherein the method further comprises an ex vivo selection for the antimicrobial peptide prior to said administering.

13. The method of any one of claims 1-12, wherein the antimicrobial peptide is administered to a subject, wherein the environment comprises a pathogenic microbial organism, and wherein the specified endpoint comprises inhibiting the growth or reproduction of the pathogenic microbial organism.

14. The method of any one of claims 1-13, wherein the specified endpoint is a specified period of time, a decrease in a quantity of a microbial organism targeted by the antimicrobial peptide to a level below a threshold, a decrease in an activity level of a microbial organism targeted by the antimicrobial peptide to a level below a threshold, or a change in the environment.

15. The method of any one of claims 1-14, wherein the environment comprises a first genetically engineered microbial organism comprising a nucleic acid encoding the antimicrobial peptide, and wherein administering the antimicrobial peptide comprises the genetically engineered microbial organism expressing and secreting the antimicrobial peptide into the environment.

16. The method of claim 15 wherein the first genetically engineered microbial organism comprises a nucleic acid encoding the protease, and is configured to induce transcription of the nucleic acid encoding the protease, translation of a transcript of the nucleic acid encoding the protease, or secretion of the encoded protease upon the specified endpoint.

17. The method of any one of claims 1-16, wherein the environment comprises a second genetically engineered microbial organism comprising a nucleic acid encoding the protease, and configured to induce transcription of the nucleic acid encoding the protease, translation of a transcript of the nucleic acid encoding the protease, or secretion of the encoded protease upon the specified endpoint.

18. The method of any one of claims 1-17, wherein the antimicrobial peptide is selected from the group consisting of a bacteriocin, an antibacterial peptide, an antiviral peptide, an anti-HIV peptide, an antifungal peptide, an antiparasitic peptide, and an anticancer peptide.

19. The method of any one of claims 1-18, wherein the antimicrobial peptide is administered in a cocktail further comprising a first additional antimicrobial peptides different from the antimicrobial peptide, wherein the first additional antimicrobial peptide is not digested.

20. The method of claim 19, wherein the cocktail further comprises a second additional antimicrobial peptide that is different from the antimicrobial peptide, wherein the second additional antimicrobial peptide is digested by a second protease that is different from the protease.

21. The method of any one of claims 19-20, wherein at least one of the additional antimicrobial peptides is digested by the protease.

22. The method of any one of claims 1-21, the method further comprising selecting the antimicrobial peptide to be trypsin-sensitive,
wherein prior to the specified endpoint, the antimicrobial peptide inhibits the growth or reproduction of microbial organisms that cause food spoilage
wherein the specified endpoint is consumption of the food product, and
wherein the protease comprises trypsin.

23. The method of any one of claims 1-22, wherein the digesting decreases the half-life of the antimicrobial peptide by 50% or more.

24. The method of any one of claims 1-23, wherein said digesting the antimicrobial peptide comprises digesting the antimicrobial peptide with two or more different proteases.

25. The method of any one of claims 1-24, wherein the protease comprises a genetically engineered or synthetic protease.

26. The method of any one of claims 1-25, further comprising producing a product from the environment, wherein the antimicrobial peptide is digested prior to producing the product,
whereby the product does not comprise the antimicrobial peptide.

27. The method of claim 26, wherein the product is selected from the group consisting of an industrial product, a pharmaceutical product, a cosmetic product, and a good product.

28. A composition comprising:
a protease;
an antimicrobial peptide comprising a cleavage site that is specifically digested by the protease; and
a microbial organism capable of developing resistance to the antimicrobial peptide.

29. A kit comprising:
an antimicrobial peptide selected to target a microbial organism capable of developing resistance to the antimicrobial peptide; and
a protease capable of digesting the antimicrobial peptide.

30. The kit of claim 29, wherein the antimicrobial peptide and the protease are in separate compositions.

31. The kit of claim 29, wherein the antimicrobial peptide and the protease are in the same composition, and wherein the protease is disposed to not digest the antimicrobial peptide prior to a specified endpoint.

32. The kit of claim 31, wherein the protease is disposed with a serpin configured to antagonize the protease.

33. The kit of any one of claims 29-30, wherein the protease is physically separated from the antimicrobial peptide.

34. The kit of any one of claims 29-33, wherein the antimicrobial peptide and/or protease are formulated for in vivo administration to a human subject.

35. The kit of any one of claims 29-34, wherein the antimicrobial peptide is selected from the group consisting of a bacteriocin, an antibacterial peptide, an antiviral peptide, an anti-HIV peptide, an antifungal peptide, an antiparasitic peptide, and an anticancer peptide.

36. The kit of any one of claims 29-35, further comprising a cocktail of antimicrobial peptides comprising the antimicrobial peptide, wherein the protease is incapable of digesting at least some antimicrobial peptides of the cocktail.

37. The kit of any one of claims 29-36, further comprising a serpin selected to inhibit the protease.
